(19) United States
(12) Patent Application Publication
(10) Pub. No.: US 2014/0286903 A1
(43) Pub. Date: Sep. 25, 2014

(54) Substituted Purine Nucleosides, Phosphoramidate and Phosphordiamidate Derivatives for Treatment if Viral Infections

(71) Applicants: INHIBITEX, INC., Alpharetta, GA (US); UNIVERSITY COLLEGE CARDIFF CONSULTANTS LIMITED, Cardiff (GB)

(72) Inventors: Stanley Chamberlain, Alpharetta, GA (US); John Vernachio, Alpharetta, GA (US); Srinivas K. Battina, Cary, NC (US); Changalvala V.S. Ramamurty, Cary, NC (US); C. Srinivas Rao, Cary, NC (US); Chris McGuigan, Cardiff (GB); Andrea Brancale, Cardiff (GB)

(73) Assignees: INHIBITEX, INC., Alpharetta, GA (US); UNIVERSITY COLLEGE CARDIFF CONSULTANTS LIMITED, Cardiff (GB)

(21) Appl. No.: 14/357,358

(22) PCT Filed: Nov. 8, 2012
(86) PCT No.: PCT/US2012/064112

§ 371 Date: May 9, 2014

Related U.S. Application Data
(60) Provisional application No. 61/578,541, filed on Dec. 21, 2011.
(60) Provisional application No. 61/558,247, filed on Nov. 10, 2011.

(51) Int. Cl. C07H 19/16; A61K 31/708; A61K 31/7072; A61K 45/06; C07H 19/10; A61K 31/7076; C07H 19/20

(52) U.S. Cl. CPC C07H 19/16; A61K 31/7076; A61K 31/708; C07H 19/20; A61K 45/06; C07H 19/10; A61K 31/7072
USPC 424/85.7; 536/27.21; 514/45; 536/27.81; 536/27.61; 536/26.7; 514/48; 424/85.4; 514/44 R; 536/26.8; 514/51

(57) ABSTRACT

This invention is directed to compounds of Formula (I) having the structure that are useful in the treatment of viral infections in mammals, particularly in humans, mediated, at least in part, by a virus in the Flaviviridae family of viruses.

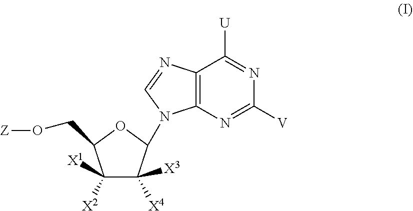

### CROSS REFERENCE TO RELATED APPLICATIONS

The present application claims the benefit of U.S. Provisional Application Ser. No. 61/558,247, filed Nov. 10, 2011, and U.S. Provisional Application Ser. No. 61/578,541, filed Dec. 21, 2011, the entire disclosure of said applications being incorporated herein by reference.

### FIELD OF THE INVENTION

This application relates to novel nucleosides, and phosphoramidates and phosphordiamidates of novel nucleosides, and their use as agents for treating viral diseases. Such compounds are inhibitors of RNA-dependant RNA viral replication and specifically, inhibitors of HCV NS5B polymerase. As inhibitors of HCV replication, such compounds are useful for treatment of hepatitis C infection in mammals.

### BACKGROUND OF THE INVENTION

HCV is a member of the Flaviviridae family of RNA viruses that affect animals and humans. The genome is a single 9.6-kilobase strand of RNA, and consists of one open reading frame that encodes for a polyprotein of approximately 3000 amino acids flanked by untranslated regions at both 5′ and 3′ ends (5′- and 3′-UTR). The polyprotein serves as the precursor to at least 10 separate viral proteins critical for replication and assembly of progeny viral particles.

Hepatitis C Virus (HCV) infection is a major health problem that leads to chronic liver disease, such as cirrhosis and hepatocellular carcinoma, in a substantial number of infected individuals, estimated to be 2-15% of the world population. There are an estimated 4.5 million infected people in the United States alone, according to the U.S. Center for Disease control. According to the World Health Organization, there are more than 200 million infected individuals worldwide, with at least 3 to 4 million people being infected each year. Once infected, about 20% of people clear the virus, but the remainder can harbor HCV for the rest of their lives.

Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring

At present, the standard treatment for chronic HCV is interferon alpha (IFN-alpha) in combination with ribavirin, which requires at least six (6) months of treatment. However, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction.

Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha and ribavirin. By now, standard therapy of chronic hepatitis C has been changed to the combination of pegylated IFN-alpha plus ribavirin. However, a number of patients still have significant side effects, primarily related to ribavirin.

Ribavirin causes significant hemolysis in 10-20% of patients treated at currently recommended doses, and the drug is both teratogenic and embryotoxic. Even with recent improvements, a substantial fraction of patients do not respond with a sustained reduction in viral load and there is a clear need for more effective antiviral therapy of HCV infection.

A number of other approaches are being pursued to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4A protease/helicase and the NS5b RNA-dependent RNA polymerase are considered the most promising viral targets for new drugs.

A number of patents disclose and claim inventions relating to CV NS5B inhibitors. For example, WO 2006/046039, WO 2006/046030 and WO 2006/029912, incorporated by reference herein, relate to tetracyclic indole compounds and pharmaceutically acceptable salts thereof, for the treatment or prevention of infection by hepatitis C virus. WO 2005/080399, incorporated by reference herein, relates to fused heterotetracyclic compounds, pharmaceutically acceptable salts thereof; and their use in aiding to remedy hepatitis C infection as potent (HCV) polymerase inhibitors. WO 2003007945, incorporated by reference herein, relates to HCV NS5B inhibitors. Further, WO 2003010140, incorporated by reference herein, relates to specific inhibitors of RNA dependent RNA polymerases, particularly viral polymerases within the Flaviviridae family, more particularly to HCV polymerase. WO 200204425, incorporated by reference herein, relates to specific inhibitors of RNA dependent RNA polymerases, particularly viral polymerases within the Flaviviridae family, and more particularly the NS5B polymerase of HCV. WO 200147883, incorporated by reference herein, relates to specific fused-ring compounds or the like or pharmaceutically acceptable salts thereof. Such compounds and salts exhibit an anti-HCV (hepatitis C virus) activity by virtue of their inhibitory activity against HCV polymerase, thus being useful as therapeutic or preventive agents for hepatitis C.

However, in view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

This invention is directed to novel compounds that are useful in the treatment of viral infections in mammals, particularly in humans, mediated at least in part, by a virus in the Flaviviridae family of viruses. According to some embodiments, the present invention provides for novel compounds of Formula I having the structure:

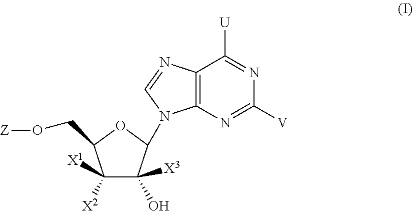

wherein
U and V are each independently selected from the group consisting of
   hydrogen
   OH
   Cl
   Br
   I
   OR1
   NH2
   NHR2
   NR2R3
   SH
      and
   SR4;
wherein
   R1, R2, R3, and R4 are independently C1-C6 alkyl or aryl(C1-C3)alkyl;
or
R2 and R3, together with the nitrogen atom to which they are attached, may join to form a 4-6 membered ring;
X1 is H or F;
X2 is F or H, with the requirement that X1≠X2;
X3 is CH3 or C1-C6 alkyl;
Z is selected from the group consisting of
   hydrogen
   —P(O)(OAr)NHR5
   —P(O)(NHR5)2
   —P(O)(NHR5)(NHR6)
   —P(O)(OH)NHR6
   —P(O)(OH)2 (monophosphate)
      and
   —P(O)(OH)—O—P(O)(OH)—O—P(O)(OH)2 (triphosphate);
wherein
   R5 and R6 are independently
      —C(R7)(R8)C(O)OR9;
      wherein
         R7 and R8 are independently
         hydrogen,
         alkyl,
         aryl(C1-C6)alkyl,
         or
         phenyl;
         R9 is independently C1-C6 alkyl,
         aryl(C1-6)alkyl,
         or
         (4-pyranyl);
   and
Ar is independently selected from the group consisting of
   phenyl
   1-naphthyl
   2-naphthyl

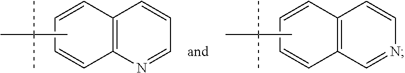

and the tautormers and the pharmaceutically acceptable salts thereof.

The anomeric linkage between the sugar moiety and the aglycone of Formula I may be of either β-D or α-D configuration.

According to some embodiments, the present invention also provides for novel compounds of Formula II or III having the structures:

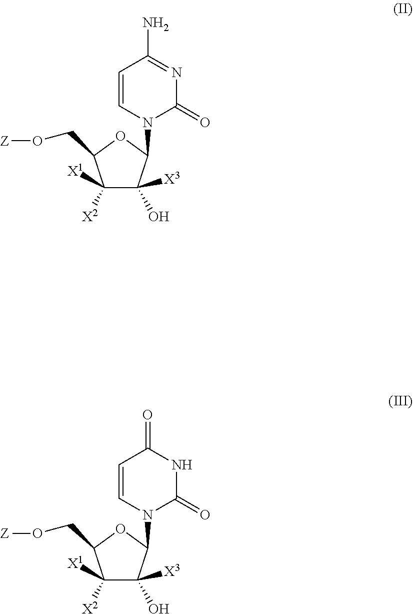

X1 is H or F;
X2 is F or H, with the requirement that X1≠X2;
X3 is CH3 or C1-C6 alkyl;
Z is selected from the group consisting of
   hydrogen
   —P(O)(OAr)NHR5
   —P(O)(NHR5)2
   —P(O)(NHR5)(NHR6)
   —P(O)(OH)NHR6
   —P(O)(OH)2 (monophosphate)
      and
   —P(O)(OH)—O—P(O)(OH)—O—P(O)(OH)2 (triphosphate);
wherein
   R5 and R6 are independently
      —C(R7)(R8)C(O)OR9;
      wherein
         R7 and R8 are independently
         hydrogen,
         alkyl,
         aryl(C1-C6)alkyl,
         or
         phenyl;
         R9 is independently C1-C6 alkyl,
         aryl(C1-C6)alkyl,
         or
         (4-pyranyl);
   and
Ar is independently selected from the group consisting of
   phenyl
   1-naphthyl
   2-naphthyl

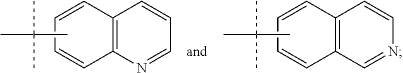

and the tautormers and the pharmaceutically acceptable salts thereof.

According to other embodiments, the present invention extends to a pharmaceutical composition comprising one or more compounds of Formula I and a pharmaceutically acceptable carrier, excipient or diluent. The pharmaceutically acceptable carrier, excipient or diluent may be pure sterile water, phosphate buffered saline or an aqueous glucose, solution.

Also provided are methods for treating a viral infection in a mammal, particularly in humans, mediated at least in part by a virus in the Flaviviridae family wherein an instant method comprises administering to a mammal that has been diagnosed with said viral infection an effective amount of a pharmaceutical composition comprising compounds of Formula I, Formula II or Formula III.

Also provided are methods for treating a viral infection in a human or animal patient that is mediated at least in part by a virus in the Flaviviridae family wherein an instant method comprises administering to a human or animal patient in need thereof an effective amount of a pharmaceutical composition comprising compounds of Formulas I, II or III.

In one embodiment, the virus is hepatitis C virus (or HCV). The present methods further extend to combination treatment comprising administration of a therapeutically effective amount of one or more agents active against hepatitis C virus. Such active agents against hepatitis C virus may include interferon-alpha or pegylated interferon-alpha alone or in combination with ribavirin or levovirin.

### DETAILED DESCRIPTION OF THE INVENTION

The present invention relates to chemical compounds, their preparation and their use in the treatment of viral infections in mammals, particularly in humans. Particularly, although not exclusively, the present invention relates to chemical compounds useful as anti-hepatitis C virus (HCV) agents.

Specifically, the present invention describes certain nucleoside diphosphoramidates, their synthesis, and their use as precursors to inhibitors of RNA-dependent RNA viral polymerase, particularly their use as precursors to inhibitors of hepatitis C virus (HCV) NS5-B polymerase, as precursors to inhibitors of HCV replication, and for the treatment of hepatitis C infection.

It is an object of the present invention to provide novel chemical compounds useful for treatment of viral infections in mammals, specifically for treatment of hepatitis C infection in mammals and particularly in humans.

The present invention relates to novel compounds of Formula I having the structure: present invention provides for novel compounds of Formula I having the structure:

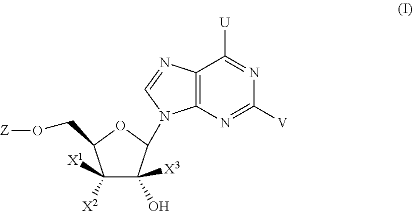

wherein
U and V are each independently selected from the group consisting of
   hydrogen
   OH
   Cl
   Br
   I
   OR1
   NH2
   NHR2
   NR2R3
   SH
      and
   SR4;
wherein
R1, R2, R3, and R4 are independently C1-C6 alkyl or aryl(C1-C3)alkyl;
X1 is H or F;
X2 is F or H, with the requirement that X1≠X2;
X3 is CH3 or C1-C6 alkyl;
Z is selected from the group consisting of
   hydrogen
   —P(O)(OAr)NHR5
   —P(O)(NHR5)2
   —P(O)(NHR5)(NHR6)
   —P(O)(OH)NHR6
   —P(O)(OH)2 (monophosphate)
      and
   —P(O)(OH)—O—P(O)(OH)—O—P(O)(OH)2 (triphosphate);
wherein
   R5 and R6 are independently
      —C(R7)(R8)C(O)OR9;
      wherein
         R7 and R8 are independently
         hydrogen,
         alkyl,
         aryl(C1-C6)alkyl,
         or
         phenyl;
         R9 is independently C1-C6 alkyl or aryl(C1-C6)alkyl;
   and
Ar is independently selected from the group consisting of
   phenyl
   1-naphthyl
   2-naphthyl

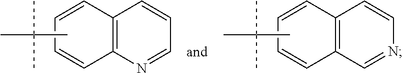

and
the tautomers and the pharmaceutically acceptable salts thereof.

The anomeric linkage between the sugar moiety and the aglycone of Formula I may be of either β-D or α-D configuration.

In addition, the present invention also provides for novel compounds of Formula II or III having the structures:

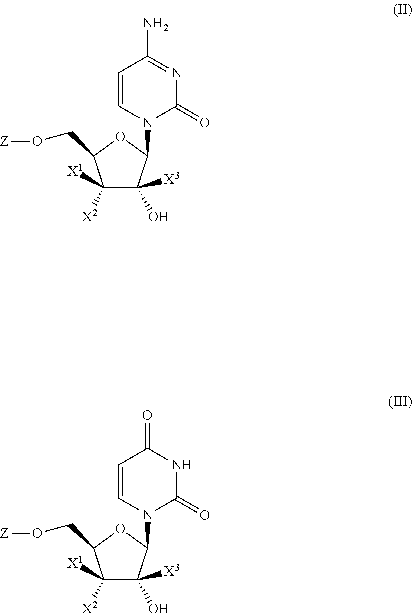

X1 is H or F;
X2 is F or H, with the requirement that X1≠X2;
X3 is CH3 or C1-C6 alkyl;
Z is selected from the group consisting of
   hydrogen
   —P(O)(OAr)NHR5
   —P(O)(NHR5)2
   —P(O)(NHR5)(NHR6)
   —P(O)(OH)NHR6
   —P(O)(OH)2 (monophosphate)
      and
   —P(O)(OH)—O—P(O)(OH)—O—P(O)(OH)2 (triphosphate)
wherein
   R5 and R6 are independently
      —C(R7)(R8)C(O)OR9;
      wherein
         R7 and R8 are independently
         hydrogen,
         Alkyl,
         aryl(C1-C6)alkyl,
         or
         phenyl;
         R9 is independently C1-C6 alkyl or aryl(C1-C6)alkyl;
   and
Ar is independently selected from the group consisting of
   phenyl
   1-naphthyl
   2-naphthyl

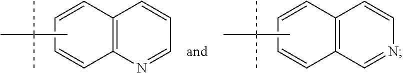

and the tautomers and the pharmaceutically acceptable salts thereof.

The anomeric linkage between the sugar moiety and the aglycone of Formula I may be of either β-D or α-D configuration.

In each case, the above compounds are provided as racemic phosphorous compounds along with the phosphorus diastereomers.

In accordance with the present invention there are provided the following specific embodiments of the above compounds:

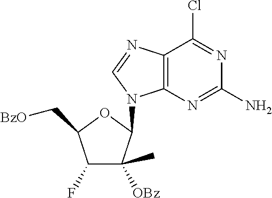

((2R,3R,4S,5R)-5-(2-Amino-6-chloro-9H-purin-9-yl)-4-(benzoyloxy)-3-fluoro-4-methyltetrahydrofuran-2-yl)methyl benzoate

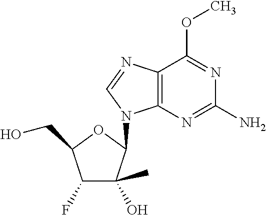 [SMILES: COc1nc(N)nc2c1ncn2[C@@H]1O[C@H](CO)[C@@H](F)[C@@]1(C)O]

(2R,3S,4R,5R)-2-(2-Amino-6-methoxy-9H-purin-9-yl)-4-fluoro-5-(hydroxymethyl)-3-methyltetrahydrofuran-3-ol

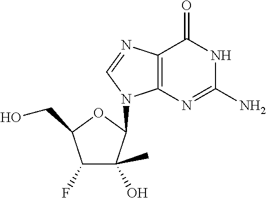 [SMILES: C[C@@]1(O)[C@H](F)[C@@H](CO)O[C@H]1n1cnc2c(=O)[nH]c(N)nc21]

2-Amino-9-((2R,3S,4R,5R)-4-fluoro-3-hydroxy-5-(hydroxymethyl)-3-methyltetrahydrofuran-2-yl)-1H-purin-6(9H)-one

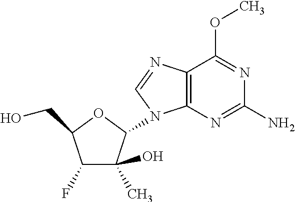 [SMILES: COc1nc(N)nc2c1ncn2[C@H]1O[C@H](CO)[C@@H](F)[C@]1(C)O]

(2S,3R,4R,5R)-2-(2-amino-6-methoxy-9H-purin-9-yl)-4-fluoro-5-(hydroxymethyl)-3-methyltetrahydrofuran-3-ol

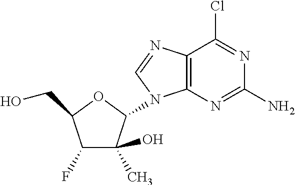 [SMILES: C[C@]1(O)[C@H](F)[C@@H](CO)O[C@@H]1n1cnc2c(Cl)nc(N)nc21]

(2S,3R,4R,5R)-2-(2-amino-6-chloro-9H-purin-9-yl)-4-fluoro-5-(hydroxymethyl)-3-methyltetrahydrofuran-3-ol

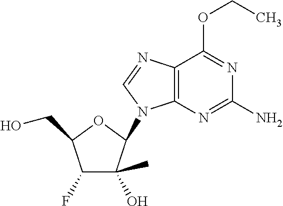 [SMILES: CCOc1nc(N)nc2c1ncn2[C@@H]1O[C@H](CO)[C@@H](F)[C@@]1(C)O]

(2R,3S,4R,5R)-2-(2-Amino-6-ethoxy-9H-purin-9-yl)-4-fluoro-5-(hydroxymethyl)-3-methyltetrahydrofuran-3-ol

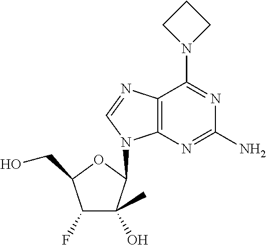 [SMILES: C[C@@]1(O)[C@H](F)[C@@H](CO)O[C@H]1n1cnc2c(N3CCC3)nc(N)nc21]

(3S,4R,5R)-2-(2-Amino-6-(azetidin-1-yl)-9H-purin-9-yl)-4-fluoro-5-(hydroxymethyl)-3-methyltetrahydrofuran-3-ol

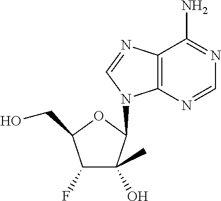 [SMILES: C[C@@]1(O)[C@H](F)[C@@H](CO)O[C@H]1n1cnc2c(N)ncnc21]

(2R,3S,4R,5R)-2-(6-Amino-9H-purin-9-yl)-4-fluoro-5-(hydroxymethyl)-3-methyltetrahydrofuran-3-ol

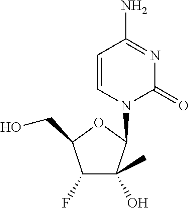 [SMILES: C[C@@]1(O)[C@H](F)[C@@H](CO)O[C@H]1n1ccc(N)nc1=O]

4-Amino-1-((2R,3S,4R,5R)-4-fluoro-3-hydroxy-5-(hydroxymethyl)-3-methyltetrahydrofuran-2-yl)pyrimidin-2(1H)-one

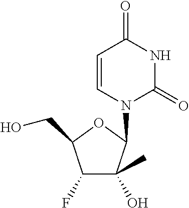 [SMILES: C[C@@]1(O)[C@H](F)[C@@H](CO)O[C@H]1n1ccc(=O)[nH]c1=O]

1-((2R,3S,4R,5R)-4-Fluoro-3-hydroxy-5-(hydroxymethyl)-3-methyltetrahydrofuran-2-yl)pyrimidine-2,4(1H,3H)-dione

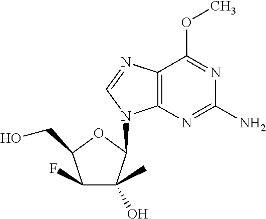 [SMILES: COc1nc(N)nc2c1ncn2[C@@H]1O[C@H](CO)[C@H](F)[C@@]1(C)O]

(2R,3S,4S,5R)-2-(2-Amino-6-methoxy-9H-purin-9-yl)-4-fluoro-5-(hydroxymethyl)-3-methyltetrahydrofuran-3-ol

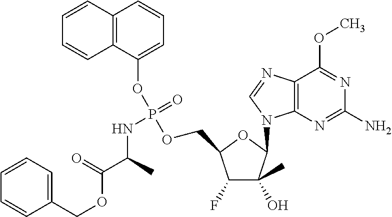 [SMILES: COc1nc(N)nc2c1ncn2[C@@H]1O[C@H](COP(=O)(N[C@@H](C)C(=O)OCc2ccccc2)Oc2cccc3ccccc23)[C@@H](F)[C@@]1(C)O]

(2S)-Benzyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

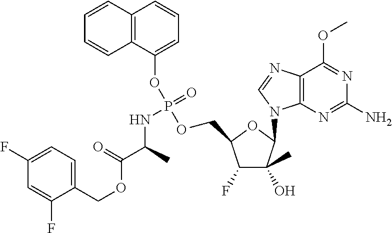 [SMILES: COc1nc(N)nc2c1ncn2[C@@H]1O[C@H](COP(=O)(N[C@@H](C)C(=O)OCc2ccc(F)cc2F)Oc2cccc3ccccc23)[C@@H](F)[C@@]1(C)O]

(2S)-2,4-Difluorobenzyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

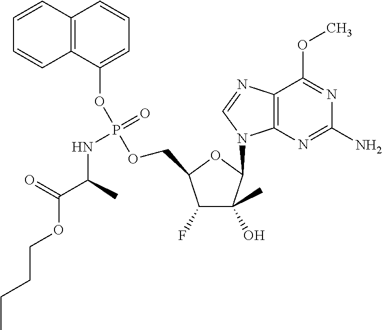 [SMILES: CCCCOC(=O)[C@H](C)NP(=O)(OC[C@H]1O[C@@H](n2cnc3c(OC)nc(N)nc32)[C@](C)(O)[C@@H]1F)Oc1cccc2ccccc12]

(2S)-Butyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

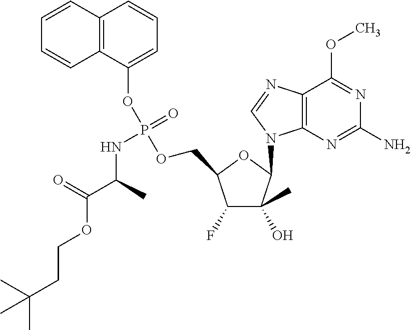 [SMILES: COc1nc(N)nc2c1ncn2[C@@H]1O[C@H](COP(=O)(N[C@@H](C)C(=O)OCCC(C)(C)C)Oc2cccc3ccccc23)[C@@H](F)[C@@]1(C)O]

(2S)-3,3-Dimethylbutyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

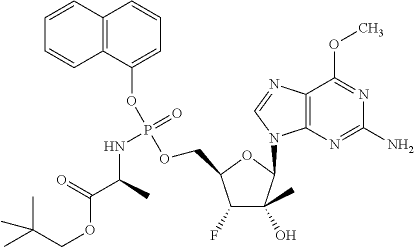 [SMILES: COc1nc(N)nc2c1ncn2[C@@H]1O[C@H](COP(=O)(N[C@@H](C)C(=O)OCC(C)(C)C)Oc2cccc3ccccc23)[C@@H](F)[C@@]1(C)O]

(2S)-Neopentyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

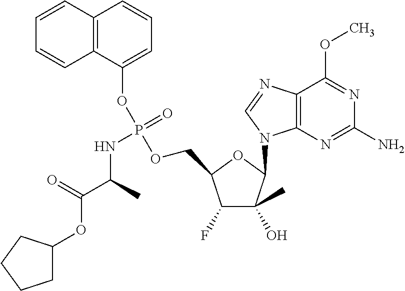 [SMILES: COc1nc(N)nc2c1ncn2[C@@H]1O[C@H](COP(=O)(N[C@@H](C)C(=O)OC2CCCC2)Oc2cccc3ccccc23)[C@@H](F)[C@@]1(C)O]

(2S)-Cyclopentyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

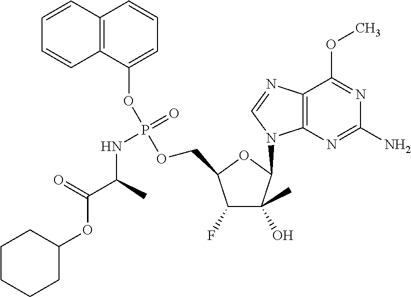 [SMILES: COc1nc(N)nc2c1ncn2[C@@H]1O[C@H](COP(=O)(N[C@@H](C)C(=O)OC2CCCCC2)Oc2cccc3ccccc23)[C@@H](F)[C@@]1(C)O]

(2S)-Cyclohexyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

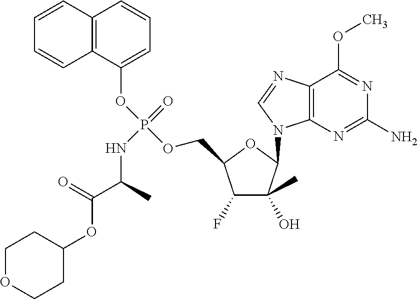 [SMILES: COc1nc(N)nc2c1ncn2[C@@H]1O[C@H](COP(=O)(N[C@@H](C)C(=O)OC2CCOCC2)Oc2cccc3ccccc23)[C@@H](F)[C@@]1(C)O]

(2S)-Tetrahydro-2H-pyran-4-yl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

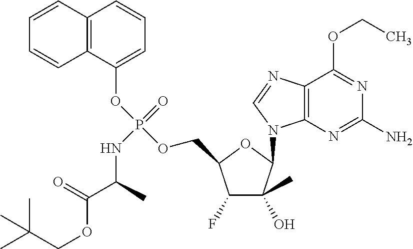 [SMILES: CCOc1nc(N)nc2c1ncn2[C@@H]1O[C@H](COP(=O)(N[C@@H](C)C(=O)OCC(C)(C)C)Oc2cccc3ccccc23)[C@@H](F)[C@@]1(C)O]

(2S)-Neopentyl 2-((((2R,3R,4S)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate

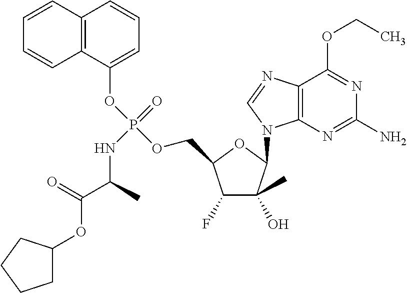 [SMILES: CCOc1nc(N)nc2c1ncn2[C@@H]1O[C@H](COP(=O)(N[C@@H](C)C(=O)OC2CCCC2)Oc2cccc3ccccc23)[C@@H](F)[C@@]1(C)O]

(2S)-Cyclopentyl 2-((((2R,3R,4S)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate

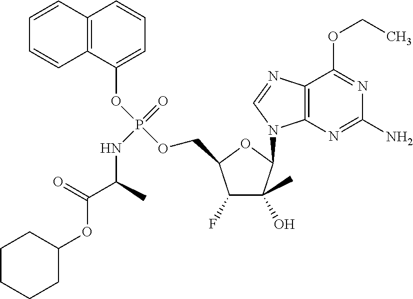 [SMILES: CCOc1nc(N)nc2c1ncn2[C@@H]1O[C@H](COP(=O)(N[C@@H](C)C(=O)OC2CCCCC2)Oc2cccc3ccccc23)[C@@H](F)[C@@]1(C)O]

(2S)-Cyclohexyl 2-((((2R,3R,4S)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate

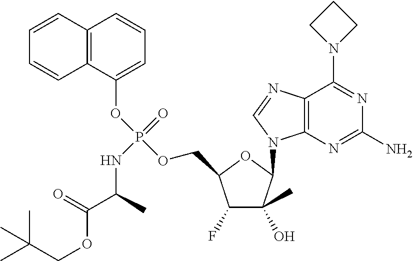 [SMILES: C[C@H](NP(=O)(OC[C@H]1O[C@@H](n2cnc3c(N4CCC4)nc(N)nc32)[C@](C)(O)[C@@H]1F)Oc1cccc2ccccc12)C(=O)OCC(C)(C)C]

(2S)-Neopentyl 2-((((2R,3R,4S)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate

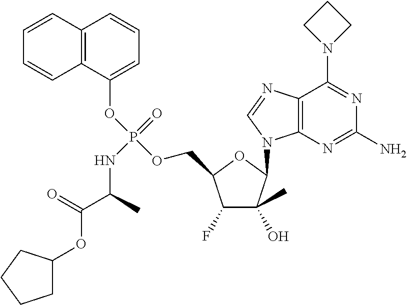 [SMILES: C[C@H](NP(=O)(OC[C@H]1O[C@@H](n2cnc3c(N4CCC4)nc(N)nc32)[C@](C)(O)[C@@H]1F)Oc1cccc2ccccc12)C(=O)OC1CCCC1]

(2S)-Cyclopentyl 2-((((2R,3R,4S)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate

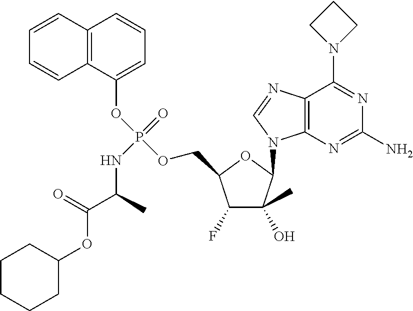 [SMILES: C[C@H](NP(=O)(OC[C@H]1O[C@@H](n2cnc3c(N4CCC4)nc(N)nc32)[C@](C)(O)[C@@H]1F)Oc1cccc2ccccc12)C(=O)OC1CCCCC1]

(2S)-Cyclohexyl 2-((((2R,3R,4S)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate

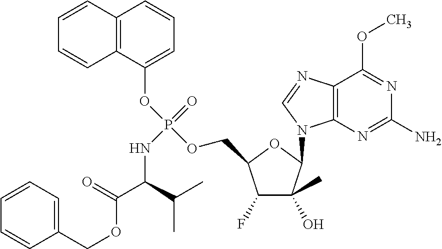 [SMILES: COc1nc(N)nc2c1ncn2[C@@H]1O[C@H](COP(=O)(N[C@H](C(=O)OCc2ccccc2)C(C)C)Oc2cccc3ccccc23)[C@@H](F)[C@@]1(C)O]

(2S)-Benzyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate

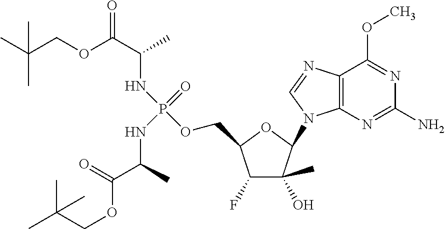 [SMILES: COc1nc(N)nc2c1ncn2[C@@H]1O[C@H](COP(=O)(N[C@@H](C)C(=O)OCC(C)(C)C)N[C@@H](C)C(=O)OCC(C)(C)C)[C@@H](F)[C@@]1(C)O]

(2S,2′S)-Neopentyl 2,2′-((((2R,3R,4S,5R)-5-(2-amino-6-methoxy-9H-purin-9-yl)-3-fluoro-4-hydroxy-4-methyltetrahydrofuran-2-yl)methoxy)phosphoryl)bis(azanediyl)dipropanoate

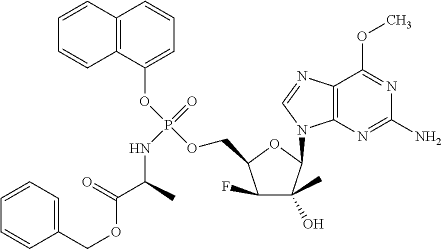 [SMILES: COc1nc(N)nc2c1ncn2[C@@H]1O[C@H](COP(=O)(N[C@@H](C)C(=O)OCc2ccccc2)Oc2cccc3ccccc23)[C@H](F)[C@@]1(C)O]

(2S)-Benzyl 2-((((2R,3S,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

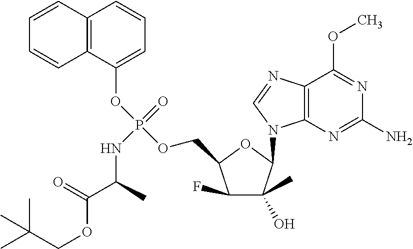 [SMILES: COc1nc(N)nc2c1ncn2[C@@H]1O[C@H](COP(=O)(N[C@@H](C)C(=O)OCC(C)(C)C)Oc2cccc3ccccc23)[C@H](F)[C@@]1(C)O]

(2S)-Neopentyl 2-((((2R,3S,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

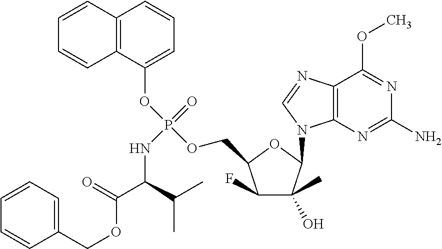 [SMILES: COc1nc(N)nc2c1ncn2[C@@H]1O[C@H](COP(=O)(N[C@H](C(=O)OCc2ccccc2)C(C)C)Oc2cccc3ccccc23)[C@H](F)[C@@]1(C)O]

(2S)-Benzyl 2-((((2R,3S,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate

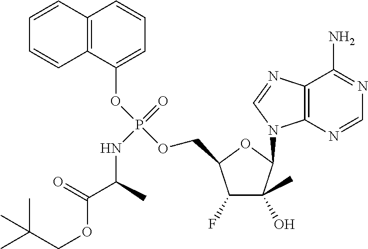 [SMILES: C[C@H](NP(=O)(OC[C@H]1O[C@@H](n2cnc3c(N)ncnc32)[C@](C)(O)[C@@H]1F)Oc1cccc2ccccc12)C(=O)OCC(C)(C)C]

(2S)-Neopentyl 2-((((2R,3R,4S,5R)-5-(6-amino-9H-purin-9-yl)-3-fluoro-4-hydroxy-4-methyltetrahydrofuran-2-yl)methoxy)(naphthalen-1-yloxy)phosphorylamino)propanoate

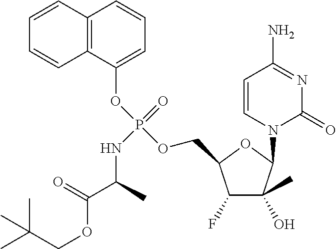 [SMILES: C[C@H](NP(=O)(OC[C@H]1O[C@@H](n2ccc(N)nc2=O)[C@](C)(O)[C@@H]1F)Oc1cccc2ccccc12)C(=O)OCC(C)(C)C]

(2S)-Neopentyl 2-((((2R,3R,4S,5R)-5-(4-amino-2-oxopyrimidin-1(2H)-yl)-3-fluoro-4-hydroxy-4-methyltetrahydrofuran-2-yl)methoxy)(naphthalen-1-yloxy)phosphorylamino)propanoate

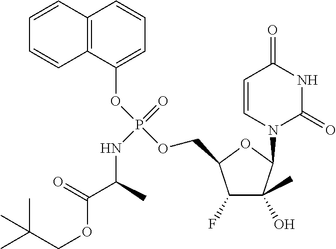 [SMILES: C[C@H](NP(=O)(OC[C@H]1O[C@@H](n2ccc(=O)[nH]c2=O)[C@](C)(O)[C@@H]1F)Oc1cccc2ccccc12)C(=O)OCC(C)(C)C]

(2S)-Neopentyl 2-((((2R,3R,4S,5R)-5-(2,4-dioxo-3,4-dihydropyrimidin-1(2H)-yl)-3-fluoro-4-hydroxy-4-methyltetrahydrofuran-2-yl)methoxy)(naphthalen-1-yloxy)phosphorylamino)propanoate

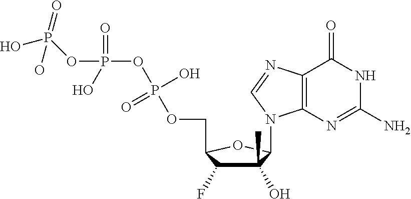 [SMILES: C[C@]1(O)[C@H](F)[C@@H](COP(=O)(O)OP(=O)(O)OP(=O)(O)O)O[C@H]1n1cnc2c(=O)[nH]c(N)nc21]

((2R,5R)-5-(2-Amino-6-oxo-1H-purin-9(6H)-yl)-3-fluoro-4-hydroxy-4-methyltetrahydrofuran-2-yl)methyl tetrahydrogen triphosphate

In each case with regard to the foregoing specific compounds, it is contemplated that the invention will include phosphorus diastereomers thereof.

Compounds according to the present invention have surprisingly been found to have excellent anti-viral activity. In particular, compounds according to the present invention have been found to have excellent potency with respect to hepatitis C virus.

### DEFINITIONS

As used herein, the term “alkyl” refers to a straight or branched saturated monovalent cyclic or acyclic hydrocarbon radical, having the number of carbon atoms as indicated (or where not indicated, an acyclic alkyl group preferably has 1-20, more preferably 1-6, most preferably 1-4 carbon atoms and a cyclic alkyl group preferably has 3-20, more preferably 3-10, most preferably 3-7 carbon atoms), optionally substituted with one, two, three or more substituents independently selected from the group set out above. By way of non-limiting examples, suitable alkyl groups include methyl, ethyl, propyl, butyl, pentyl, hexyl, octyl, nonyl, isopropyl, 2-butyl, cyclopropyl, cyclohexyl, cyclopentyl and dodecyl. The term “C3-C8cycloalkyl” refers to cyclic alkyl group comprising from about 3 to about 8 C atoms. The term “C3-C8cycloalkyl-alkyl” refers to an acyclic alkyl group substituted by a cyclic alkyl group comprising from about 3 to about 8 C atoms.

As use herein, the term “alkoxy” or the term “alkyloxy” refers to the group alkyl-O—, where alkyl is as defined above and where the alkyl moiety may optionally be substituted by one, two, three or more substituents as set out above for alkyl. By way of non-limiting examples, suitable alkoxy groups include methoxy, ethoxy, n-propoxy, iso-propoxy, n-butoxy, tert-butoxy, sec-butoxy, n-pentoxy, n-hexoxy and 1,2-dimethylbutoxy. The term “cycloalkyloxy” refers to the group cyclicalkyl-O—, where cyclicalkyl is as defined above and where the cyclicalkyl moiety may be optionally substituted by one, two, three or more substituents as set out above for alkyl.

As used herein, the term “cycloalkylaryl” refers to an aryl group having a cyclic alkyl substituent. Binding is through the aryl group. The cycloalkyl moiety and the aryl moiety are as defined herein with respect to the definitions of cycloalkyl and aryl, respectively.

As used herein, the terms “aryl(C1-C3)alkyl” and “aryl(C1-C6)alkyl” refer to a C1-C3 alkyl group or a C1-C6 alkyl group, respectively, substituted at any carbon by an aryl group. Binding to the rest of the molecule is through the alkyl group. The aryl moiety and the alkyl moiety are as defined herein with respect to the definitions of aryl and alkyl. The aryl group may be substituted. By way of non-limiting examples, suitable aryl(C1-C3)alkyl groups include benzyl, 1-phenylethyl, 3-phenylpropyl, 4-chlorobenzyl, 4-fluorobenzyl, 2,4-difluorobenzyl, and the like. By way of non-limiting examples, suitable aryl(C1-C6)alkyl groups include the aryl(C1-C3)alkyl groups described above as well as 1-phenylbutyl, 3-phenylpentyl, 6-(4-chlorophenyl)hexyl, 4(4-fluorophenyl)pentyl, 5-(2,4-difluorophenyl)hexyl, and the like.

A cycloalkyl moiety and the aryl moiety may each be optionally substituted by one, two, three or more substituents as set out herein with regard to the definitions of alkyl and aryl, respectively.

As used herein the term “aryl” refers to a monovalent unsaturated aromatic carbocyclic radical having one, two, three, four, five or six rings, preferably one, two or three rings, which may be fused or bicyclic. An aryl group may optionally be substituted by one, two, three or more substituents as set out above with respect to optional substituents that may be present on the group Ar. Preferred aryl groups are: an aromatic monocyclic ring containing 6 carbon atoms; an aromatic bicyclic or fused ring system containing 7, 8, 9 or 10 carbon atoms; or an aromatic tricyclic ring system containing 10, 11, 12, 13 or 14 carbon atoms. Non-limiting examples of aryl include phenyl and naphthyl. These compounds may include substituent groups, preferably those substituent groups independently selected from hydroxy (—OH), acyl (R′—C(═O)), acyloxy (R′—C(O)—O—), nitro (—NO2), amino (—NH2), carboxyl (—COOH), cyano (—CN), C1-C6monoalkylamino, C1-C 6dialkylamino, thiol, chloro, bromo, fluoro, iodo, SO3H, —SH, —SR′, wherein R′ is independently selected from halo, C1-C6alkoxy, and C1-C6alkyl.

When a radical is drawn as a structure, e.g.,

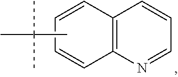

the linking bond between the radical and the rest of the molecule is depicted in the drawing by the fragment

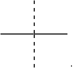

The point of attachment on the radical is at any available position of the ring into which the linking bond is drawn.

The compounds of this invention may contain one or more asymmetric centers, depending upon the location and nature of the various substituents desired. Asymmetric carbon atoms or phosphorous atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form, and a substituent on a double bond may be present in either Z or E form. It is intended that all such configurations (including enantiomers and diastereomers) are included within the scope of the present invention. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like, also well-known in the art and exemplified the experimental examples below. Separated, pure or partially purified isomers or racemic mixtures of the compounds of this invention are also included within the scope of the present invention.

Preferred compounds are those with the absolute configuration of the compound of this invention which produces the more desirable biological activity.

The term “pharmaceutically acceptable salt” refers to pharmaceutically acceptable salts of a compound, which salts are derived from a variety of organic and inorganic counter ions well known in the art and include, by way of example only and not limited to, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “pharmaceutically acceptable partial salts” is included in the term “pharmaceutically acceptable salts” and refers to compounds having a substituent capable of having more than one group form a salt but less than the maximum amount of such groups actually form a salt. For example, a diphospho group can form a plurality of salts and, if only partially ionized, the resulting group is sometimes referred to herein as a partial salt. It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl- (substituted aryl)-substituted aryl. Similarly, it is understood that the above definitions are not intended to include impermissible substitution patterns (e.g., methyl substituted with 5 fluoro groups or a hydroxyl group pendent to a carbon atom of an ethenylic or acetylenic unsaturation). Such impermissible substitution patterns are well known to the skilled artisan.

When a compound is depicted as a charged species, e.g.,

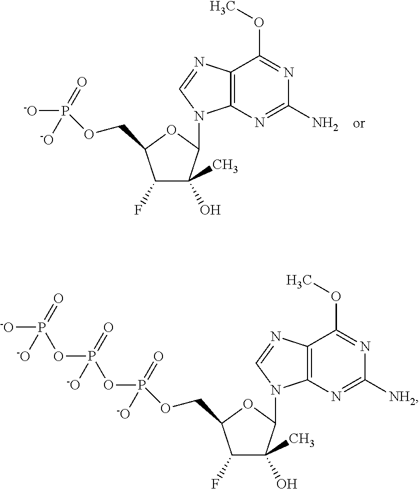

it is to be understood that the compound is in a salt form, and exists together with the appropriate number of pharmaceutically acceptable counter ions, as described above, as required to produce a neutral species.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). Specifically, the compounds of this invention may be prepared by various methods known in the art of organic chemistry in general and nucleoside and nucleotide analogue synthesis in particular. General reviews of the preparation of nucleoside and nucleotide analogues include 1) Michelson A. M., “The Chemistry of Nucleosides and Nucleotides,” Academic Press, New York, 1963; 2) Goodman L., “Basic Principles in Nucleic Acid Chemistry,” Academic Press, New York, 1974, vol. 1, Ch. 2; and 3) “Synthetic Procedures in Nucleic Acid Chemistry,” Eds. Zorbach W. & Tipson R., Wiley, New York, 1973, vol. 1 & 2.

Strategies available for synthesis of compounds of this invention are illustrated in the synthetic schemes below.

Reaction Scheme 1 illustrates a general method for the preparation of ((2R,3R,4S)-5-acetoxy-4-(benzoyloxy)-3-fluoro-4-methyltetrahydrofuran-2-yl)methyl benzoate

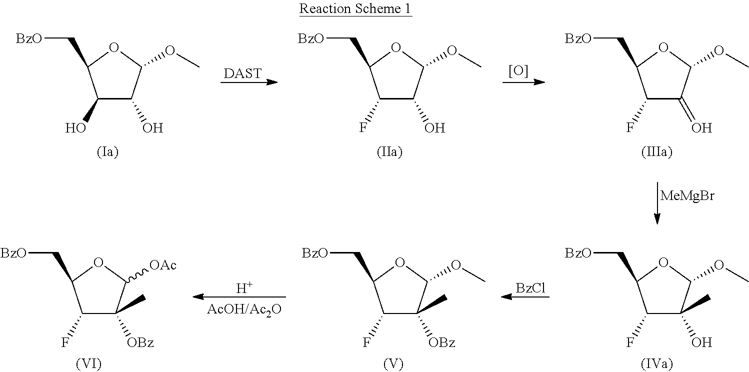

Compound (Ia) in Scheme 1 can be synthesized in two steps from the known 1,2-isopropylidene D-xylose with a regioselective benzoylation and subsequent removal of the 1,2-isopropylidene in methanol with acid or by other conditions familiar to one skilled in the art. The resulting ((2R,3R,4R,5S)-3,4-dihydroxy-5-methoxytetrahydrofuran-2-yl)methyl benzoate can be fluorinated with a variety of known fluorinating agents, such as DAST, in a variety on aprotic non-polar organic solvents such as THF. Both possible fluoro groups are obtained but the ((2R,3S,4S,5S)-3-fluoro-4-hydroxy-5-methoxytetrahydrofuran-2-yl)methyl benzoate derivative (IIa) is the major product. Compound (IIa) can be oxidized under a variety of conditions known to one skilled in the art, including with Dess-Martin reagent to give compound (IIIa). Alkylation of the ketone of ((2R,3R,5S)-3-fluoro-5-methoxy-4-oxotetrahydrofuran-2-yl)methyl benzoate (IIIa) with an alkylating agent compatible with the presence of the ester protecting group, leads to ((2R,3R,4S,5S)-3-fluoro-4-hydroxy-5-methoxy-4-methyltetrahydrofuran-2-yl)methyl benzoate (IVa), which is then benzoylated to give compound (V) and then converted to the anomeric acetate (VI) using standard acid catalyzed conditions.

Reaction Scheme 2 illustrates a general method for coupling the activated fluoro sugar (VI) to a variety of purine derivatives.

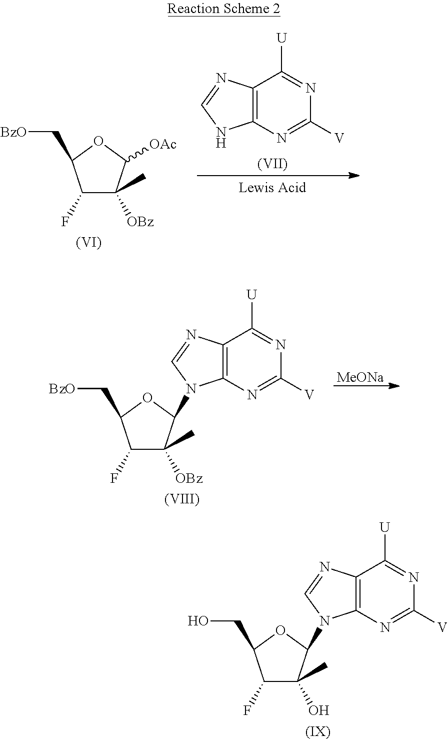

Using standard Vorbruggen conditions, a variety of substituted purine derivatives (VII) can be coupled to ((2R,3R,4S)-5-acetoxy-4-(benzoyloxy)-3-fluoro-4-methyltetrahydrofuran-2-yl)methyl benzoate (VI), in aprotic solvents including but not limited to acetonitrile, dichloromethane and THF. The resulting nucleosides (VIII) can then be de-benzoylated using standard procedures such as ammonia in an alcoholic solvent or sodium methoxide in methanol. At the same time modifications of the groups U and V can be effected. For instance if U is a chloro group it can be converted to a methoxy group by treatment with sodium methoxide. These reactions can be done separately or in one pot to provide purine nucleosides of general structure (IX).

Reaction Scheme 3 illustrates a general method for coupling ((2R,3R,4S)-5-acetoxy-4-(benzoyloxy)-3-fluoro-4-methyltetrahydrofuran-2-yl)methyl benzoate (VI) with pyrimidin-2-one derivatives (X).

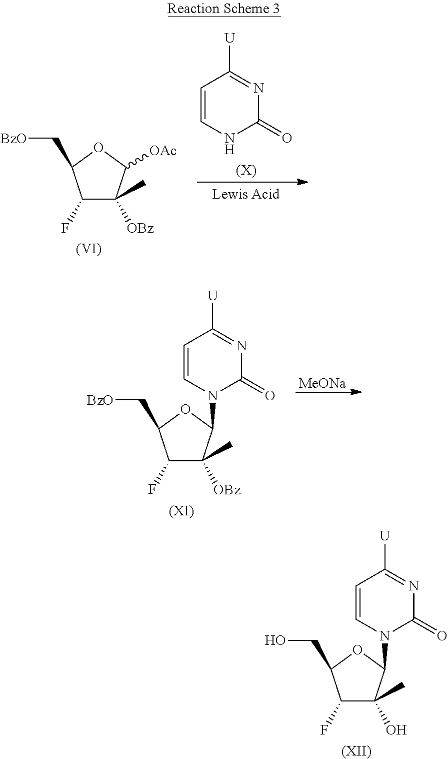

Compound (VI), ((2R,3R,4S)-5-acetoxy-4-(benzoyloxy)-3-fluoro-4-methyltetrahydrofuran-2-yl)methyl benzoate, can be coupled to various pyrimidin-2-one heterocycles such as cytidine or uracil using an aprotic solvent such as acetonitrile, dichloroethane, dichloromethane or THF along with a Lewis acid such as TMS triflate, or SnCl4. The heterocycle (X) can be per-silylated using N,O-bistrimethylsilylacetamide (BSA) or some other silylating agent such as TMS triflate or TMS chloride. The protected nucleoside derivatives (XI), can then be deprotected using standard nucleoside chemistry. For example compound (XI) can be treated with sodium methoxide in methanol at ambient temperature for 36 hours. Alternatively, many other debenzoylating procedures may be used including alcoholic ammonia.

Reaction Scheme 4 illustrates a general method for preparing (3S,4S,5R)-5-(benzyloxymethyl)-4-fluoro-3-methyltetrahydrofuran-2,3-diyl dibenzoate (XXIII) starting from the ribono lactone XIV.

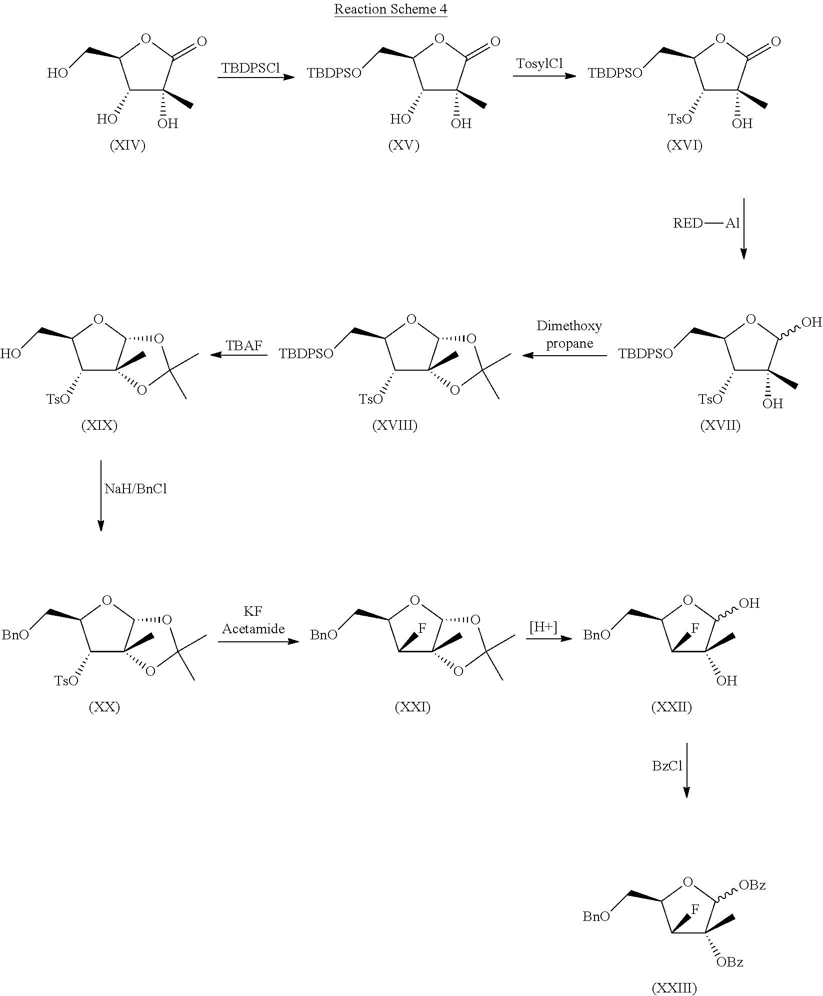

Commercially available ribonolactone, (3R,4R,5R)-3,4-dihydroxy-5-(hydroxymethyl)-3-methyldihydrofuran-2(3H)-one, (XIV) can be protected at the primary hydroxyl using standard silylating conditions and selecting from a variety of silylating agents such as TBDPSCl, TPSCl, or TBDMSCl, and preferably tert-butyldimethylsilyl chloride. The secondary alcohol can then be converted to the tosyl ester, using standard conditions. Reduction to the ribose, (2R,3R,4R)-2-((tert-butyldiphenylsilyloxy)methyl)-4,5-dihydroxy-4-methyltetrahydrofuran-3-yl 4-methylbenzenesulfonate can be accomplished with a variety of reducing agents, and preferably with RED-AL. Protection of the anomeric alcohol and the teriary alcohol can be accomplished with 2,2-dimethoxypropane, to give the isopropylidene, (XVIII). The primary alcohol protecting group can be switched to the more stable benzyl ether using standard protecting group chemistry, to give compound (XX), which can then be treated with a source of nucleophilic fluoride such as sodium fluoride in a high boiling solvent such as N-methyl morpholine or preferably acetamide to give compound (XXI). (3aR,5R,6S,6aS)-5-(Benzyloxymethyl)-6-fluoro-2,2,6a-trimethyltetrahydrofuro[2,3-d][1,3]dioxole can be deprotected to the diol (XXII) using standard isopropylidene deprotection chemistry, and subsequently dibenzoylated with benzoyl chloride under basic conditions, trapping the fluoro sugar as the activated furanoside, (XVIII), which can be used in standard nucleoside coupling reactions with a variety of pyrimidine and purine heterocycles.

Reaction Scheme 5 illustrates a general method for the preparation of Compound (XVIII) with Purine Heterocycle (XXIV)

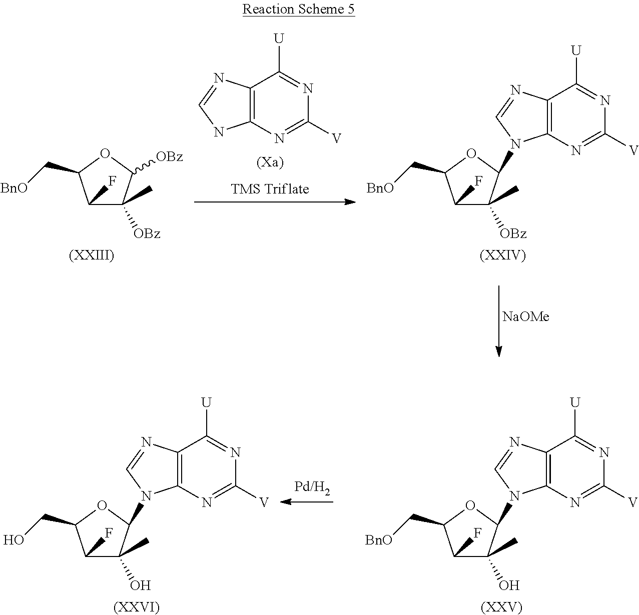

The activated sugar derivative (3S,4S,5R)-5-(benzyloxymethyl)-4-fluoro-3-methyltetrahydrofuran-2,3-diyl dibenzoate (XXIII) can be coupled under standard nucleoside coupling conditions to purine derivatives (Xa) using aprotic polar or non-polar solvents such as acetonitrile, dichloromethane, or 1,2-dichloroethane and a lewis acid such as TMS triflate or SnCl4. The coupled product (XXIV) can be de-benzoylated with sodium methoxide or methanolic ammonia to give the partially deprotected product (XXV). Removal of the benzyl protecting group can be preformed under reducing conditions to give compound (XXVI).

Schemes 1-5 above have described general and successful methods for preparation of novel fluoro sugar derivatives and ultimately fluorinated nucleoside derivatives. The nucleoside derivatives can then be converted into a variety of phosphorylated derivatives including phosphoramidates and phosphordiamidates using procedures described in the schemes below.

Reaction Scheme 6 illustrates a general method for preparing phosphoramidates of general structure (XXXVI)

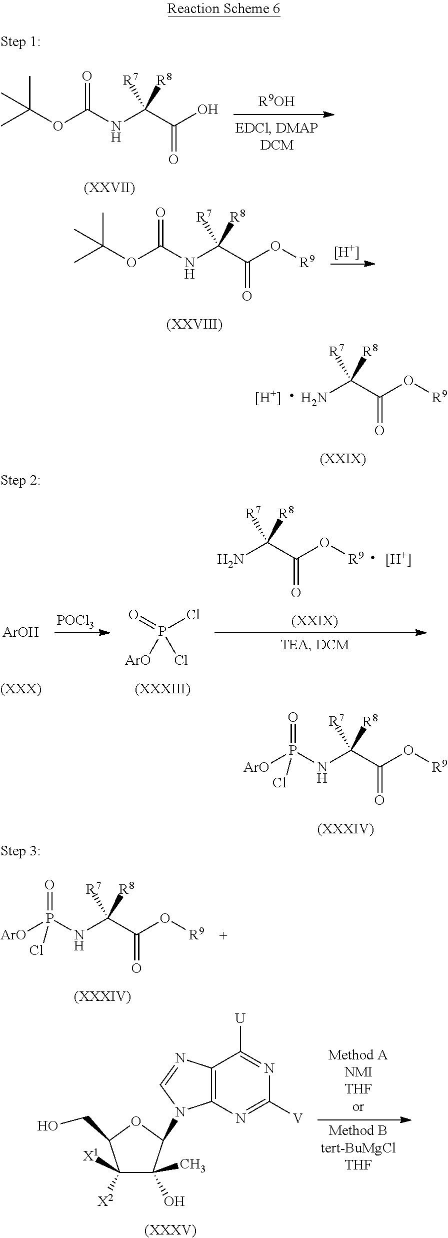

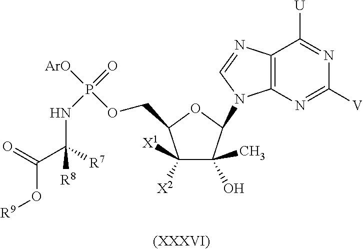

In step 1 of Reaction Scheme 4, a protected amino acid of general formula (XXXVII) is esterified with an alcohol of formula R9OH, facilitated by addition of such reagents as EDCI and DMAP, and carried out in an inert solvent such as dichloromethane, to produce the compound of formula (XXVIII). Alternatively, many other commonly used ester forming reagents such as DCC/DMAP, trifluoroacetic anhydride, N,N′-carbonyldiimidazole and PPh3/CCl4 can be used. Removal of the BOC protecting group from compound (XXVIII) and conversion to a salt of formula (XXIX) is carried out by its reaction with an organic acid such as PTSA in a suitable solvent such as ethyl acetate. Alternatively the BOC group can be removed with other acids such as trifluoroacetic acid and hydrochloric acid (HCl) which provide the corresponding TFA or HCl salts.

In Step 2 of Reaction Scheme 6, the aryl chloroamidate of formula (XXXIII) is prepared by reaction of the hydroxylated aryl compound of formula (XXX) with phosphorous oxychloride in the presence of a non-nucleophilic base such as triethylamine, to provide the intermediate of formula (XXXIII). This reaction can be run in an aprotic solvent such as DCM, ether or MTBE and at low temperatures, preferably 0° C. to −78° C. and preferably at −25° C. In addition to triethyl amine, many other non-nucleophilic bases can be used such as DIEA or DBU. The product of this reaction can be used directly in the next reaction, or the amine salts generated in the reaction, such as triethyl ammonium hydrochloride, can be filtered off prior to the next step. The reaction should be protected from moisture at all times, as the phosphorodichloridate product, is very moisture sensitive. Subsequent reaction of (XXXIII) with the amino ester salt of formula (XXIX) is carried out in the presence of a base to produce the compound of formula (XXXIV). This reaction can be done at 0° C. to −78° C., and preferably at −25° C. A variety of non-polar aprotic solvents may be used such as ether, MTBE, and DCM. The base may be selected from a wide variety on non-nucleophilic organic amines such as, but not limited to, TEA, DIEA, or DBU. This reaction must be protected from moisture at all times. Upon completion of the reaction it is critical to remove the corresponding organic amine salts, such as triethyl ammonium hydrochloride, or triethyl ammonium p-toluene sulfonic acid. This can be accomplished by concentrating the reaction mixture and precipitating the salt with EtOAc and Hexanes and filtering them off, or by passing the crude product through a silica gel plug. Removal of solvents during the work up must be done at temperatures at or below 25° C. to avoid decomposition of the phosphorochloridate.

In Step 3, coupling of chloro phosphoramidates of the formula (XXXIV) and fluoro nucleosides of formula (XXXV) to provide phosphoramidates of general formula (XXXVI) can be carried out using an nucleophilic catalyst such as NMI in an inert organic solvent such as THF. Other nucleophilic catalysts such as DMAP, trimethylamine, pyridine, or 4-(pyrrolidin-1-yl)pyridine, can be used as well as other aprotic solvents such as diethyl ether, MTBE chloroform or DCM. These reactions can be carried out between 0° C. and 50° C., and preferably at 25° C. Alternatively a strong non-nucleophilic base, e.g., tert-butyl magnesium chloride can be used in a solvent such as THF, or diethyl ether, or MTBE. Other strong proton selective organic or inorganic bases can be used such as n-butyl lithium, potassium tert-butoxide, 2,4,6-collidine, DBU, or lithium bis(trimethylsilyl)amide. This reaction can be carried out at −78° C. to 40° C. and preferably at 0-25° C.

Reaction Scheme 7 illustrates one particular method for preparing the guanine derivatives (XXXVIII) from the purine O6-methyl derivatives of structure (XXXVII). The resulting guanine nucleosides can be used in the synthesis of phosphoramidates as described in Scheme 6 above, or to make phosphordiamidates as described below.

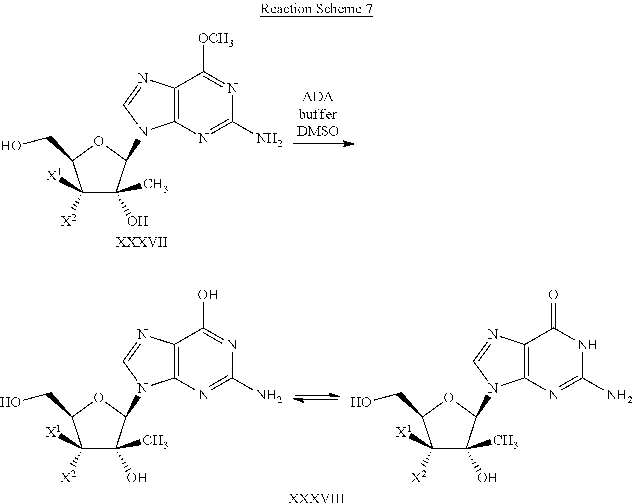

Conversion of the O6 methyl derivatives of formula (XXXVII) to the guanine derivatives of formula (XXXVIII) can be effected using chemistry described in the literature such as M. J. Robins, R. Zou, F. Hansske, S. F. Wnuk Can. J. Chem. (1997), vol 75, pgs 762-767. Alternatively, the O6-methyl group of compound XV can be removed with TMSI either directly or generated in situ with TMSCl and NaI, in the presence of a base such as DMAP or diethylisopropylamine in an aprotic polar solvent such as acetonitrile.

Reaction Scheme 8 illustrates how nucleosides of structure XII can be converted to phosphoramidates of general structure (XXXIX) by reaction with chloroamidate XXXIV.

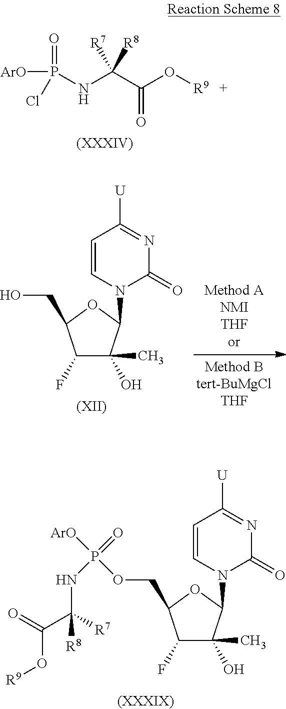

The chlorophosphoramidate of general structure XXXIV can be combined with a pyrimidine nucleoside derivative of general structure XII using either a nucleophilic catalyst such as N-methyl imidazole in a solvent such as THF or other aprotic non-polar or polar solvents such as DCM or alternatively, nucleoside XII can be treated with a base such as tert-butyl magnesium chloride in an aprotic solvent such as THF and subsequently combined with a chlorophosphoramidate of general structure XXXIV.

Reaction Scheme 9 illustrates how the amino acid monophosphate derivatives of formula (XL) are formed by allowing the ester amidate of formula (XXXVI) to react with an esterase such as Carboxypeptidase Y in the presence of a phosphate buffer such as TRIZMA and in the presence of an organic solvent such as acetone. The conversion can be monitored by 31P NMR or HPLC over a period of 5-24 hours.

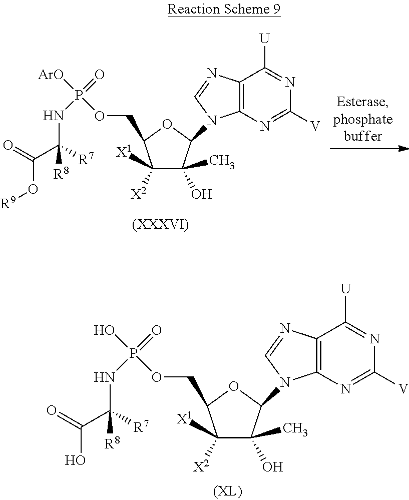

Phosphorous diastereomers of formula (XXXVI), as produced in Reaction Scheme 6, can be separated using chiral chromatography as shown in Reaction Scheme 10.

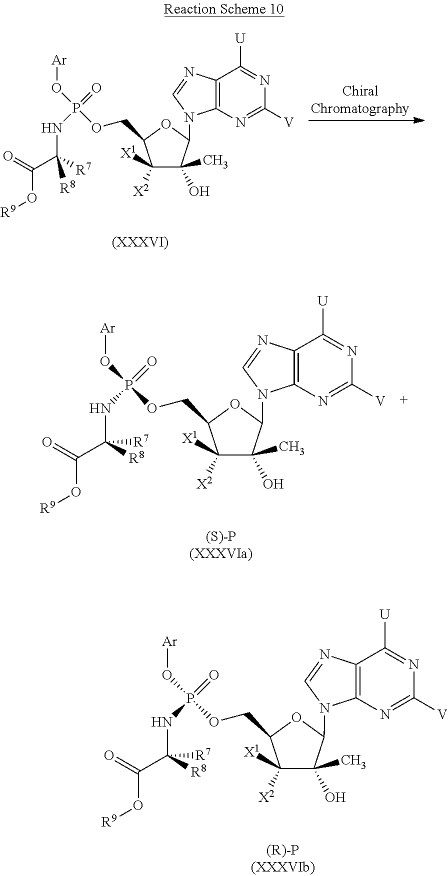

In this Scheme, the chiral chromatography can be performed on a variety of different chiral resins such as CHIRALPAK AD®, CHIRALPAK AS®, CHIRALCEL OD®, CHIRALCEL OJ®, CHIRALCEL OB®, and CHIRALCEL OC® AD-H®, AS-H®, OD-H®, OJ-H®, OB-H® and OC-H®. Alternatively the chiral resin could be selected from the list below:
CHIRALPAK® IA™
CHIRALPAK® IA-3
CHIRALPAK® AD-H
CHIRALPAK® AD
CHIRALPAK® AD-3
CHIRALPAK® AD-3R
CHIRALPAK® AS-H
CHIRALPAK® AS
CHIRALPAK® AY-H
CHIRALPAK® AY
CHIRALPAK® AZ-H
CHIRALPAK® AZ
CHIRALPAK® IB™
CHIRALCEL® OD-H
CHIRALCEL® OD
CHIRALCEL® OD-3
CHIRALCEL® OD-3R
CHIRALCEL® OD-I
CHIRALPAK® IC™
CHIRALPAK® IC-3
CHIRALCEL® OC-H
CHIRALCEL® OC
CHIRALCEL® OA
CHIRALCEL® OB-H
CHIRALCEL® OB
CHIRALCEL® OG
CHIRALCEL® OJ-H
CHIRALCEL® OJ
CHIRALCEL® OF
CHIRALCEL® OK
CHIRALCEL® OZ-H
CHIRALCEL® OZ

Optimally a Chiral Pak AD column can be used with a mixture of 1:1 ethanol:hexanes as the mobile phase. Other solvents such as ethyl acetate, isopropanol, acetonitrile, and methanol can be used as the mobile phase, or other solvents familiar to those skilled in the art.

Reaction Scheme 11 illustrates the synthesis of symmetrical phosphordiamidates from nucleoside derivatives of formula (XXIV).

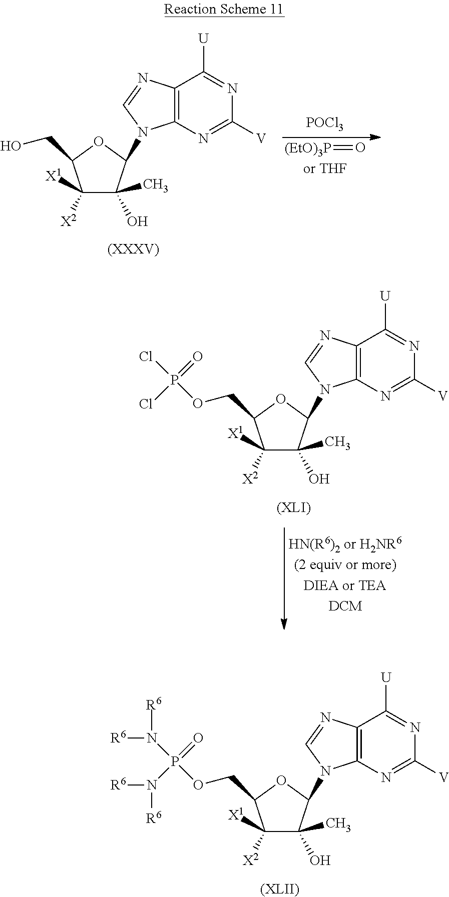

In this scheme, nucleosides of the formula (XXXV), are dissolved in a neutral aprotic solvent such as THF or triethyl phosphate or similar solvent and cooled to below ambient temperature, preferably to 0-5° C. Phosphorus oxychloride (phosphoryl chloride) of high quality is added to the solution with careful protection from moisture. The reaction is stirred for 1-48 h at temperatures from −20° C. to 20° C. and optimally for 24 h at 5° C., forming compounds of the formula (XLI). The solution is diluted with an aprotic solvent, preferably DCM, and a primary or secondary amine, of formula R6NH2 or (R6)2.NH, where each R6 may be the same or different, as defined above for Formula I. These include such primary or secondary amines as the HCl or tosylate salt of an aminoacid ester. The addition of the amine is carried out at reduced temperatures of from about −78° C. to about 5° C. and preferably at about 0° C. This is followed by the addition of a non-nucleophilic base such as a tertiary amine such as triethylamine, or preferably diisopropylethylamine. The solution is stirred for about 1 h to about 10 days at reduced temperatures and preferably at about 5° C. for about 5 days, forming phosphodiamidate (XLII).

Alternatively the nucleoside (XXXV) can be dissolved in a neutral aprotic solvent such as THF or triethyl phosphate or similar solvent, but preferably THF, and a non-nucleophilic base such as a tertiary amine or diisopropylethylamine or preferably triethylamine is added and stirred for a period of about 5 min to about 1 h, preferably about 30 min. The solution is then cooled to about −100° C. to RT, or preferably about −78° C., and phosphorus oxychloride (phosphoryl chloride) of high quality is added slowly to the solution with careful protection from moisture. The reaction is stirred for about 5 min to about 2 h at temperatures from about −100° C. to about 0° C. and optimally for about 30 min at about −78° C., then warmed to ambient temperature for about 5 min to about 2 h, preferably about 30 min forming compound (XLI). The solution is further diluted with an aprotic solvent, preferably DCM, and a primary or secondary amine, such as the HCl or tosylate salt of an aminoacid ester, is added followed by the addition of a non-nucleophilic base such as a tertiary amine preferably triethylamine at reduced temperatures of about −78° C. to about 5° C. and preferably at about −78° C. The solution is warmed to ambient temperature and stirred for about 1 h to about 48 h, preferably about 24 h, forming phosphodiamidate (XLII). The reaction can be worked up using standard methods familiar to one skilled in the art, for example extraction with a sodium chloride solution, drying with sodium sulfate and purification by silica gel chromatography. Changes to this procedure including solvent switches and optimization of the temperature, familiar to those skilled in the art of organic chemistry would be anticipated.

Reaction Scheme 12 illustrates a general method of preparation of asymmetrical phosphoramidates.

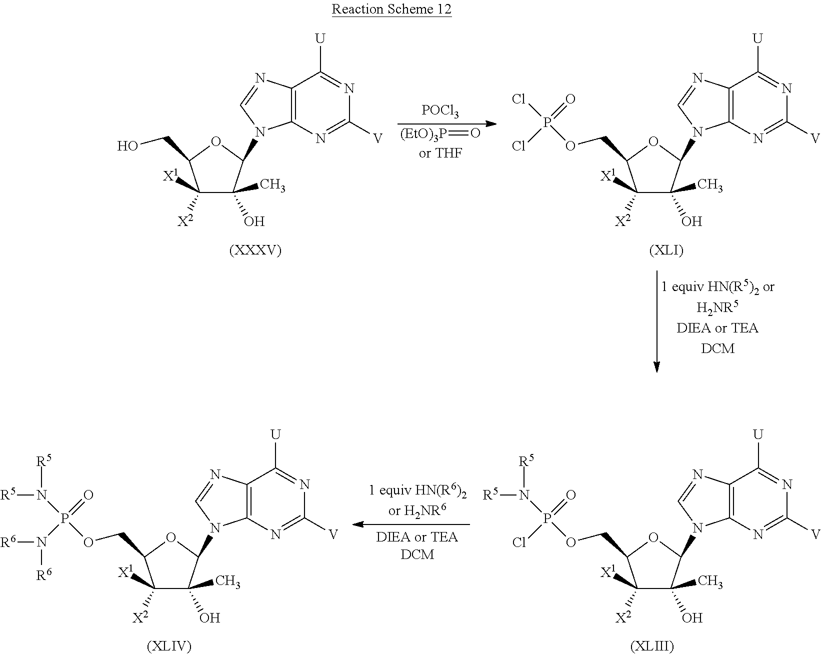

In this scheme a general method for synthesizing asymmetrical phosphodiamidates is described. The nucleoside (XXXV) can be dissolved in a neutral aprotic solvent such as THF or triethyl phosphate or similar solvent, but preferably THF, and a non-nucleophilic base such as a tertiary amine or diisopropylethylamine or preferably triethylamine is added and stirred for a period of about 5 min to about 1 h, preferably about 30 min. The solution is then cooled to about −100° C. to rt, or preferably about −78° C., and phosphorus oxychloride (phosphoryl chloride) of high quality is added slowly to the solution with careful protection from moisture. The reaction is stirred for about 5 min to about 2 h at temperatures ranging from about −100° C. to about 0° C. and optimally for about 30 min at about −78° C., then warmed to ambient temperature for about 5 min to about 2 h, preferably about 30 min forming compound (XLI). The solution is further diluted with an aprotic solvent, preferably DCM, and one equivalent of a primary or secondary amine of formula R5NH2 or (R5)2.NH, where each R5 may be the same or different, as defined above for Formula I, is added. The primary or secondary amine includes a HCl or tosylate salt of an aminoacid ester. Addition of the amine is followed by the addition of a non-nucleophilic base such as a tertiary amine, preferably triethylamine at reduced temperatures of about −78° C. to about 5° C. and preferably at about −78° C. The solution is warmed to ambient temperature and stirred for about 1 h to about 48 h, preferably about 24 h, forming the compound of formula (XLIII). A phosphorus NMR can be acquired to determine the status of the reaction.

The solution is then cooled to about −100° C. to rt, or preferably −78° C. 1 to 10 equivalents, preferably 5 equivalents, of a primary or secondary amine of formula R6NH2 or (R6)2.NH, where each R6 may be the same or different, as defined above for Formula I, is added. The primary or secondary amine includes a HCl or tosylate salt of an aminoacid ester, such as the HCl or tosylate salt of an aminoacid ester. This is followed by the addition of an excess of a non-nucleophilic base such as a tertiary amine, preferably triethylamine (5-10 equivalents) at reduced temperatures of about −78° C. to about 5° C. and preferably at about −78° C. The solution is warmed to ambient temperature and stirred for about 1 h to about 48 h, preferably about 24 h, forming phosphodiamidate (XLIV). The reaction is worked up using standard methods familiar to one skilled in the art, for example extraction with a sodium chloride solution, drying with sodium sulfate and purification by silica gel chromatography.

It is to be understood that changes to this procedure, including solvent switches and optimization of the temperature, familiar to those skilled in the art of organic chemistry, can be made and are anticipated.

Reaction Scheme 13 illustrates an alternative method for synthesizing asymmetrical phosphodiamidates.

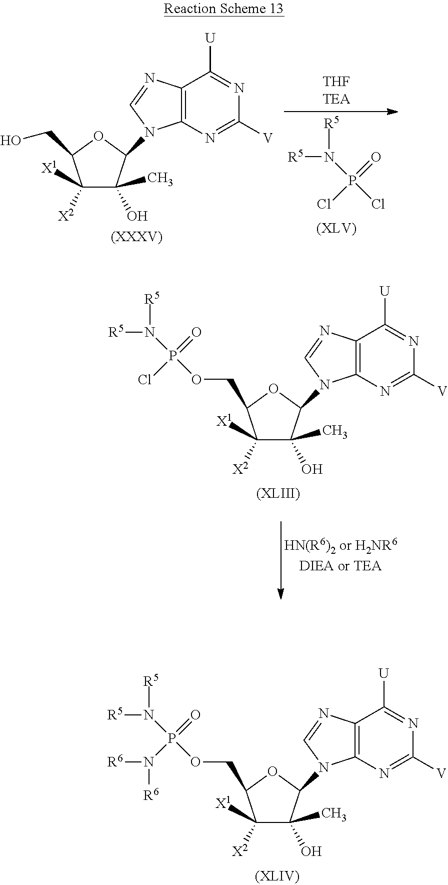

This scheme describes a second general method for synthesizing asymmetrical phosphodiamidates. The nucleoside (XXXV) can be dissolved in a neutral aprotic solvent such as THF or triethyl phosphate or similar solvent, but preferably THF. A non-nucleophilic base such as a tertiary amine or diisopropylethylamine or preferably triethylamine is added in excess, preferably 1.2 equivalents. The solution can be stirred at ambient temperature and 1 to 3 equivalents of an amino acid ester dichloridate (XLV), can be added. Compounds of the general structure (XLV) can be synthesized using techniques familiar to one skilled in the art. A phosphorus NMR can be acquired to determine the status of the formation of the compound of formula (XLIII) The solution is then cooled to −100° C. to rt, or preferably −78° C., and 1 to 10 equivalents, preferably 5 equivalents, of a primary or secondary amine of formula R6NH2 or (R6)2NH are added. The solution is warmed to ambient temperature and stirred for 1 h to 48 h, preferably 24 h, forming phosphodiamidate (XLIV). The reaction is worked up using standard methods familiar to one skilled in the art, for example extraction with a sodium chloride solution, drying with sodium sulfate and purification by silica gel chromatography.

Reaction Scheme 14 illustrates the synthesis of the monophosphate and triphosphate derivatives starting from the compound of formula (XLVI);

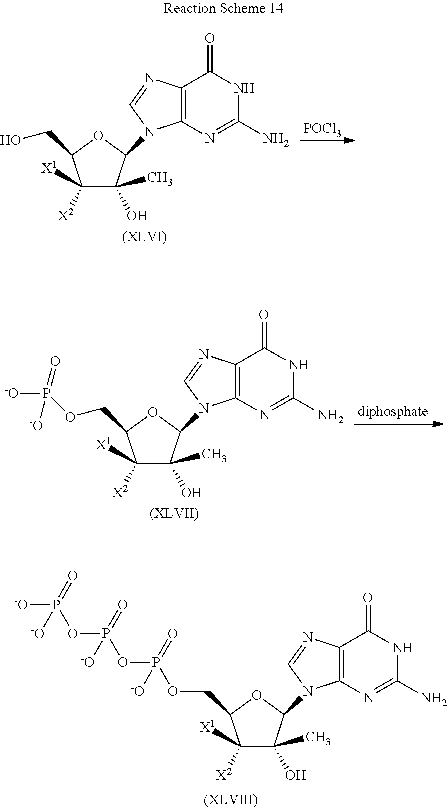

Treatment of ((XLVI)) with POCl3 provides the compound of formula (XLVII) after aqueous workup. Alternatively, treatment with pyrophosphate provides the triphosphate of formula (XLVIII) after aqueous workup. These compounds have additional utility as analytical markers in in vivo studies.

It is to be understood that changes to this procedure, including solvent substitutions and optimization of the temperature, as familiar to those skilled in the art of organic chemistry, can be made and are anticipated.

The general schemes above are preferably carried out in the presence of a suitable solvent. Suitable solvents include hydrocarbon solvents such as benzene and toluene; ether type solvents such as diethyl ether, tetrahydrofuran, diphenyl ether, anisole and dimethoxybenzene; halogenated hydrocarbon solvents such as methylene chloride, chloroform and chlorobenzene; ketone type solvents such as acetone, methyl ethyl ketone and methyl isobutyl ketone; alcohol type solvents such as methanol, ethanol, propanol, isopropanol, n-butyl alcohol and tert-butyl alcohol; nitrile type solvents such as acetonitrile, propionitrile and benzonitrile; ester type solvents such as ethyl acetate and butyl acetate; carbonate type solvents such as ethylene carbonate and propylene carbonate; and the like. These may be used singly or two or more of them may be used in admixture. Preferably an inert solvent is used in the process of the present invention. The term “inert solvent” means a solvent inert under the conditions of the reaction being described in conjunction therewith including, for example, benzene, toluene, acetonitrile, tetrahydrofuran, dimethylformamide, chloroform, methylene chloride (or dichloromethane), diethyl ether, ethyl acetate, acetone, methylethyl ketone, methanol, ethanol, propanol, isopropanol, tert-butanol, dioxane, pyridine, and the like.

### Dosages and Routes of Administration

In general, the compounds of this invention will be administered in a therapeutically effective amount by any of the accepted modes of administration for agents that serve similar utilities. The effective amount will be that amount of the compound of this invention that would be understood by one skilled in the art to provide therapeutic benefits, i.e., the active ingredient, and will thus depend upon numerous factors such as the severity of the disease to be treated, the age and relative health of the subject, the potency of the compound used, the route and form of administration, and other factors. The drug can be administered more than once a day, and in the preferred mode the drug is administered once or twice a day. As indicated above, all of the factors to be considered in determining the effective amount will be well within the skill of the attending clinician or other health care professional.

For example, therapeutically effective amounts of compounds of formula (I) may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-700 mg per day. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915, said patent incorporated herein by reference).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. These patents are incorporated herein by reference.

As indicated above, the compositions in accordance with the invention generally comprise a compound of Formulas I, II or III in combination with at least one pharmaceutically acceptable carrier, excipient or diluent. Some examples of acceptable excipients are those that are non-toxic, will aid administration, and do not adversely affect the therapeutic benefit of the compound of the invention. Such excipient may be any solid, liquid, semi-solid or, in the case of an aerosol composition, gaseous excipient that is generally available to one of skill in the art.

Solid pharmaceutical excipients useful in the invention may include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990). The amount of the compound in a formulation can vary within the full range employed by those skilled in the art. For example, the formulation will contain, on a weight percent (wt %) basis, from about 0.01-99.99 wt % wherein the compound is a compound of Formula I based on the total formulation, with the balance being one or more suitable pharmaceutical excipients. Preferably, the compound is present at a level of about 1-80 wt %. Pharmaceutical formulations containing a compound in accordance with the invention are described further below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics and Therapeutic Potential,” Ann. Rep. Med. Chem., 35:201-210 (2000).

Even further, the present invention is directed to a pharmaceutical composition comprising a therapeutically effective amount of a compound of the present invention in combination with a therapeutically effective amount of another agent active against hepatitis C virus. Such agents include those that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety. Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharmaceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-5703 10 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of Formula I, II or III and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention utilize a combination of a compound of Formula I, II or III and a compound having anti-HCV activity such as those selected from the group consisting of interleukin 2, interleukin 6, interleukin 12, a compound that enhances the development of a type 1 helper T cell response, interfering RNA, anti-sense RNA, Imiqimod, ribavirin, an inosine 5′ monophosphate dehydrogenase inhibitor, amantadine, and rimantadine.

### Anti-Hepatitis C Activity Assays

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. J. of Vir., 73:1649-1654, 1999; Ishii et al., Hepatology, 29:1227-1235, 1999; Lohmann et al., J. Bio. Chem., 274:10807-10815, 1999; and Yamashita et al., J. of Bio. Chem., 273:15479-15486, 1998.

WO 97/12033 relates to HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1 (Supp 4) 18-24.

Screens that measure reductions in kinase activity from HCV drugs were disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs were disclosed in U.S. Pat. No. 5,861,267 to Su et al., U.S. Pat. No. 5,739,002 to De Francesco et al., and U.S. Pat. No. 5,597,691 to Houghton et al.

### EXAMPLES

Embodiments of the present invention will now be described by way of example only with respect to the following non-limiting examples.

### General Procedures

All experiments involving water-sensitive compounds were conducted under scrupulously dry conditions. Anhydrous tetrahydrofuran (THF) and dichloromethane were purchased from Aldrich and used directly. The purine derivative 2-amino-6-chloropurine or equivalently, 6-chloro-9H-purin-2-amine, was purchased from Aldrich. Salts of amino acid esters were prepared as described in PCT Int. Appl. (2010), WO 2010081082 A2 20100715. Chloro phosphoramidates used to make nucleoside phosphoramidates were prepared as described in PCT Int. Appl. (2010), WO 2010081082 A2, and in Bioorganic & Medicinal Chemistry Letters (2011), 21(19), 6007-6012. Column chromatography refers to flash column chromatography carried out using Merck silica gel 60 (40-60 μm) as stationary phase. Proton, carbon, and phosphorus nuclear magnetic resonance (1H, 13C, 31P NMR) spectra were recorded on Bruker Avance spectrometers operating either at 500, 125, and 202 MHz or at 300, 75, and 121 MHz or a Varian Unity Inova instrument operating at 400, 100, and 161.9 MHz. The solvents used are indicated for each compound. All 13C and 31P spectra were recorded proton decoupled. Chemical shifts for 1H and 13C spectra are in parts per million downfield from tetramethylsilane. Coupling constants are referred to as J values. Signal splitting patterns are described as singlet (s), doublet (d), triplet (t), quartet (q), broad signal (br), doublet of doublet (dd), doublet of triplet (dt), or multiplet (m). Chemical shifts for 31P spectra are in parts per million relative to an external phosphoric acid standard. Some of the proton and carbon NMR signals were split because of the presence of (phosphate) diastereoisomers in the samples. The mode of ionization for mass spectrometry was fast atom bombardment (FAB) using MNOBA (m-nitrobenzyl alcohol) as matrix for some compounds. Electrospray mass spectra were obtained using a Waters LCT time-of-flight mass spectrometer coupled to a Waters M600 HPLC pump. Samples were dissolved in methanol and injected into the solvent stream via a Rheodyne injector. The mobile phase used was methanol at a flow rate of 200 μL/min. The electrospray source was operated at a temperature of 130° C. with a desolvation temperature of 300° C., a capillary voltage of 3 kV, and cone voltage of 30 V. Data were collected in the continuum mode over the mass range 100-2000 amu and processed using Masslynx 4.1 software. Accurate mass measurements were facilitated by the introduction of a single lockmass compound of known elemental composition into the source concurrently with sample.

### Example 1

### ((2R,3R,4S,5R)-5-(2-Amino-6-chloro-9H-purin-9-yl)-4-(benzoyloxy)-3-fluoro-4-methyltetrahydrofuran-2-yl)methyl benzoate

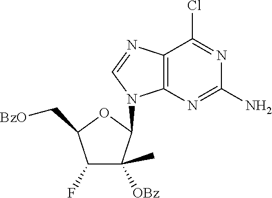

Compound of Example 1 was prepared in a multi-step synthesis starting from readily available ((2R,3R,4R,5S)-3,4-dihydroxy-5-methoxytetrahydrofuran-2-yl)methyl benzoate (I). Compound I was prepared in a two step sequence from commercially available 5-(hydroxymethyl)-2,2-dimethyltetrahydrofuro[3,2-d][1,3]dioxol-6-ol, (1,2-isopropylidene D-xylofuranoside), involving benzoylation with benzoyl chloride in DCM with TEA, followed by conversion to a mixture of anomeric methyl furanosides in methanol with Iodine. The mixture was separated by column chromatography (hexanes/ethyl acetate, gradient) to provide the beta methyl furanoside, ((2R,3R,4R,5R)-3,4-dihydroxy-5-methoxytetrahydrofuran-2-yl)methyl benzoate, and the alpha methyl furanoside I which was used in Step 1 below.

### Step 1 Preparation of ((3S,4S,5S)-3-fluoro-4-hydroxy-5-methoxytetrahydrofuran-2-yl)methyl benzoate

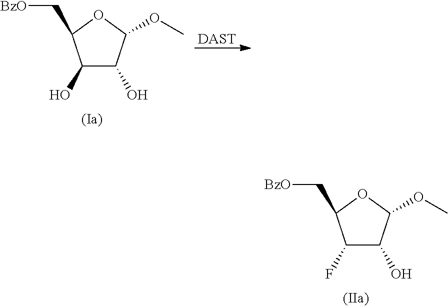

To a solution of ((3R,4R,5S)-3,4-dihydroxy-5-methoxytetrahydrofuran-2-yl)methyl benzoate Ia (7.81 gm, 0.028 mol) (mixture of both α and β anomers) in anhydrous CH2Cl2 was added DAST drop wise at 0° C. and stirred for 4 hrs at 0° C. The reaction mixture was washed with saturated aqueous NaHCO3 and extracted with CH2Cl2. The organic layers were dried with anhydrous Na2SO4, filtered and concentrated. The resulting residue was chromatographed on a column of silica gel using a stepwise gradient of ethyl acetate (5-20%) in hexane to obtained 1.01 gm of compound IIa and 1.04 gm of a mixture of compounds.

1H NMR (200 MHz, CDCl3): δ 8.0 (m, 2H), 7.6 (m, 1H), 7.5 (m, 2H), 5.1 (d, J=4 Hz, ½H), 4.97 (d, J=4.8 Hz, 1H), 4.8 (d, J=4 Hz, ½H), 4.65 (m, 1H), 4.5 (m, 2H), 4.4 (m, 1H), 3.5 (s, 3H).

19F NMR (188 Hz, CDCl3): δ −195.53, −195.66, −195.79, −195.83, −195.96, −196.08

### Step 2 Preparation of ((2R,3R,5S)-3-fluoro-5-methoxy-4-oxotetrahydrofuran-2-yl)methyl benzoate

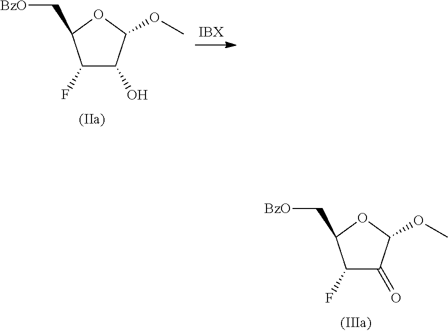

To a solution of ((3S,4S,5S)-3-fluoro-4-hydroxy-5-methoxytetrahydrofuran-2-yl)methyl benzoate IIa (1.01 gm, 3.74 mmol) in DMSO, was added 5.23 gm of 2-Iodoxybenzoic acid (IBX). The suspension becomes homogeneous after 30 minutes and was stirred at room temperature for 15 hrs. The reaction mixture was diluted in ethyl acetate and washed with saturated aqueous NaHCO3 and water, dried (anhyd.Na2SO4) and concentrated. A silica gel column chromatographic purification of this residue using a stepwise gradient of ethyl acetate (20-40%) in hexane afforded 910 mg of ((2R,3R,5S)-3-fluoro-5-methoxy-4-oxotetrahydrofuran-2-yl)methyl benzoate IIIa. Compound IIIa exists as a mixture of keto and hydrated keto forms.

1H NMR (200 MHz, CDCl3): δ 8.00 (m, 2H), 7.5 (m, 1H), 7.4 (m, 2H), 4.8-5.2 (m, 1H), 4.5 (m, 1H), 4.45 (m, 2H), 3.43 (s, 3H).

19F NMR (188 Hz, CDCl3): δ −186.29, 186.44, −186.58, −186.65.

### Step 3 Preparation of ((2R,3R,4S,5S)-3-fluoro-4-hydroxy-5-methoxy-4-methyltetrahydrofuran-2-yl)methyl benzoate

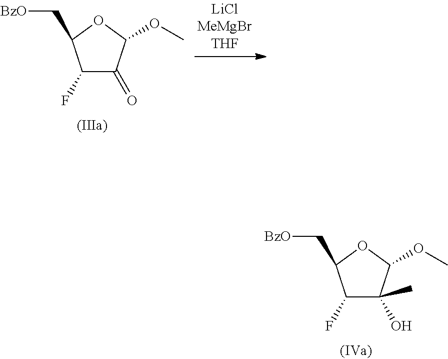

To a solution of compound IIIa (910 mg, 3.39 mmol) in 20 mL anhydrous THF, anhydrous LiCl (431 mg, 10.18 mmol) was added and stirred at 40° C. for 1 h. Later this solution was cooled to −40° C. and MeMgBr (2.26 mL, 6.79 mmol) was added drop wise and stirred for 3 h at −40° C. The resulting solution was quenched with 1N HCl, diluted with ethyl acetate and washed with sat.aq.NaHCO3. The organic layers were dried (anhyd.Na2SO4), filtered, concentrated and chromatographed on a silica gel using stepwise gradient of ethyl acetate (5-20%) in hexane to afford 550 mg of compound IVa and 312 mg of recovered starting material compound IIIa.

1H NMR (200 MHz, CDCl3): δ 8.05 (m, 2H), 7.61 (m, 1H), 7.45 (m, 2H), 4.6 (m, ½H), 4.55 (m, 2H), 4.52 (s, 1H), 4.4 (m, 1H), 4.28 (d, J=2.8 Hz, ½H), 3.47 (s, 3H), 1.38 (d, J=4 Hz, 3H).

19F NMR (188 Hz, CDCl3): δ −184.72, −184.86, −185.02, −185.16

### Step 4 Preparation of ((2R,3R,4S,5S)-4-(benzoyloxy)-3-fluoro-5-methoxy-4-methyltetrahydrofuran-2-yl)methyl benzoate

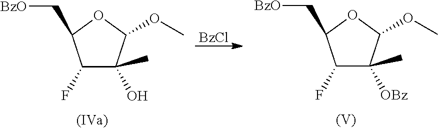

A solution of ((2R,3R,4S,5S)-3-fluoro-4-hydroxy-5-methoxy-4-methyltetrahydrofuran-2-yl)methyl benzoate IVa (550 mg, 1.93 mmol) in 15 mL of pyridine was treated with benzoyl chloride (674 μL, 5.8 mmol) and DMAP (472 mg, 3.9 mmol), and then heated to 75° C. for 15 hrs. The reaction mixture was concentrated in vacuo, diluted with ethyl acetate, washed with 1N HCl and saturated aqueous NaHCO3, dried (anhyd. Na2SO4), filtered and concentrated. Column chromatographic purification using a stepwise gradient of ethyl acetate (2-10%) in hexane afforded 718 mg of compound V.

1H NMR (200 MHz, CDCl3): δ 8.05 (m, 4H), 7.6 (m, 2H), 7.4 (m, 4H), 5.2 (s, 1H), 5.07 (d, J=4 Hz, ½H), 4.8 (d, J=4 Hz, ½H), 4.6 (m, 2H), 4.5 (m, 1H), 3.47 (s, 3H), 1.4 (d, J=2.5 Hz, 3H).

19F NMR (188 Hz, CDCl3): δ 183.29, −183.45, −183.58, −183.74.

### Step 5 Preparation of ((2R,3R,4S)-5-acetoxy-4-(benzoyloxy)-3-fluoro-4-methyltetrahydrofuran-2-yl)methyl benzoate

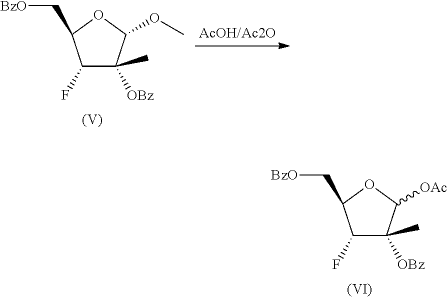

To a cooled (ice-bath) solution of ((2R,3R,4S,5S)-4-(benzoyloxy)-3-fluoro-5-methoxy-4-methyltetrahydrofuran-2-yl)methyl benzoate (V) (540 mg) in 3 mL acetic acid was added 1.5 mL acetic anhydride followed by drop wise addition of H2SO4 (10 drops) and gradually the reaction mixture was warmed to room temperature and stirred for 12 hrs. The reaction mixture was diluted with ethyl acetate and was poured into sat.aq.NaHCO3, and stirred for 2 hrs. The organic layer was washed with water and brine, dried over anhydrous Na2SO4, filtered and concentrated. Chromatography of the residue on a column of silica gel, using step wise gradient of ethyl acetate (5-15%) in hexane gave 420 mg of mixture of anomeric acetates (VI).

1H NMR (200 MHz, CDCl3): δ 8.05 (m, 4H), 7.6 (m, 2H), 7.4 (m, 4H), 6.72 (d, J=2 Hz), 6.43 (s, 1H), 5.22-5.32 (m, 1H), 4.96-5.04 (m, 1H), 4.8 (m, 1H), 4.4-4.7 (m, 2H), 2.11 (s, 3H), 2.01 (s, 3H), 1.82 (s, 3H), 1.74 (d, J=2.2 Hz, 3H).

19F NMR (188 Hz, CDCl3): δ 182.27, −182.42, −182.55, −182.69, −213.90, −213.99, −214.17, −214.26

### Step 6 Preparation of ((2R,3R,4S,5R)-5-(2-amino-6-chloro-9H-purin-9-yl)-4-(benzoyloxy)-3-fluoro-4-methyltetrahydrofuran-2-yl)methyl benzoate

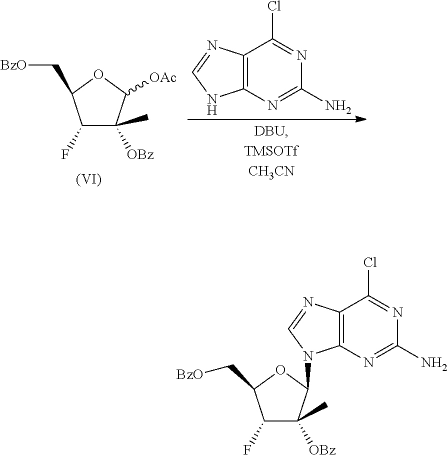

A solution of ((2R,3R,4S)-5-acetoxy-4-(benzoyloxy)-3-fluoro-4-methyltetrahydrofuran-2-yl)methyl benzoate (VI), as a mixture of anomeric acetates (91 mg, 0.22 mmol) in 5 mL acetonitrile was prepared. 6-Chloro-2-aminopurine (445 mg, 0.26 mmol) and DBU (99 μL, 0.65 mmol) were added and the solution was cooled to 0° C. TMSOTf (167 μL, 0.92 mmol) was added drop wise, and the resulting mixture was heated to 65° C. for 12 hrs. The reaction mixture was diluted with CH2Cl2 and washed with sat.aq.NaHCO3 and brine. A column chromatographic purification on silica gel using a step wise gradient of ethyl acetate (40-65%) in hexane gave 68 mg (0.13 mmol, 58%) of protected nucleoside of ((2R,3R,4S,5R)-5-(2-amino-6-chloro-9H-purin-9-yl)-4-(benzoyloxy)-3-fluoro-4-methyltetrahydrofuran-2-yl)methyl benzoate.

1H NMR (200 MHz, CDCl3): δ 8.01 (m, 4H), 7.99 (s, 1H), 7.8 (m, 4H), 7.45 (m, 6H), 6.52 (d, J=1.6 Hz, 1H), 5.8 (d, J=6 Hz, ½H), 5.55 (d, J=4 Hz, ½H), 5.44 (bs, 2H), 4.9 (m, 1H), 4.6-4.8 (m, 2H), 1.52 (d, J=1.2 Hz, 3H).

19F NMR (188 Hz, CDCl3): δ −194.19 (2), −194.33 (2)

### Example 2

### (2R,3S,4R,5R)-2-(2-amino-6-methoxy-9H-purin-9-yl)-4-fluoro-5-(hydroxymethyl)-3-methyltetrahydrofuran-3-ol

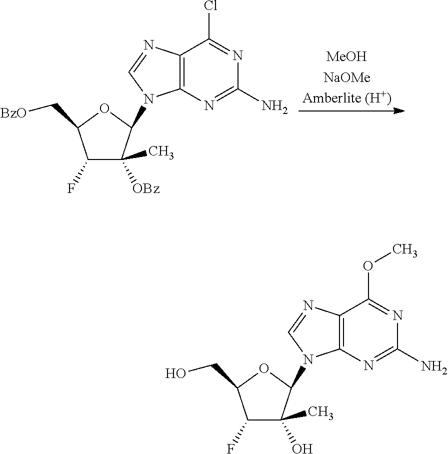

((2R,3R,4S,5R)-5-(2-Amino-6-chloro-9H-purin-9-yl)-4-(benzoyloxy)-3-fluoro-4-methyltetrahydrofuran-2-yl)methyl benzoate 68 mg (0.13 mmol), was dissolved in 3 mL of MeOH and 1 mL of NaOMe (in MeOH) was added. The solution was stirred at room temp for 12 hrs. This mixture was neutralized with acidic resin (Amberlite, H+), filtered, concentrated and purified by silica gel column chromatography using a stepwise gradient of MeOH (2-5%) in CH2Cl2 to give 36 mg (0.114 mmol, 88%) of (2R,3S,4R,5R)-2-(2-amino-6-methoxy-9H-purin-9-yl)-4-fluoro-5-(hydroxymethyl)-3-methyltetrahydrofuran-3-ol.

1H NMR (200 MHz, CDCl3): δ 8.17 (s, 1H), 5.97 (d, J=3 Hz, 1H), 5.3 (d, J=8 Hz, ½H), 5.01 (d, J=8 Hz, ½H), 4.3 (m, 1H), 4.04 (s, 3H), 3.8-4.0 (m, 2H), 1.05 (s, 3H).

19F NMR (188 Hz, CDCl3): δ −216.71, −216.79, −216.99, −217.07

### Example 3

### 2-amino-9-((2R,3S,4R,5R)-4-fluoro-3-hydroxy-5-(hydroxymethyl)-3-methyltetrahydrofuran-2-yl)-1H-purin-6(9H)-one

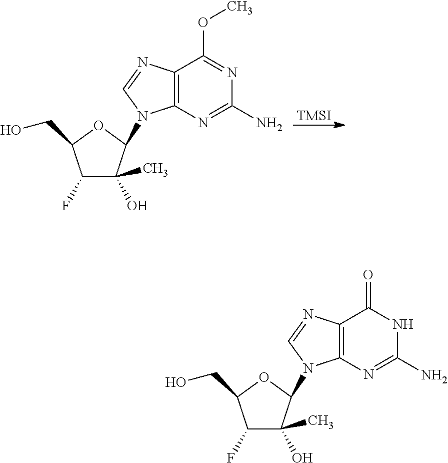 [SMILES: "TMSI" ; COc1nc(N)nc2c1ncn2[C@@H]1O[C@H](CO)[C@@H](F)[C@@]1(C)O ; C[C@@]1(O)[C@H](F)[C@@H](CO)O[C@H]1n1cnc2c(=O)[nH]c(N)nc21]

To (2R,3S,4R,5R)-2-(2-amino-6-methoxy-9H-purin-9-yl)-4-fluoro-5-(hydroxymethyl)-3-methyltetrahydrofuran-3-ol (30 mg, 0.1 mmol) in acetonitrile (3.0 mL) under nitrogen was added Hunig's base (52 □L, 0.3 mmol), followed by the addition of NaI (99 mg, 0.5 mmol) and TMSCl (64 □L, 0.5 mmol). The contents were stirred under nitrogen for 16 h. After completion of the reaction (monitored by TLC), triethyl amine (30 □L, 0.3 mmol) was added. The reaction was then concentrated under vacuum. The solids were then dissolved in methanol/water mixture (1.5 mL/0.5 mL) and stirred for 15 minutes. The crude mixture was then loaded on a silica gel column. Elution with CHCl3/MeOH (0-20%) afforded the desired product 2-amino-9-((2R,3S,4R,5R)-4-fluoro-3-hydroxy-5-(hydroxymethyl)-3-methyltetrahydrofuran-2-yl)-1H-purin-6(9H)-one, (19 mg, 0.063 mmol, 63%).

### Example 4

### (2S,3R,4R,5R)-2-(2-Amino-6-methoxy-9H-purin-9-yl)-4-fluoro-5-(hydroxymethyl)-3-methyltetrahydrofuran-3-ol

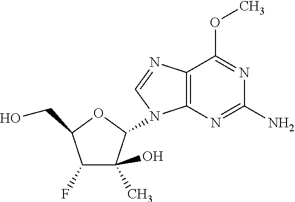 [SMILES: COc1nc(N)nc2c1ncn2[C@H]1O[C@H](CO)[C@@H](F)[C@]1(C)O]

The compound of Example 4 could be synthesized in multiple steps starting from ((2R,3R,4R,5R)-3,4-dihydroxy-5-methoxytetrahydrofuran-2-yl)methyl benzoate which was prepared as described in Example 1.

### Step 1 Preparation of ((2R,3S,4S,5R)-3-fluoro-4-hydroxy-5-methoxytetrahydrofuran-2-yl)methyl benzoate

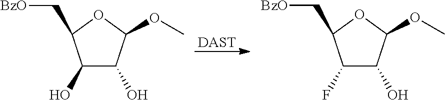

The beta methyl furanoside, ((2R,3R,4R,5R)-3,4-dihydroxy-5-methoxytetrahydrofuran-2-yl)methyl benzoate (1.5 g, 5.6 mmol) was dissolved in dry DCM (50 mL) and treated with DAST (4.4 mL, 33.58 mmol) and stirred at RT. Stirring was continued overnight, cooled to 0° C. and poured on cold saturated sodium bicarbonate solution (100 mL) slowly for 15 min. The layers were separated and extracted with DCM (2×75 mL). The combined organic layers were dried (sodium sulfate) and concentrated. The crude product was purified by column chromatography using gradient mixtures of hexane/ethyl acetate (8:2) to give 710 mg of ((2R,3S,4S,5R)-3-fluoro-4-hydroxy-5-methoxytetrahydrofuran-2-yl)methyl benzoate as white solid.

### Step 2 Preparation of ((2R,3R,5R)-3-fluoro-5-methoxy-4-oxotetrahydrofuran-2-yl)methyl benzoate

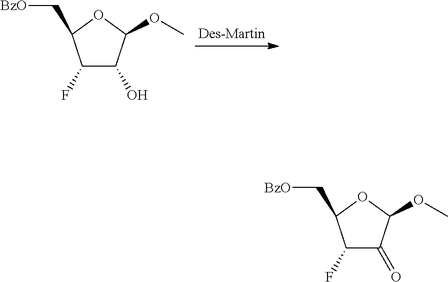

((2R,3S,4S,5R)-3-Fluoro-4-hydroxy-5-methoxytetrahydrofuran-2-yl)methyl benzoate (710 mg, 2.63 mmol) was dissolved in dichloromethane (50 mL) and treated with Dess-Martin periodinane (1.67 mg, 3.94 mmol) at 0° C. The reaction mixture was stirred overnight at RT, then evaporated to dryness, and anhydrous ether (45 mL) was added. The solution was stirred under nitrogen for 30 minutes, and the solids were filtered through anhydrous MgSO4. The filtrate was shaken with saturated sodium bicarbonate (50 mL, with 6.2 g of sodiumthiosulfate) until a clear solution was obtained. The organic layer was washed with brine solution (50 mL), dried with MgSO4, filtered, and concentrated to dryness. The crude ((2R,3R,5R)-3-fluoro-5-methoxy-4-oxotetrahydrofuran-2-yl)methyl benzoate was dissolved in anhydrous DCM (50 mL) and stirred with anhydrous MgSO4 under nitrogen for 18 h. The solution was filtered and concentrated to give 620 mg (2.3 mmol, 88%) of oxidized product as gummy material in 6:4 ratio of ketone to hydrate.

### Step 3 Preparation of ((2R,3R,4R,5R)-3-fluoro-4-hydroxy-5-methoxy-4-methyltetrahydrofuran-2-yl)methyl benzoate

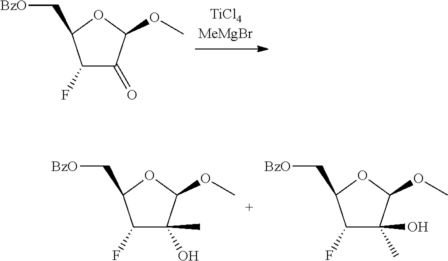

TiCl4 (1.0 mL, 9.07 mmol) was added slowly to anhydrous ether (60 mL) at −78° C. for 30 min. To the resulting yellow solution was added 3M ether solution of methyl magnesium bromide (3.02 mL, 9.07 mmol) at −78° C. for 30 min. The reaction mixture changed from yellow to dark orange, and was warmed to −30° C. and added to a solution of the oxidized sugar, ((2R,3R,5R)-3-fluoro-5-methoxy-4-oxotetrahydrofuran-2-yl)methyl benzoate (610 mg, 2.3 mmol), dissolved in 20 mL of anhydrous diethyl ether. This solution was stirred for 5 hrs at −30° C. and, poured over cold water (100 mL) and extracted twice with 50 mL ether. The organics were dried over Na2SO4, concentrated and purified by column chromatography using hexanes/ethyl acetate (82:18) to give 170 mg (0.6 mmol, 26%) of ((2R,3R,4S,5R)-3-fluoro-4-hydroxy-5-methoxy-4-methyltetrahydrofuran-2-yl)methyl benzoate and ((2R,3R,4R,5R)-3-fluoro-4-hydroxy-5-methoxy-4-methyltetrahydrofuran-2-yl)methyl benzoate as an inseparable gummy solid.

### Step 4 Preparation of ((2R,3R,4R,5R)-4-(benzoyloxy)-3-fluoro-5-methoxy-4-methyltetrahydrofuran-2-yl)methyl benzoate

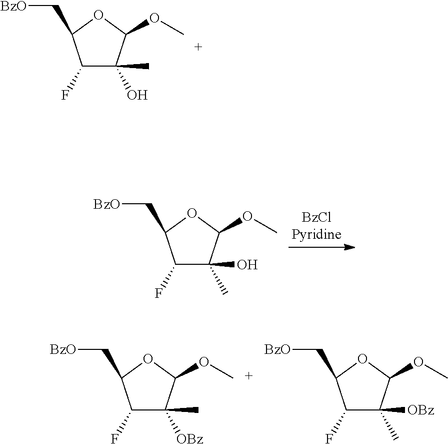

The mixture from Step 3 (170 mg, 0.6 mmol) was dissolved in anhydrous pyridine (6 mL) and benzoyl chloride (0.09 mL, 078 mmol) was added at RT. The solution was stirred at 65° C. overnight. The solvent was evaporated and the residue was taken up in water and extracted with eiethyl ether (3×20 mL). The organics were dried (Na2SO4), concentrated, and purified by column chromatography using hexanes/ethyl acetate (95:5 to 92:8) to give 140 mg (0.36 mmol, 60%) of ((2R,3R,4R,5R)-4-(benzoyloxy)-3-fluoro-5-methoxy-4-methyltetrahydrofuran-2-yl)methyl benzoate (first eluted spot, arabinose) and 50 mg (0.13 mmol, 20%) of ((2R,3R,4S,5R)-4-(benzoyloxy)-3-fluoro-5-methoxy-4-methyltetrahydrofuran-2-yl)methyl benzoate (second eluted spot, ribose) as thick oils.

### Step 5 Preparation of ((2R,3R,4R)-5-acetoxy-4-(benzoyloxy)-3-fluoro-4-methyltetrahydrofuran-2-yl)methyl benzoate

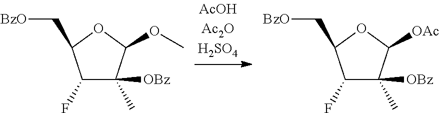

((2R,3R,4R,5R)-4-(Benzoyloxy)-3-fluoro-5-methoxy-4-methyltetrahydrofuran-2-yl)methyl benzoate (110 mg, 0.28 mmol) was dissolved in acetic acid (3 mL) and added acetic anhydride (0.21 mL, 2.3 mmol) followed by sulfuric acid (3 drops). The resulting mixture was stirred overnight at RT, poured on cold water (50 mL) and extracted with ethyl acetate (2×25 mL). The combined organic layers were washed with 25 mL saturated sodium bicarbonate solution. Dried on Na2SO4, concentrated, purified by column chromatography using hexanes/ethyl acetate (9:1) to give 88 mg (0.21 mmol, 75%) of ((2R,3R,4R)-5-acetoxy-4-(benzoyloxy)-3-fluoro-4-methyltetrahydrofuran-2-yl)methyl benzoate as thick oil.

### Step 6 Preparation of ((2R,3R,4R,5S)-5-(2-amino-6-chloro-9H-purin-9-yl)-4-(benzoyloxy)-3-fluoro-4-methyltetrahydrofuran-2-yl)methyl benzoate

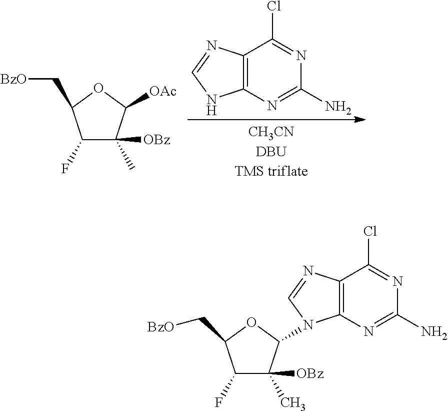

To a stirred solution of the sugar derivative ((2R,3R,4R)-5-acetoxy-4-(benzoyloxy)-3-fluoro-4-methyltetrahydrofuran-2-yl)methyl benzoate (190 mg, 0.457 mmol) in anhydrous acetonitrile (25 mL) was added 2-amino-6-chloro purine (85 mg, 0.50 mmol), DBU (0.20 mL, 1.4 mmol) and followed by TMSOTf (0.33 mL, 1.8 mmol) at 0° C. The solution was warmed to reflux, and stirred overnight (18 h). The solution was cooled to RT, treated with saturated sodium bicarbonate (50 mL), and extracted with DCM (2×50 mL). The organics were dried with Na2SO4, concentrated, and purified by column chromatography using DCM/MeOH (97:3) to give 180 mg (0.34 mmol, 75%) of benzoylated nucleoside ((2R,3R,4R,5S)-5-(2-amino-6-chloro-9H-purin-9-yl)-4-(benzoyloxy)-3-fluoro-4-methyltetrahydrofuran-2-yl)methyl benzoate as off white solid.

### Step 7 Preparation of (2S,3R,4R,5R)-2-(2-amino-6-methoxy-9H-purin-9-yl)-4-fluoro-5-(hydroxymethyl)-3-methyltetrahydrofuran-3-ol

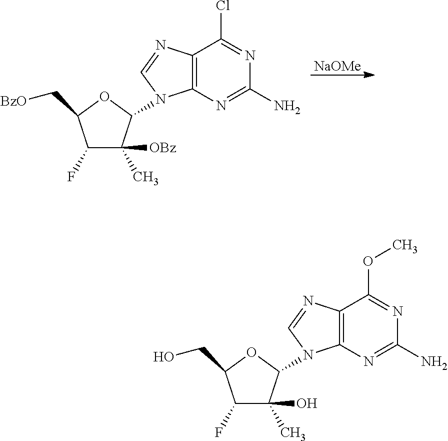

The protected nucleoside ((2R,3R,4R,5S)-5-(2-amino-6-chloro-9H-purin-9-yl)-4-(benzoyloxy)-3-fluoro-4-methyltetrahydrofuran-2-yl)methyl benzoate (98 mg, 0.18 mmol) was debenzoylated with sodium methoxide (5 eq) in methanol (5 mL) by stirring overnight at RT. The solvent was evaporated and the crude was purified by column chromatography, using DCM/MeOH (9:1) and then with ethyl acetate/methanol (99:1) to give 21 mg (0.07 mmol, 37%) of nucleoside (2S,3R,4R,5R)-2-(2-amino-6-methoxy-9H-purin-9-yl)-4-fluoro-5-(hydroxymethyl)-3-methyltetrahydrofuran-3-ol as an off white solid.

### Example 5

### (2S,3R,4R,5R)-2-(2-amino-6-chloro-9H-purin-9-yl)-4-fluoro-5-(hydroxymethyl)-3-methyltetrahydrofuran-3-ol

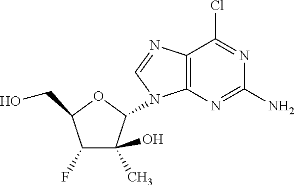 [SMILES: C[C@]1(O)[C@H](F)[C@@H](CO)O[C@@H]1n1cnc2c(Cl)nc(N)nc21]

The process of Example 4, Step 7, also provide the nucleoside of Example 5. Thus, protected nucleoside ((2R,3R,4R,5S)-5-(2-amino-6-chloro-9H-purin-9-yl)-4-(benzoyloxy)-3-fluoro-4-methyltetrahydrofuran-2-yl)methyl benzoate (98 mg, 0.18 mmol) was debenzoylated with sodium methoxide (5 eq) in methanol (5 mL) by stirring overnight at RT. The solvent was evaporated and the crude was purified by column chromatography, using DCM/MeOH (9:1) and then with ethyl acetate/methanol (99:1) to give 5 mg (0.016 mmol, 9%) of nucleoside (2S,3R,4R,5R)-2-(2-amino-6-chloro-9H-purin-9-yl)-4-fluoro-5-(hydroxymethyl)-3-methyltetrahydrofuran-3-ol.

### Example 6

### (2R,3S,4R,5R)-2-(2-amino-6-ethoxy-9H-purin-9-yl)-4-fluoro-5-(hydroxymethyl)-3-methyltetrahydrofuran-3-ol

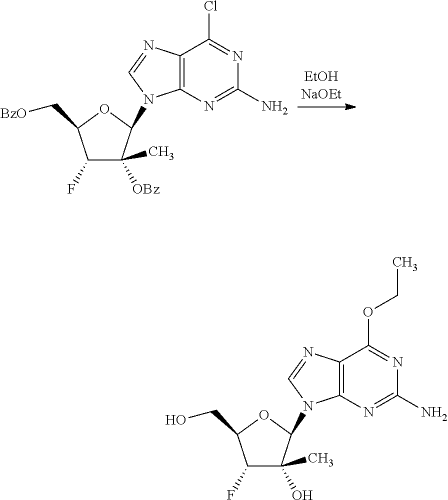

Cleavage of the benzoates and conversion of 6-Chloro to 6-Ethoxy was achieved in one step. ((2R,3R,4S,5R)-5-(2-Amino-6-chloro-9H-purin-9-yl)-4-(benzoyloxy)-3-fluoro-4-methyltetrahydrofuran-2-yl)methyl benzoate (610 mg, 1.16 mmol) was dissolved in 10 mL of EtOH followed by addition of freshly prepared NaOEt (9.2 mmol) in ethanol. This reaction mixture was stirred at room temp for 24 hrs. Neutralized with acidic resin and purified by column chromatography to give 247 mg (0.75 mmol, 65%) of (2R,3S,4R,5R)-2-(2-amino-6-ethoxy-9H-purin-9-yl)-4-fluoro-5-(hydroxymethyl)-3-methyltetrahydrofuran-3-ol.

1H NMR (200 MHz, CD3OD): δ 8.17 (s, 1H), 5.98 (d, J=0.8 Hz, 1H), 5.31 (d, J=7.6 Hz, 0.5H), 5.04 (d, J=7.6 Hz, 0.5H), 4.52 (q, J=7 Hz, 2H), 4.3 (m, 1H), 3.92 (dq, J=16.6 Hz, J=2.2 Hz, 2H), 1.42 (t, J=7 Hz, 3H), 1.06 (s, 3H)

19F NMR (188 MHz, CD3OD): δ −216.915, −216.993, −217.200, −217.278

### Example 7

### (2R,3S,4R,5R)-2-(2-Amino-6-(azetidin-1-yl)-9H-purin-9-yl)-4-fluoro-5-(hydroxymethyl)-3-methyltetrahydrofuran-3-ol

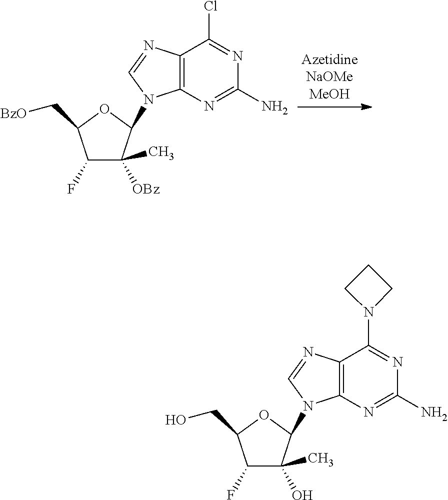

((2R,3R,4S,5R)-5-(2-Amino-6-chloro-9H-purin-9-yl)-4-(benzoyloxy)-3-fluoro-4-methyltetrahydrofuran-2-yl)methyl benzoate (542 mg, 1.03 mmol) was dissolved in 10 mL of EtOH followed by addition of azetidine (0.42 mL, 6.2 mmol) and Et3N (1.4 mL, 10.3 mmol) and heated to 50° C. for 18 hrs. The volatiles were removed under reduced pressure and the resulting pale brown residue was dissolved in MeOH followed by addition of NaOMe (6.2 mmol) and stirred at room temperature for 15 hrs. After neutralized with acidic resin (Amberlite) and purification by column chromatography (DCM/MeOH), 239 mg (0.71 mmol, 68%) of (2R,3S,4R,5R)-2-(2-Amino-6-(azetidin-1-yl)-9H-purin-9-yl)-4-fluoro-5-(hydroxymethyl)-3-methyltetrahydrofuran-3-ol was obtained.

1H NMR (200 MHz, CD3OD): δ 8.02 (s, 1H), 5.90 (d, J=0.6 Hz, 1H), 5.28 (d, J=10 Hz, 0.5H), 5.03 (d, J=10 Hz, 0.5H), 4.42 (m, 5H), 3.94 (dq, J=16 Hz, J=2 Hz, 2H), 2.43 (p, J=8 Hz, 2H), 1.01 (s, 3H)

19F NMR (188 MHz, CD3OD): δ −216.779, −216.870, −216.857, −217.064, −217.142

### Example 8

### (2R,3S,4R,5R)-2-(6-Amino-9H-purin-9-yl)-4-fluoro-5-(hydroxymethyl)-3-methyltetrahydrofuran-3-ol

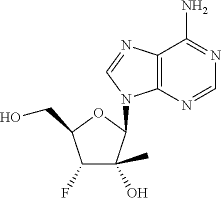 [SMILES: C[C@@]1(O)[C@H](F)[C@@H](CO)O[C@H]1n1cnc2c(N)ncnc21]

The compound of Example 8 could be prepared in two steps starting from 6-chloropurine and the sugar derivative, (2R,3S,4R,5R)-2-(6-amino-9H-purin-9-yl)-4-fluoro-5-(hydroxymethyl)-3-methyltetrahydrofuran-3-ol VI, prepared in Example 1.

### Step 1 Preparation of ((2R,3R,4S,5R)-4-(benzoyloxy)-5-(6-chloro-9H-purin-9-yl)-3-fluoro-4-methyltetrahydrofuran-2-yl)methyl benzoate

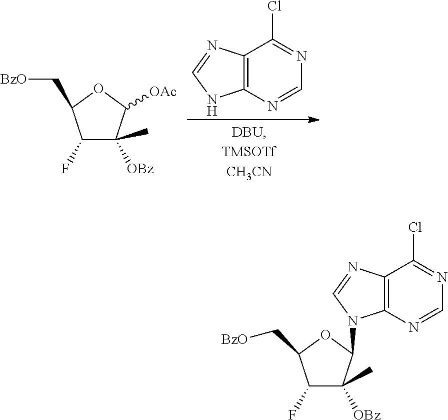

To a suspension of 6-chloropurine (294 mg, 1.90 mmol) and compound VI (660 mg, 1.59 mmol) in anhydrous CH3CN (15 mL) was added DBU (710 μL, 4.76 mmol). The resulting clear solution was cooled to 0° C., and TMS triflate (1.2 mL, 6.7 mmol) was added dropwise. The solution was then heated to 70° C. for 12 hrs. The reaction mixture was diluted with EtOAc and washed with saturated aqueous NaHCO3 and brine. The organic layers were dried with anhydrous Na2SO4, filtered, and concentrated. The resulting residue purified by column chromatography using a stepwise gradient of ethyl acetate (20-40%) in hexane to obtain 490 mg (0.96 mmol, 60%) of ((2R,3R,4S,5R)-4-(benzoyloxy)-5-(6-chloro-9H-purin-9-yl)-3-fluoro-4-methyltetrahydrofuran-2-yl)methyl benzoate.

1H NMR (200 MHz, CDCl3): δ 8.76 (s, 1H), 8.37 (s, 1H), 8.1 (m, 4H), 7.66-7.4 (m, 6H), 6.68 (d, J=3.6 Hz, 1H), 5.8 (m, 0.5H), 5.55 (d, J=4 Hz, 0.5H), 4.8 (bs, 2H), 4.7 (m, 1H), 1.6 (s, 3H)

19F NMR (188 MHz, CDCl3): δ −194.92(2), −195.11(2)

### Step 2 Preparation of (2R,3S,4R,5R)-2-(6-Amino-9H-purin-9-yl)-4-fluoro-5-(hydroxymethyl)-3-methyltetrahydrofuran-3-ol

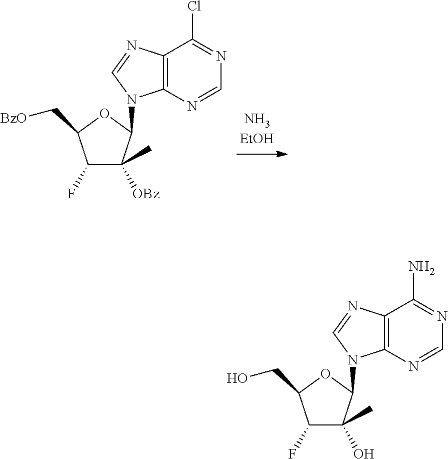

The protected nucleoside ((2R,3R,4S,5R)-4-(benzoyloxy)-5-(6-chloro-9H-purin-9-yl)-3-fluoro-4-methyltetrahydrofuran-2-yl)methyl benzoate (491 mg, 0.96 mmol) was taken in ethanol (20 mL), and a saturated NH3/EtOH solution (20 mL) was added and this mixture was heated to 75° C. for 15 hrs. The volatiles were evaporated and to this residue was added NaOMe/MeOH (3 mL) and stirred at room temperature for 15 hrs. This mixture was neutralized with acidic resin (Amberlite H+), filtered, concentrated and purified by silica gel column chromatography using a stepwise gradient of MeOH (2-8%) in CH2Cl2 to give 221 mg (0.78 mmol, 81%) of (2R,3S,4R,5R)-2-(6-Amino-9H-purin-9-yl)-4-fluoro-5-(hydroxymethyl)-3-methyltetrahydrofuran-3-ol.

1H NMR (200 MHz, CD3OD): δ 8.44 (s, 1H), 8.2 (s, 1H), 6.11 (d, J=3.6 Hz, 1H), 5.25 (d, J=5 Hz, 0.5H), 5.01 (d, J=5 Hz, 0.5H), 4.4-4.2 (m, 1H), 3.95 (m, 1H), 1.03 (s, 3H)

19F NMR (188 MHz, CD3OD): δ −216.71, −216.79, −217.00, −217.08

### Example 9

### 4-Amino-1-((2R,3S,4R,5R)-4-fluoro-3-hydroxy-5-(hydroxymethyl)-3-methyltetrahydrofuran-2-yl)pyrimidin-2(1H)-one

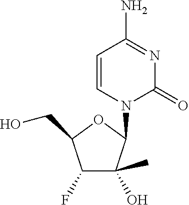 [SMILES: C[C@@]1(O)[C@H](F)[C@@H](CO)O[C@H]1n1ccc(N)nc1=O]

The compound of Example 9 was synthesized in two steps starting from commercially available N-benzoylcytosine and the sugar derivative, (2R,3S,4R,5R)-2-(6-amino-9H-purin-9-yl)-4-fluoro-5-(hydroxymethyl)-3-methyltetrahydrofuran-3-ol VI, prepared in Example 1.

### Step 1 Preparation of ((2R,3R,4S,5R)-5-(4-benzamido-2-oxopyrimidin-1(2H)-yl)-4-(benzoyloxy)-3-fluoro-4-methyltetrahydrofuran-2-yl)methyl benzoate

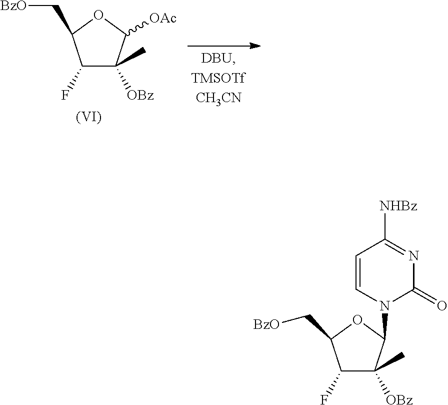

To the mixture of N-benzoylcytosine (285 mg, 1.32 mmol) and compound VI (500 mg, 1.20 mmol) in anhydrous CH3CN (10 mL) was added DBU (539 μL, 3.605 mmol) and cooled to 0° C. To this mixture TMSOTf (910 μL, 5.5 mmol) was added drop wise and heated to 70° C. for 12 hrs. The reaction mixture was diluted with EtOAc and washed with sat.aq.NaHCO3 and brine. The organic layers were dried with anhyd.Na2SO4, filtered and concentrated. The resulting residue was chromatographed on a column of silica gel using a stepwise gradient of ethyl acetate (20-40%) in hexane to obtain 338 mg (0.59 mmol, 49%) of ((2R,3R,4S,5R)-5-(4-benzamido-2-oxopyrimidin-1(2H)-yl)-4-(benzoyloxy)-3-fluoro-4-methyltetrahydrofuran-2-yl)methyl benzoate.

1H NMR (200 MHz, CDCl3): δ 8.2 (m, 4H), 7.9 (m, 3H), 7.7-7.4 (m, 10H), 6.55 (bs, 1H) 5.6 (bs, 0.5H), 5.35 (d, J=3 Hz, 0.5H), 4.8 (bs, 2H), 4.6 (m, 1H), 1.61 (s, 3H)

19F NMR (188 MHz, CDCl3): δ −193.73 (2) −194.81(2)

### Step 2 Preparation of 4-amino-1-((2R,3S,4R,5R)-4-fluoro-3-hydroxy-5-(hydroxymethyl)-3-methyltetrahydrofuran-2-yl)pyrimidin-2(1H)-one

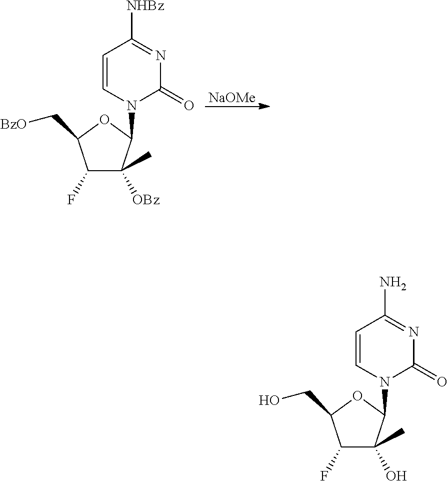

((2R,3R,4S,5R)-5-(4-Benzamido-2-oxopyrimidin-1(2H)-yl)-4-(benzoyloxy)-3-fluoro-4-methyltetrahydrofuran-2-yl)methyl benzoate (338 mg, 0.59 mmol) was taken in MeOH followed by addition of 2 mL of NaOMe/MeOH and stirred at room temperature for 48 hrs. This mixture was neutralized with acidic resin, filtered, concentrated and purified by silica gel column chromatography using a stepwise gradient of i-PrOH (20-34%) in CH2Cl2 to give 120 mg of 4-amino-1-((2R,3S,4R,5R)-4-fluoro-3-hydroxy-5-(hydroxymethyl)-3-methyltetrahydrofuran-2-yl)pyrimidin-2(1H)-one.

1H NMR (200 MHz, CD3OD): δ 8.0 (d, J=8 Hz, 1H), 6.01 (J=3 Hz, 1H), 5.89 (d, J=8 Hz, 1H), 4.9 (d, J=7 Hz, 0.5H), 4.6 (d, J=7 Hz, 0.5H), 4.2 (m, 1H), 3.8 (m, 2H), 1.1 (s, 3H)

19F NMR (188 MHz, CD3OD): δ −217.82, −217.9, −218.10, −218.17

### Example 10

### 1-((2R,3S,4R,5R)-4-Fluoro-3-hydroxy-5-(hydroxymethyl)-3-methyltetrahydrofuran-2-yl)pyrimidine-2,4(1H,3H)-dione

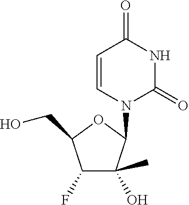 [SMILES: C[C@@]1(O)[C@H](F)[C@@H](CO)O[C@H]1n1ccc(=O)[nH]c1=O]

The compound of Example 10 was prepared in two steps starting from commercially available uracil and the sugar derivative ((2R,3R,4S)-5-acetoxy-4-(benzoyloxy)-3-fluoro-4-methyltetrahydrofuran-2-yl)methyl benzoate (VI) prepared as described in Example 1.

### Step 1 Preparation of ((2R,3R,4S,5R)-4-(benzoyloxy)-5-(2,4-dioxo-3,4-dihydropyrimidin-1(2H)-yl)-3-fluoro-4-methyltetrahydrofuran-2-yl)methyl benzoate

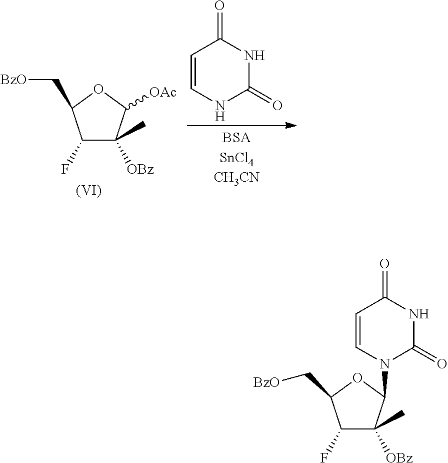

To a suspension of uracil (431 mg, 3.86 mmol) in anhydrous CH3CN (15 mL) was added N,O-bistrimethylsilyl acetamide (BSA) (940 μL, 3.85 mmol) and refluxed for 30 minutes. This mixture was cooled to room temperature followed by addition of compound VI (800 mg, 1.92 mmol) and SnCl4 (360 μL, 3.076 mmol) and further refluxed for 12 hrs. The reaction mixture was diluted with EtOAc and washed with sat.aq.NaHCO3 and brine. The organic layers were dried with anhyd.Na2SO4, filtered and concentrated. The resulting residue was chromatographed on a column of silica gel using a stepwise gradient of ethyl acetate (20-40%) in hexane to obtain 607 mg (1.3 mmol, 67%) of ((2R,3R,4S,5R)-4-(benzoyloxy)-5-(2,4-dioxo-3,4-dihydropyrimidin-1(2H)-yl)-3-fluoro-4-methyltetrahydrofuran-2-yl)methyl benzoate.

1H NMR (200 MHz, CDCl3): δ 10.1 (bs, 1H), 8.1 (m, 4H), 7.6-7.4 (m, 7H), 6.35 (s, 1H), 5.8 (d, J=8 Hz, 1H), 5.55 (bs, 0.5H), 5.22 (d, J=3 Hz, 0.5H), 4.71 (bs, 2H), 4.55 (m, 1H), 1.62 (s, 3H)

19F NMR (188 MHz, CDCl3): δ −189.93 (2), −190.73 (2)

### Step 2 Preparation of 1-((2R,3S,4R,5R)-4-fluoro-3-hydroxy-5-(hydroxymethyl)-3-methyltetrahydrofuran-2-yl)pyrimidine-2,4(1H,3H)-dione

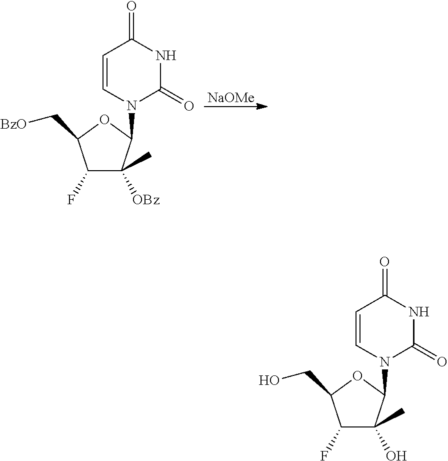

((2R,3R,4S,5R)-4-(benzoyloxy)-5-(2,4-dioxo-3,4-dihydropyrimidin-1(2H)-yl)-3-fluoro-4-methyltetrahydrofuran-2-yl)methyl benzoate (607 mg, 1.3 mmol) was taken in MeOH followed by addition of 2 mL of NaOMe/MeOH and stirred at room temperature for 36 hrs. This mixture was neutralized with acidic resin (Amberlite H+), filtered, concentrated and purified by silica gel column chromatography using a stepwise gradient of MeOH (2-8%) in CH2Cl2 to give 254 mg (0.98 mmol, 75%) of the deprotected nucleoside 1-((2R,3S,4R,5R)-4-fluoro-3-hydroxy-5-(hydroxymethyl)-3-methyltetrahydrofuran-2-yl)pyrimidine-2,4(1H,3H)-dione.

1H NMR (200 MHz, CD3OD): δ 8.01 (d, J=7 Hz, 1H), 5.92 (d, J=2.8 Hz, 1H), 5.7 (d, J=8 Hz, 1H), 4.9 (d, J=6 Hz, 0.5H), 4.6 (d, J=6 Hz, 0.5H), 4.2 (m, 1H), 3.8 (m, 2H), 1.23 (s, 3H)

19F NMR (188 MHz, CD3OD): δ −217.25, −217.33, −217.54, −217.61

### Example 11

### (2R,3S,4S,5R)-2-(2-Amino-6-methoxy-9H-purin-9-yl)-4-fluoro-5-(hydroxymethyl)-3-methyltetrahydrofuran-3-ol

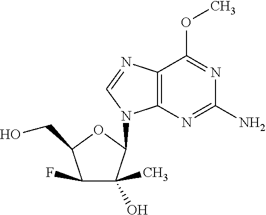 [SMILES: COc1nc(N)nc2c1ncn2[C@@H]1O[C@H](CO)[C@H](F)[C@@]1(C)O]

The compound of Example 11 was synthesized in multiple steps from the sugar derivative (3aR,5R,6R,6aR)-5-((tert-butyldiphenylsilyloxy)methyl)-2,2,6a-trimethyltetrahydrofuro[2,3-d][1,3]dioxol-6-yl 4-methylbenzenesulfonate (XVIII), prepared as described in PCT Int. Appl. (2010), WO 2010081082 A2, from commercially available 2-C-methyl ribonolactone (XIV). The synthesis of XVIII is also described in the general chemistry discussion above.

### Step 1 Preparation of (3aR,5R,6R,6aR)-5-(hydroxymethyl)-2,2,6a-trimethyltetrahydrofuro[2,3-d][1,3]dioxol-6-yl-4-methylbenzenesulfonate

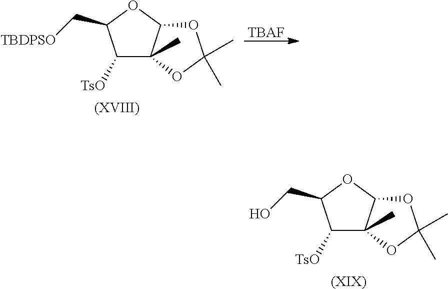

Compound XVIII was synthesized as described in PCT Int. Appl. (2010), WO 2010081082 A2. To an ice cooled solution of compound XVIII (21 gm, 0.035 mol) in 250 mL THF was added TBAF (53 mL, 0.053 mol) and stirred at room temperature for 4 hrs. The reaction mixture was diluted with EtOAc and washed with sat.aq.NaHCO3 and brine. The residue was purified by column chromatography to afford 12.6 gm (0.035 mol, 99%) of compound XIX.

1H NMR (200 MHz, CDCl3) δ 7.85 (d, J=8.1 Hz, 2H), 7.41 (d, J=8 Hz, 2H), 5.42 (s, 1H), 4.6 (d, J=8.8 Hz, 1H), 4.15 (m, 1H), 3.8 (m, 1H), 3.6-3.4 (m, 1H), 2.44 (s, 3H), 1.88 (m, 1H), 1.53 (s, 3H), 1.39 (s, 3H), 1.23 (s, 3H)

### Step 2 Preparation of (3aR,5R,6R,6aR)-5-(benzyloxymethyl)-2,2,6a-trimethyltetrahydrofuro[2,3-d][1,3]dioxol-6-yl 4-methylbenzenesulfonate

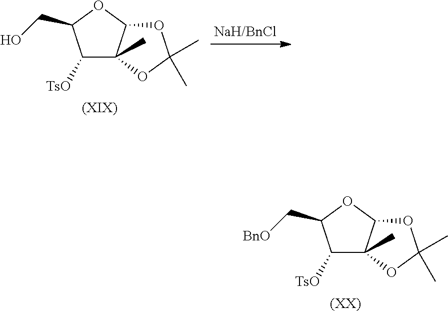

Compound XIX (12.5 gm, 0.035 mol) was dissolved in DMF was cooled to 0° C. and NaH (1.02 gm, 0.042 mol) was added to this solution and stirred for 30 min at 0° C. To this solution was added benzyl bromide (5.0 mL, 0.042 mol) and slowly warmed to room temperature and stirred for 6 hrs. The reaction mixture was diluted with EtOAc and washed with sat.aq.NH4Cl and brine. The organic layers were dried with anhyd.Na2SO4, filtered and concentrated and the residue was column chromatographed to give 14.2 gm (0.032 mol, 90%) of the benzylated compound XX.

1H NMR (200 MHz, CDCl3) δ 7.8 (d, J=8 Hz, 2H), 7.3 (m, 7H), 5.41 (s, 1H), 4.6 (m, 1H), 4.44 (bs, 2H), 4.25 (m, 1H), 3.7-3.3 (m, 2H), 2.4 (s, 3H), 1.5 (s, 3H), 1.41 (s, 3H), 1.25 (s, 3H).

### Step 3 Preparation of (3aR,5R,6S,6aS)-5-(benzyloxymethyl)-6-fluoro-2,2,6a-trimethyltetrahydrofuro[2,3-d][1,3]dioxole

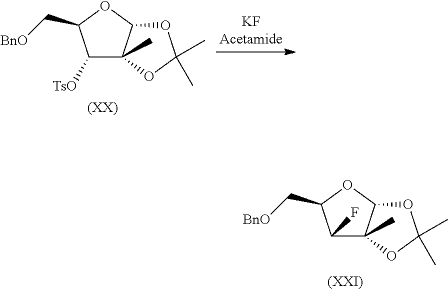

A mixture of compound XX (11.2 gm, 0.025 mol), KF (43.8 gm, 0.75 mol) and acetamide (100 gm) was heated to 210° C. for 5 hrs. The reaction mixture was cooled to 100° C. and poured into cool sat.aq. NaHCO3 and this mixture was diluted with EtOAc and washed with brine. The residue was purified by column chromatography to obtain 3.7 gm (0.0125 mol, 50%) of compound XXI and 4.3 gm (0.0096 mol, 38%) of unreacted starting material XX.

1H NMR (200 MHz, CDCl3) δ 7.35 (m, 5H), 5.6 (s, 1H), 4.8 (d, J=3 Hz, 0.5H), 4.62 (d, J=3 Hz, 0.5H), 4.58 (d, J=8 Hz, 2H), 4.5 (m, 1H), 3.7 (m, 2H), 1.6-1.4 (m, 9H)

19F NMR (188 MHz, CDCl3) δ −206.933, −207.087, −207.208, −207.346

### Step 4 Preparation of (3S,4S,5R)-5-(benzyloxymethyl)-4-fluoro-3-methyltetrahydrofuran-2,3-diol

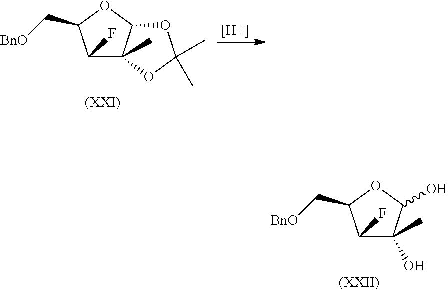

The compound XXI (2.68 gm, 0.009 mol)) was taken in 90% acetic acid and heated to 75° C. with catalytic amounts of Conc. H2SO4 for 6 days. The reaction mixture was washed with sat. aq. NaHCO3 (3×100 mL) and brine and the organic layers were dried, filtered and concentrated. The crude residue was purified by column chromatography to give 2.14 gm (0.0083 mol, 93%) of compound XXVII as a mixture of reducing sugars.

1H NMR (200 MHz, CDCl3): δ 7.4-7.1 (m, 8H), 5.2 (bs, 1H), 5.11 (bs, 0.3H), 4.8 (bs, 0.6H), 4.75-4.4 (m, 5H), 3.8-3.75 (m, 3H), 2.35 (bs, 1H), 2.11 (bs, 1H), 1.45 (bs, 1H), 1.4 (d, J=6 Hz, 3H)

19F NMR (188 MHz, CDCl3) δ −200.23, −200.311, −200.376, −200.40, −205.42, −205.7, −205.70, −205.85

### Step 5 Preparation of (3S,4S,5R)-5-(benzyloxymethyl)-4-fluoro-3-methyltetrahydrofuran-2,3-diyl dibenzoate

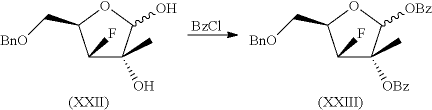

To the mixture of compound XXII (2.14 gm, 0.0083 mol) in pyridine was added benzoylchloride (2.91 mL, 0.025 mol) and 10 mol % of DMAP. The solution was heated to 75° C. for 36 hrs. The reaction mixture was washed with 1N HCl and extracted with EtOAc. The compound XXII (2.9 gm) was obtained as a mixture of anomers by purifying the crude residue by silica gel column chromatography.

1H NMR (200 MHz, CDCl3): δ 8.11-7.8 (m, 5H), 7.6-7.2 (m, 15H), 6.85 (s, 0.3H), 6.55 (s, 1H), 5.77 (d, J=6 Hz, 0.5H), 5.6 (d, J=6 Hz, 0.16H), 5.44 (d, J=6 Hz, 0.5H), 5.3 (d, J=6 Hz, 0.16H), 4.8-4.5 (m, 4H), 3.9-3.6 (m, 3H), 1.9 (d, J=5 Hz, 1H), 1.82 (d, J=8 Hz, 3H)

19F NMR (188 MHz, CDCl3) δ −205.53, −205.67, −205.80, −205.95, −209.46, −209.58, −209.75, −209.86

### Step 6 Preparation of (2R,3S,4S,5R)-2-(2-amino-6-chloro-9H-purin-9-yl)-5-(benzyloxymethyl)-4-fluoro-3-methyltetrahydrofuran-3-yl benzoate

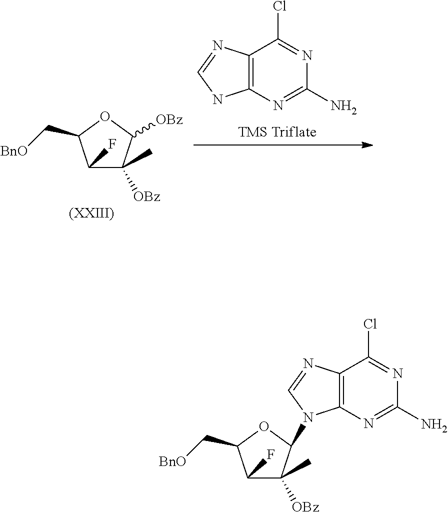

To benzoylated sugar derivative XXIII (1.2 gm, 2.5 mmol) in 25 mL of acetonitrile, was added 2-amino-6-chloropurine (526 mg, 3.1 mmol) and DBU (1.16 mL, 7.7 mmol) at room temperature. This mixture was cooled to 0° C. and TMStriflate (1.96 mL, 1.1 mmol) was added dropwise. The reaction mixture was warmed to room temperature and the heated to 65° C. for 15 hrs. The reaction mixture was washed with sat.aq.NaHCO3, brine and extracted with CH2Cl2. A column chromatographic purification afforded 0.98 gm (1.9 mmol, 76%) of (2R,3S,4S,5R)-2-(2-amino-6-chloro-9H-purin-9-yl)-5-(benzyloxymethyl)-4-fluoro-3-methyltetrahydrofuran-3-yl benzoate.

1H NMR (200 MHz, CDCl3): δ 8.18-7.96 (m, 2H), 7.91 (s, 1H), 7.63-7.2 (m, 9H), 6.58 (s, 1H), 5.77 (d, J=3 Hz, 0.5H), 5.44 (d, J=3 Hz, 0.5H), 5.27 (bs, 2H), 4.8-4.48 (m, 3H), 3.98-3.8 (m, 2H), 1.51 (s, 3H)

19F NMR (188 MHz, CDCl3) δ −204.33, −204.50, −204.61, −204.77

### Step 7 Preparation of (2R,3S,4S,5R)-2-(2-amino-6-methoxy-9H-purin-9-yl)-5-(benzyloxymethyl)-4-fluoro-3-methyltetrahydrofuran-3-ol

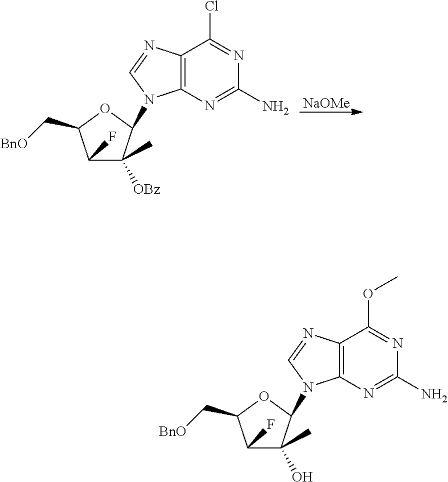

(2R,3S,4S,5R)-2-(2-Amino-6-chloro-9H-purin-9-yl)-5-(benzyloxymethyl)-4-fluoro-3-methyltetrahydrofuran-3-yl benzoate (0.85 mg, 1.66 mmol) of was dissolved in 6 mL of MeOH followed by addition of NaOMe dissolved in MeOH. This reaction mixture was stirred at room temp for 12 hrs. Neutralized with acidic resin and purified by column chromatography to give 0.59 gm (1.43 mmol, 86%) of the 5′-benzylated derivative (2R,3S,4S,5R)-2-(2-amino-6-methoxy-9H-purin-9-yl)-5-(benzyloxymethyl)-4-fluoro-3-methyltetrahydrofuran-3-ol.

1H NMR (200 MHz, CDCl3): δ 7.77 (s, 1H), 7.5 (bs, 5H), 6.19 (s, 1H), 5.2 (bs, 2H), 4.92 (d, J=3 Hz, 0.5H), 4.8 (m, 1H), 4.65 (d, J=3 Hz, 0.5H), 4.58 (s, 2H), 4.01 (s, 3H), 3.8 (m, 2H), 1.11 (s, 3H)

19F NMR (188 MHz, CDCl3) δ −202.66, −202.83, −202.94, −203.10

### Step 8 Preparation of (2R,3S,4S,5R)-2-(2-amino-6-methoxy-9H-purin-9-yl)-4-fluoro-5-(hydroxymethyl)-3-methyltetrahydrofuran-3-ol

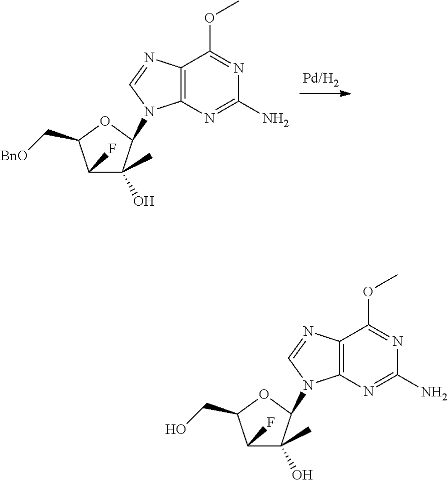

To a solution of (2R,3S,4S,5R)-2-(2-amino-6-methoxy-9H-purin-9-yl)-5-(benzyloxymethyl)-4-fluoro-3-methyltetrahydrofuran-3-ol (0.59 gm, 1.46 mmol) in CH3OH (20 mL) was added 100 mg of Pd/C with H2 (balloon pressure) and stirred at room temperature for 18 hrs. Then the Pd/C residue was filtered off and the volatiles were concentrated. The residue was purified by column chromatography with to afford 0.34 g (1.09 mmol, 75%) of the fully deprotected nucleoside, (2R,3S,4S,5R)-2-(2-amino-6-methoxy-9H-purin-9-yl)-4-fluoro-5-(hydroxymethyl)-3-methyltetrahydrofuran-3-ol.

1H NMR (200 MHz, CD3OD): δ 7.77 (s, 1H), 6.03 (s, 1H), 4.88 (d, J=3 Hz, 0.5H), 4.6 (d, J=3 Hz, 0.5H), 4.5 (m, 1H), 4.01 (s, 3H), 3.95 (m, 2H), 1.02 (s, 3H).

19F NMR (188 MHz, CD3OD) δ −203.25, −203.42, −203.53, −203.69

### Example 12

### (2S)-Benzyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

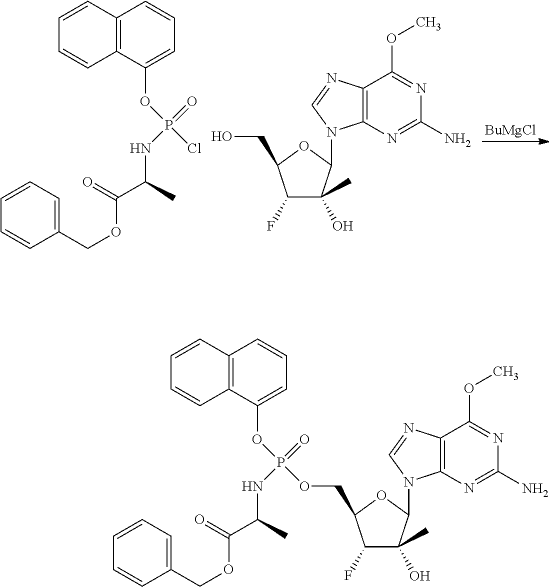

Using the procedure used for the synthesis of 5′-phosphoramidates of 2-amino-6-methoxy-9-(2-C-methyl-3-deoxy-3-fluoro-β-D-ribofuranosyl)purine, the nucleoside (80 mg, 0.256 mmol) in anhydrous THF (10 mL) was combined with (S)-2-[chloro-(naphthalen-1-yloxy)-phosphorylamino]-propionic acid benzyl ester (260 mg, 0.64 mmol), and t-Butyl magnesium chloride (0.64 mL, 0.64 mmol) After normal work-up the crude product was purified by column chromatography on silica gel twice using CH2Cl2/MeOH (95:5) and then with ethyl acetate/methanol (97:3) to give 78 mg of protide as off white solid.

1H NMR (200 MHz, CD3OD): δ 8.1 (m, 1H), 7.8-7.9 (m, 2H), 7.6-7.7 (m, 1H), 7.2-7.5 (m, 8H), 5.9 (m, 1H), 5.1-5.6 (m, 1H), 5.0 (m, 2H), 4.25-4.6 (m, 3H), 3.95-4.15 (m, 4H), 1.25-1.35 (m, 3H), 1.0 (d, J=9.6 Hz, 3H).

31P NMR (80 MHz, CD3OD): δ 5.39, 5.29

19F NMR (188 MHz, CD3OD): −213.49, −213.57, −213.78, −213.85, −214.07, −214.14, −214.35, −214.43.

### Example 13

### (2S)-2,4-Difluorobenzyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

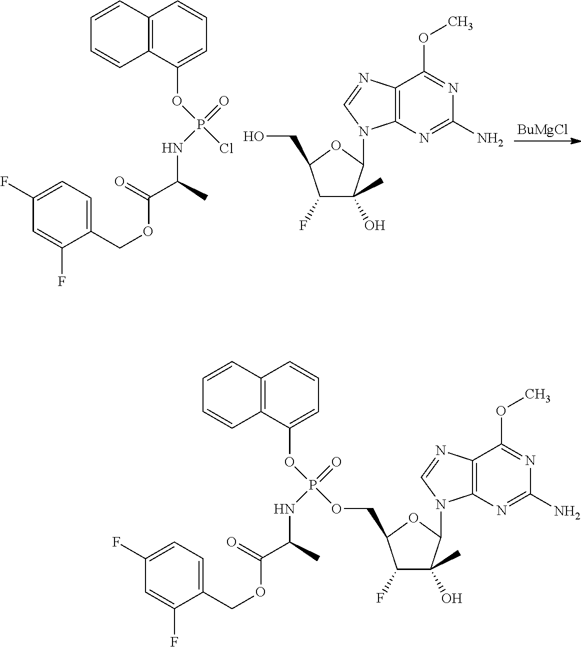

Using the general procedure for the synthesis of 5′-phosphoramidates of 2-amino-6-methoxy-(2-C-methyl-3-fluoro-β-D-ribofuranosyl)purine, the nucleoside (83 mg, 0.265 mmol) was dissolved in anhydrous THF (3 mL) and cooled to 0° C. To this mixture was added t-BuMgCl (0.663 mmol) and stirred for 15 min followed by addition of (2S)-2,4-difluorobenzyl 2-(chloro(naphthalene-1-yloxy)phosphorylamino)propanoate (291 mg 0.663 mmol) and stirred for 12 hrs at room temperature. Reaction mixture was washed with sat. aq.NH4Cl and extracted using CHCl3. First column chromatographic separation with CH2Cl2/MeOH (94:4) didn't yield pure compound. A second column chromatographic purification with EtOAc/MeOH (98:2) gave 73 mg of desired compound as an off white solid.

1H NMR (200 MHz, CD3OD): δ 8.08 (m, 1H), 7.87 (s, 1H), 7.8 (m, 1H), 7.63 (t, J=7.8 Hz, 1H), 7.5-7.2 (m, 5H), 6.7-6.85 (m, 2H), 5.89 (s, 1H), 5.4 (m, 1H), 5.01 (m, 2H), 4.7-4.35 (m, 3H), 4.1 (m, 1H), 3.97 (s, 3H), 1.3 (m, 3H), 1.01 (d, J=10 Hz, 3H)

31P NMR (80 MHz, CD3OD): δ 5.379, 5.228

19F NMR (188 MHz, CD3OD): δ −111.524 (m, 1F on phenyl ring), −115.803 (m, 1F on phenyl ring), −213.180, −213.258, −213.466, −213.550, −213.881, −213.958, −214.166, −214.244.

### Example 14

### (2S)-butyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

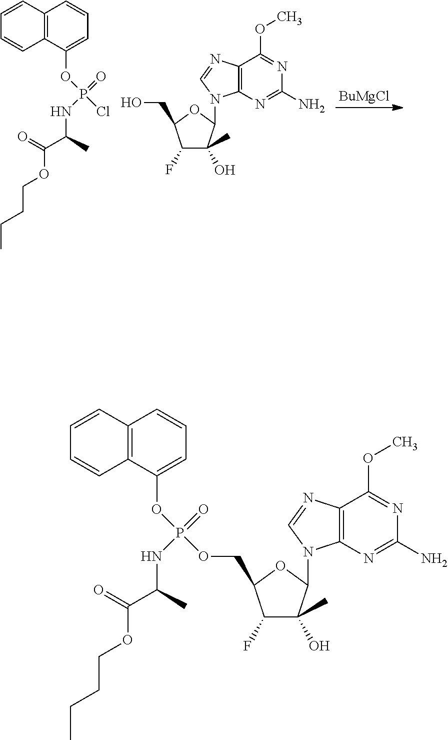

Using the general procedure for the synthesis of 5′-phosphoramidates of 2-amino-6-methoxy-(2-C-methyl-3-fluoro-β-D-ribofuranosyl)purine, the nucleoside (77 mg, 0.246 mmol) was dissolved in anhydrous THF (3 mL) and cooled to 0° C. To this mixture was added t-BuMgCl (0.615 mmol) and stirred for 15 min followed by addition of (2S)-butyl 2-(chloro(naphthalen-1-yloxy)phosphorylamino)propanoate (226.9 mg 0.615 mmol) and stirred for 12 hrs at room temperature. Reaction mixture was washed with sat. aq.NH4Cl and extracted with CHCl3. A column chromatographic separation with CH2Cl2/MeOH (94:4) gave 51 mg of desired compound as an off white solid.

1H NMR (200 MHz, CD3OD): δ 8.11 (m, 1H), 7.91 (s, 1H), 7.87 (m, 1H), 7.65 (m, 1H), 7.6-7.28 (m, 4H), 5.96 (s, 1H), 5.3 (m, 1H), 4.58 (m, 3H), 4.01 (s, 3H), 3.91 (m, 2H), 1.5 (m, 2H), 1.22 (m, 6H), 1.10 (d, J=10 Hz, 3H), 0.81 (m, 3H)

31P NMR (80 MHz, CD3OD): δ 5.470, 5.221

19F NMR (188 MHz, CD3OD): δ−213.459, −213.543, −213.751, −213.829, −213.945, −213.023, −213.231, −213.309

### Example 15

### (2S)-3,3-Dimethylbutyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

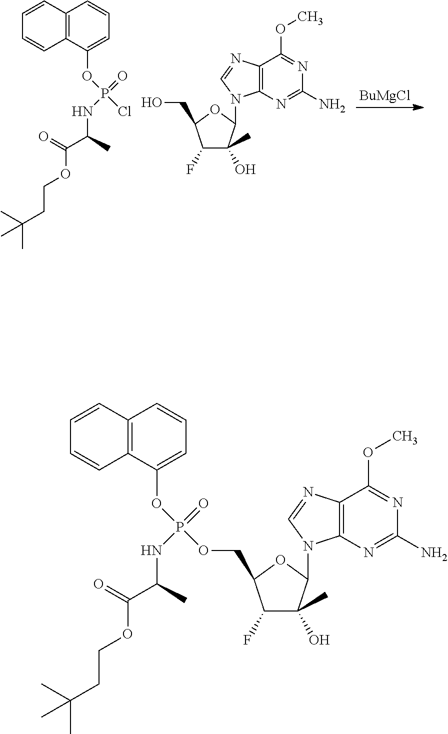

The nucleoside 2-amino-6-methoxy-(2-C-methyl-3-fluoro-β-D-ribofuranosyl)purine (83 mg, 0.265 mmol) was dissolved in 3 mL of anhydrous THF and cooled to 0° C. To this solution was added t-BuMgCl (0.66 mmol, 0.66 mL) and stirred for another 15 min followed by addition of (2S)-3,3-dimethylbutyl 2-(chloro(naphthalen-1-yloxy)phosphorylamino)propanoate (263 mg, 0.66 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 65 mg of desired compound.

1H NMR (200 MHz, CD3OD): δ 8.11 (m, 1H), 7.91 (s, 1H), 7.85 (m, 1H), 7.6 (m, 1H), 7.57-7.23 (m, 4H), 5.91 (s, 1H), 5.4 (m, 1H), 4.58 (m, 3H), 4.00 (s, 3H), 3.94 (m, 3H), 1.28 (m, 5H), 1.01 (d, J=10 Hz, 3H), 0.82 (s, 6H), 0.83 (s, 3H)

31P NMR (80 MHz, CD3OD): δ 5.462, 5.145

19F NMR (188 MHz, CD3OD): δ −213.550, −213.628, −213.835, −213.913, −214.114, −214.192, −214.406, −214.484

### Example 16

### (2S)-Neopentyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

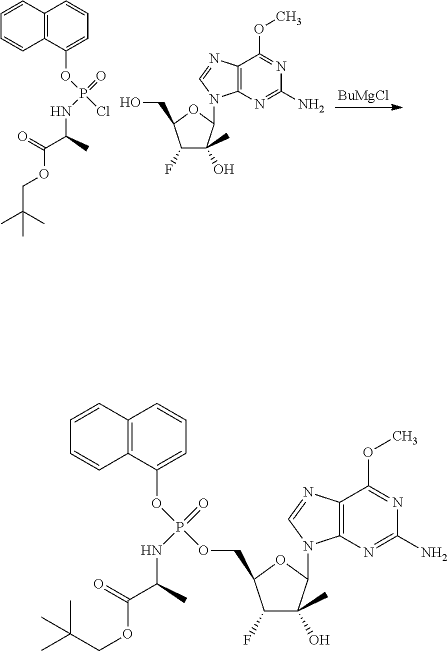

Using the procedure used for the synthesis of 5′-phosphoramidates of 2-amino-6-methoxy-9-(2-C-methyl-3-deoxy-3-fluoro-β-D-ribofuranosyl)purine, the nucleoside (100 mg, 0.319 mmol) in anhydrous THF (10 mL) was combined with (S)-2-[Chloro-(naphthalen-1-yloxy)-phosphorylamino]-propionic acid 2,2-dimethyl propyl ester (0.305 mg, 0.798 mmol), and t-Butyl magnesium chloride (0.798 mL, 0.798 mmol). After normal work-up the crude product was purified by column chromatography on silica gel using CH2Cl2/MeOH (97:3) to give 48 mg of protide in 17% yield as light yellow solid.

1H NMR (200 MHz, CD3OD): δ 8.0-8.2 (m, 1H), 7.78-7.9 (m, 2H), 7.6-7.75 (m, 1H), 7.2-7.4 (m, 4H), 5.9 (t, J=2.6 Hz, 1H), 5.1-5.6 (dd, J=7.4 Hz, J=7.2 Hz 1H), 4.4-1.7 (m, 3H), 4.1-4.0 (m, 4H), 3.6-3.8 (dq, J=3.8 Hz, 2H), 1.25-1.35 (m, 3H), 1.0 (d, J=7.4 Hz, 3H), 0.85 (d, J=1.4 Hz, 9H).

31P NMR (80 MHz, CD3OD): δ 5.43, 5.26

19F NMR (188 MHz, CD3OD): −213.63, −213.71, −213.93, −214.0, −214.21, −214.23.

### Example 17

### (2S)-Cyclopentyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

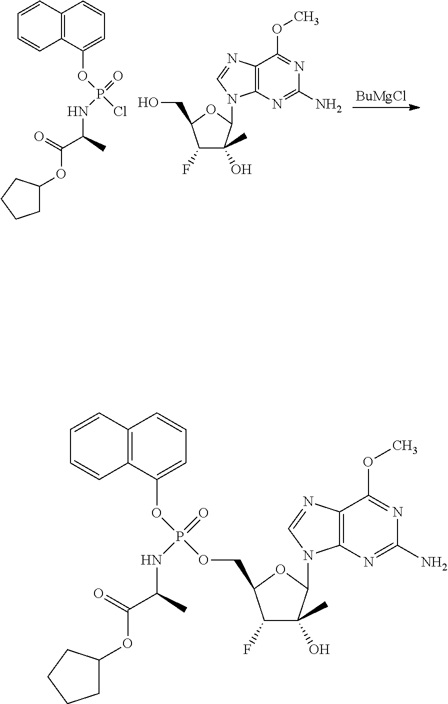

The nucleoside 2-amino-6-methoxy-(2-C-methyl-3-fluoro-β-D-ribofuranosyl)purine (80 mg, 0.255 mmol) was dissolved in 3 mL of anhydrous THF and cooled to 0° C. To this solution was added t-BuMgCl (0.64 mmol, 0.64 mL) and stirred for another 15 min followed by addition of (2S)cyclopentyl 2-(chloro(naphthalen-1-yloxy)phosphorylamino)propanoate (243 mg, 0.64 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 63 mg of pure protide.

1H NMR (200 MHz, CD3OD): δ 8.11 (m, 1H), 7.97 (s, 1H), 7.80 (m, 1H), 7.61 (m, 1H), 7.41 (m, 4H), 5.89 (s, 1H), 5.4 (m, 1H), 4.62 (m, 3H), 3.99 (s, 3H), 3.84 (m, 1H), 1.81-1.39 (m, 8H), 1.24 (m, 3H), 1.01 (d, J=10 Hz, 3H)

31P NMR (80 MHz, CD3OD): δ 5.553, 5.281

19F NMR (188 MHz, CD3OD): δ −213.018, −213.096, −213.304, −213.375, −213.582, −213.654

### Example 18

### (2S)-Cyclohexyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

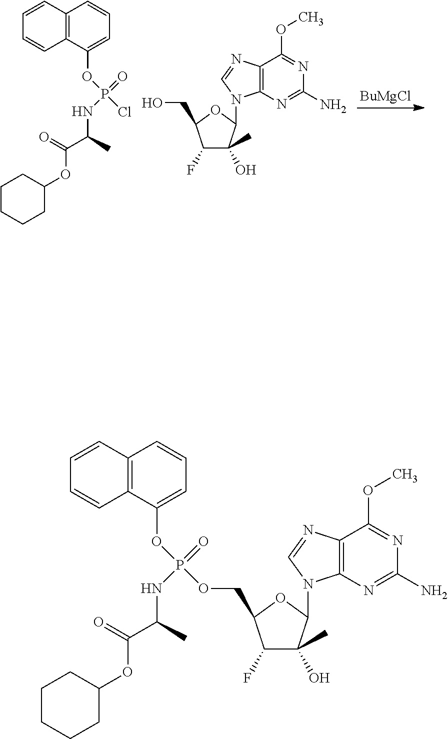

The nucleoside 2-amino-6-methoxy-(2-C-methyl-3-fluoro-β-D-ribofuranosyl)purine (83 mg, 0.265 mmol) was dissolved in 3 mL of anhydrous THF and cooled to 0° C. To this solution was added t-BuMgCl (0.66 mmol, 0.66 mL) and stirred for another 15 min followed by addition of (2S) cyclohexyl 2-(chloro(naphthalen-1-yloxy)phosphorylamino)propanoate (261 mg, 0.66 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 43 mg of desired compound.

1H NMR (200 MHz, CD3OD): δ 8.1 (m, 1H), 7.96 (s, 1H), 7.8 (m, 2H), 7.6-7.18 (m, 4H), 5.92 (s, 1H), 5.4 (m, 1H), 4.56 (m, 3H), 3.99 (s, 3H), 3.92 (m, 1H), 3.58 (m, 1H), 1.82-1.15 (m, 10H), 1.32 (m, 3H), 1.01 (d, J=10 Hz, 3H)

31P NMR (80 MHz, CD3OD): δ 5.485, 5.259

19F NMR (188 MHz, CD3OD): δ 213.291, −213.368, −213.576, −213.822, −213.894

### Example 19

### (2S)-Tetrahydro-2H-pyran-4-yl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

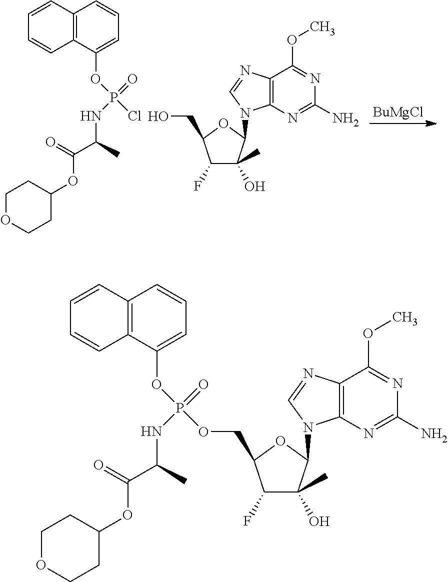

Using the general procedure for the synthesis of 5′-phosphoramidates of 2-amino-6-methoxy-(2-C-methyl-3-fluoro-β-D-ribofuranosyl)purine, the nucleoside (80 mg, 0.255 mmol) was dissolved in anhydrous THF (3 mL) and cooled to 0° C. To this mixture was added t-BuMgCl (0.639 mmol) and stirred for 15 min followed by addition of (2S)-tetrahydro-2H-pyran-4-yl 2-(chloro(naphthalene-1-yloxy)phosphoryl-amino)propanoate (253.6 mg 0.639 mmol) and stirred for 12 hrs at room temperature. Reaction mixture was washed with sat. aq. NH4Cl and extracted with CHCl3. First column chromatographic separation with CH2Cl2/MeOH (94:4) didn't yield pure compound. A second column chromatographic purification with EtOAc/MeOH (98:2) gave 82 mg of desired compound as an off white solid.

1H NMR (200 MHz, CD3OD): δ 8.12 (m, 1H), 7.87 (s, 1H), 7.84 (m, 1H), 7.65 (t, J=8 Hz, 1H), 7.46 (m, 4H), 5.8 (s, 1H), 5.4 (m, 1H), 4.8 (m, 1H), 4.6 (m, 3H), 3.99 (s, 3H), 3.96 (m, 1H), 3.75 (m, 2H), 3.44 (m, 2H), 1.74 (m, 2H), 1.51 (m, 2H), 1.31 (t, J=10 Hz, 3H), 1.02 (d, J=10 Hz, 3H)

31P NMR (80 MHz, CD3OD): δ 5.440, 5.183

19F NMR (188 MHz, CD3OD): δ −213.783, −213.861, −214.069, −214.134, −214.198, −214.412, −214.490

### Example 20

### (2S)-Neopentyl 2-((((2R,3R,4S,5R)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate

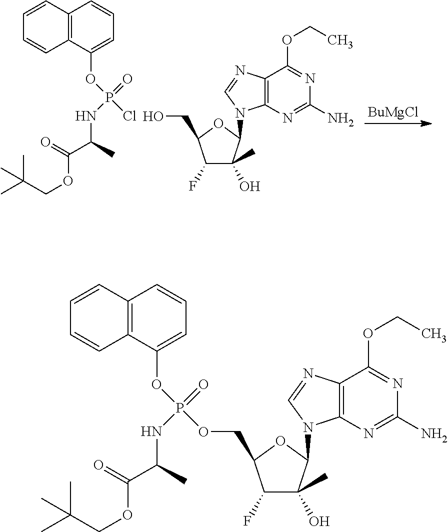

The nucleoside 2-amino-6-ethoxy-(2-C-methyl-3-fluoro-β-D-ribofuranosyl)purine (70 mg, 0.214 mmol) was dissolved in 7 mL of anhydrous THF and cooled to 0° C. To this solution was added t-BuMgCl (0.535 mmol, 0.535 mL) and stirred for another 15 min followed by addition of (2S)-3,3-dimethylpropyl 2-(chloro(naphthalen-1-yloxy)phosphorylamino)propanoate (205 mg, 0.535 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 57 mg of desired compound.

1H NMR (200 MHz, CD3OD): δ 8.11 (m, 1H), 7.84 (s, 1H), 7.80 (m, 1H), 7.58 (m, 1H), 7.39 (, 4H), 5.89 (s, 1H), 5.4 (m, 1H), 4.62 (m, 2H), 4.48 (q, J=6.8 Hz, 2H), 4.01 (m, 1H), 3.67 (dq, J=16.6 Hz, J=2.2 Hz, 2H), 1.36 (m, 6H), 1.02 (m, J=10 Hz, 3H), 0.81 (s, 9H)

31P NMR (80 MHz, CD3OD): δ 5.400, 5.259

19F NMR (188 MHz, CD3OD): δ −213.569, −213.647, −213.829, −213.907, −214.108, −214.185

### Example 21

### (2S)-Cyclopentyl 2-((((2R,3R,4S,5R)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate

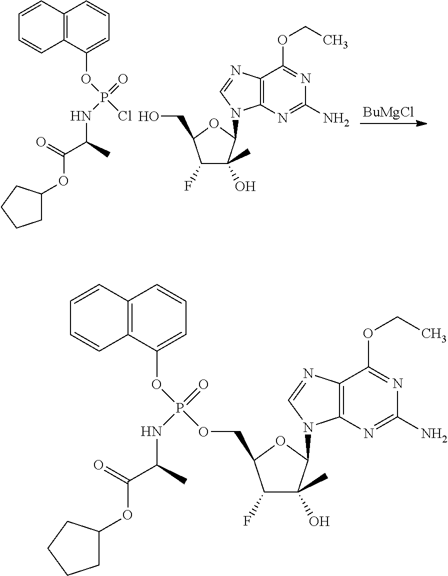

The nucleoside 2-amino-6-ethoxy-(2-C-methyl-3-fluoro-β-D-ribofuranosyl)purine (70 mg, 0.214 mmol) was dissolved in 8 mL of anhydrous THF and cooled to 0° C. To this solution was added t-BuMgCl (0.535 mmol, 0.535 mL) and stirred for another 15 min followed by addition of (2S) cyclopentyl 2-(chloro(naphthalen-1-yloxy)phosphorylamino)propanoate (204 mg, 0.535 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 43 mg of pure protide.

1H NMR (200 MHz, CD3OD): δ 8.11 (m, 1H), 7.86 (s, 1H), 7.80 (m, 1H), 7.61 (m, 1H), 7.41 (m, 4H), 5.89 (s, 1H), 5.4 (m, 1H), 5.01 (m, 1H), 4.6 (m, 2H), 4.44 (q, J=7 Hz, 2H), 3.94 (m, 1H), 1.82-1.37 (m, 8H), 1.27 (m, 3H), 1.03 (d, J=10 Hz, 3H)

31P NMR (80 MHz, CD3OD): δ 5.523, 5.228

19F NMR (188 MHz, CD3OD): δ −213.401, −213.479, −213.693, −213.770, −213.842, −214.056, −214.134

### Example 22

### (2S)-Cyclohexyl 2-((((2R,3R,4S,5R)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate

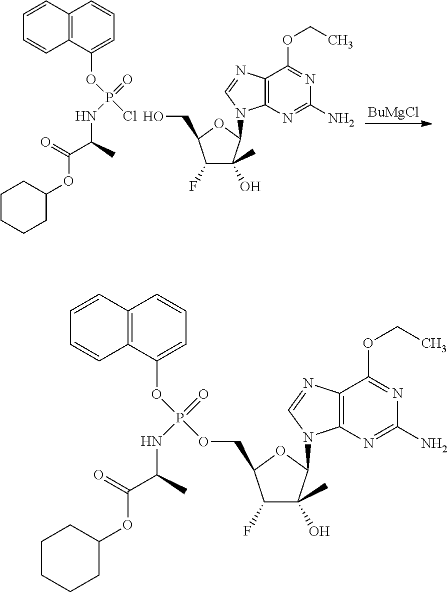

The nucleoside 2-amino-6-ethoxy-(2-C-methyl-3-fluoro-β-D-ribofuranosyl)purine (70 mg, 0.214 mmol) was dissolved in 8 mL of anhydrous THF and cooled to 0° C. To this solution was added t-BuMgCl (0.535 mmol, 0.535 mL) and stirred for another 15 min followed by addition of (2S)cyclohexyl 2-(chloro(naphthalen-1-yloxy)phosphorylamino)propanoate (211 mg, 0.535 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 55 mg of desired compound.

1H NMR (200 MHz, CD3OD): δ 8.12 (m, 1H), 7.84 (s, 1H), 7.77 (m, 1H), 7.61 (m, 1H), 7.39 (m, 4H), 5.90 (s, 1H), 5.4 (m, 1H), 4.62 (m, 4H), 4.44 (q, J=7 Hz, 2H), 4.02 (m, 1H), 1.6 (m, 4H), 1.33 (m, 12H), 1.01 (d, J=10 Hz, 3H)

31P NMR (80 MHz, CD3OD): δ 5.508, 5.243

19F NMR (188 MHz, CD3OD): δ −213.362, −213.440, −213.693, −213.725, −213.764, −213.978, −214.056

### Example 23

### (2S)-Neopentyl 2-((((2R,3R,4S,5R)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate

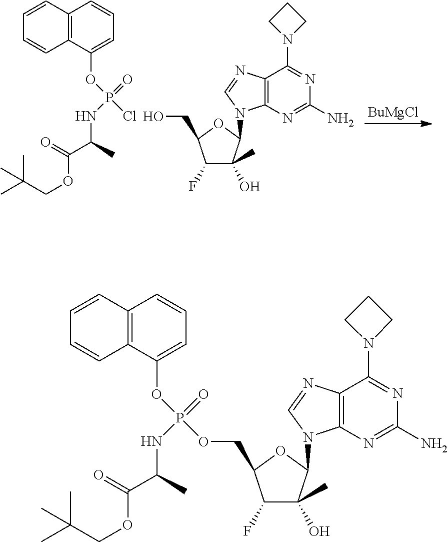

The nucleoside 2-amino-6-(azetidin-1yl)-(2-C-methyl-3-fluoro-β-D-ribofuranosyl)purine (70 mg, 0.207 mmol) was dissolved in 7 mL of anhydrous THF and cooled to 0° C. To this solution was added t-BuMgCl (0.517 mmol, 0.517 mL) and stirred for another 15 min followed by addition of (2S)-neopentyl 2-(chloro(naphthalen-1-yloxy)phosphorylamino)propanoate (198 mg, 0.517 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 80 mg of desired compound.

1H NMR (200 MHz, CD3OD): δ 8.11 (m, 1H), 7.89 (s, 1H), 7.86 (m, 1H), 7.65 (m, 1H), 7.38 (m, 4H), 5.82 (t, J=2.8 Hz, 1H), 5.2 (m, 1H), 4.55 (m, 5H), 4.28 (m, 1H), 4.11 (m, 1H), 3.71 (m, 2H), 2.48 (m, 2H), 1.28 (m, 3H), 1.01 (d, J=10 Hz, 3H), 0.81 (s, 6H), 0.82 (s, 3H)

31P NMR (80 MHz, CD3OD): δ 5.538

19F NMR (188 MHz, CD3OD): δ −214.106

### Example 24

### (2S)-Cyclopentyl 2-((((2R,3R,4S,5R)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate

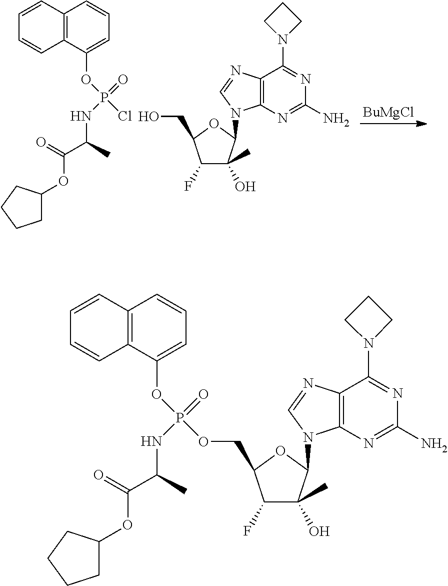

The nucleoside 2-amino-6-(azetidin-1yl)-(2-C-methyl-3-fluoro-β-D-ribofuranosyl)purine (70 mg, 0.207 mmol) was dissolved in 8 mL of anhydrous THF and cooled to 0° C. To this solution was added t-BuMgCl (0.517 mmol, 0.517 mL) and stirred for another 15 min followed by addition of (2S)cyclopentyl 2-(chloro(naphthalen-1-yloxy)phosphorylamino)propanoate (198 mg, 0.517 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 34 mg of pure protide.

1H NMR (200 MHz, CD3OD): δ 8.14 (m, 1H), 7.84 (m, 1H), 7.77 (s, 1H), 7.62 (m, 1H), 7.44 (m, 3H), 7.38 (m, 1H), 5.84 (t, J=3 Hz, 1H), 5.44 (m, 1H), 5.01 (m, 1H), 4.58 (m, 2H), 4.3 (m, 3H), 3.9 (m, 1H), 2.43 (m, 2H), 1.82-1.4 (bm, 10H), 1.25 (m, 3H), 1.03 (d, J=10 Hz, 3H)

31P NMR (80 MHz, CD3OD): δ 5.455, 5.183

19F NMR (188 MHz, CD3OD): δ −213.615, −213.693, −213.907, −213.971, −214.244, −214.322

### Example 25

### (2S)-Cyclohexyl 2-((((2R,3R,4S,5R)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate

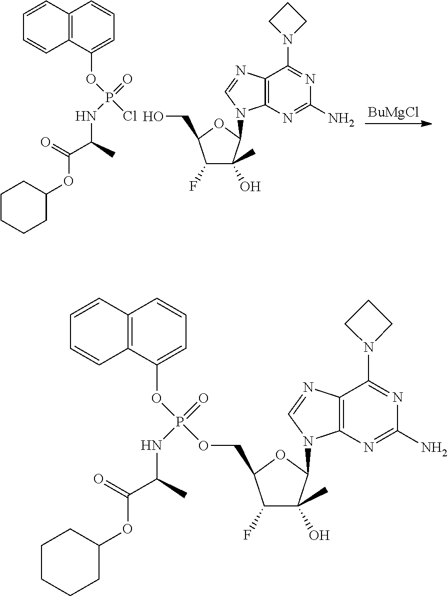

The nucleoside 2-amino-6-(azetidin-1yl)-(2-C-methyl-3-fluoro-β-D-ribofuranosyl)purine (70 mg, 0.207 mmol) was dissolved in 8 mL of anhydrous THF and cooled to 0° C. To this solution was added t-BuMgCl (0.517 mmol, 0.517 mL) and stirred for another 15 min followed by addition of (2S)cyclohexyl 2-(chloro(naphthalen-1-yloxy)phosphorylamino)propanoate (204 mg, 0.517 mmol) at 0° C. and stirred at room temperature for 15 hrs. The reaction mixture was washed with sat.aq.NH4Cl and extracted with CH2Cl2. A column chromatography separation with gradient mixture of CH3OH:CH2Cl2 (3:97) obtained 38 mg of desired compound.

1H NMR (200 MHz, CD3OD): δ 8.09 (m, 1H), 7.83 (m, 1H), 7.78 (s, 1H), 7.64 (m, 1H), 7.58-7.25 (m, 4H), 5.84 (t, J=2.56 Hz, 1H), 5.4 (m, 1H), 4.59 (m, 4H), 4.37 (m, 4H), 3.94 (m, 1H), 2.19 (m, 2H), 1.61 (m, 4H), 1.33 (m, 10H), 1.02 (d, J=10 Hz, 3H)

31P NMR (80 MHz, CD3OD): δ 5.455, 5.198

19F NMR (188 MHz, CD3OD): δ −213.699, −213.777, −213.991, −214.069, −214.283, −214.367

### Example 26

### (2S)-Benzyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate

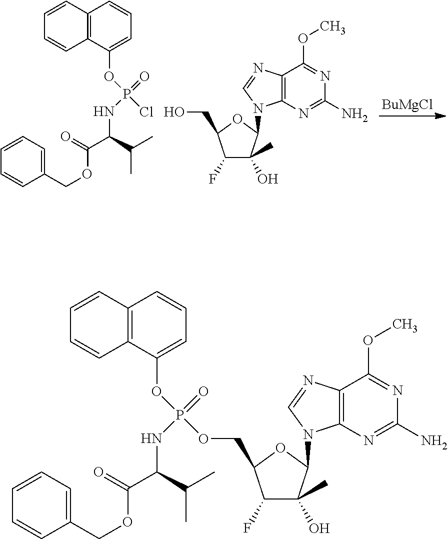

Using the procedure used for the synthesis of 5′-phosphoramidates of 2-amino-6-methoxy-9-(2-C-methyl-3-deoxy-3-fluoro-β-D-ribofuranosyl)purine, the nucleoside (48 mg, 0.154 mmol) in anhydrous THF (5 mL) was combined with (S)-2-[chloro-(naphthalen-1-yloxy)-phosphorylamino]-3-methyl butyric acid benzyl ester (156 mg, 0.384 mmol), and t-Butyl magnesium chloride (0.39 mL, 0.384 mmol). After normal work-up the crude product was purified by column chromatography on silica gel using CH2Cl2/MeOH (96:4) to give 42 mg of protide as light yellow solid.

1H NMR (200 MHz, CD3OD): δ 8.1-8.2 (m, 1H), 7.76-7.85 (m, 2H), 7.6 (m, 1H), 7.1-7.45 (m, 9H), 5.89 (d, J=2.6 Hz, 1H), 5.06-5.67 (m, 1H), 4.96 (m, 2H), 4.6-4.8 (m, 3H), 4.0 (d, J=3.0 Hz, 3H), 3.6-3.8 (m, 1H), 2.0 (m, 1H), 0.7-1.1 (m, 9H).

31P NMR (80 MHz, CD3OD): δ 6.26, 6.04.

19F NMR (188 MHz, CD3OD): −213.3, −213.39, −213.44, −213.52, −213.56, −213.68, −213.73, −213.81.

Phosphordiamidates of the general structure of Example 27 can be synthesized by a variety of methods including general Methods A-D described below.

### Method A: Symmetrical Phosphoramidates Using Triethyl Phosphate and Phosphoryl Chloride

To a solution of the nucleoside (1 equiv) in anhydrous triethyl phosphate is added phosphoryl chloride (2 equiv) at 0° C. The reaction mixture is stirred 24 h at 5° C.

Anhydrous dichloromethane is added to the reaction mixture followed by amino acid ester (5 equiv) and diisopropylethylamine (10 equiv) at 0° C. After stirring at 5° C. for 5 days, water is added and the layers are separated. The aqueous phase is extracted with dichloromethane and the organic phase washed with brine. The combined organic layers are dried over anhyd sodium sulfate, filtered and evaporated to dryness. The resulting residue is purified by silica gel column chromatography using as eluent a gradient of methanol in dichloromethane. A subsequent repurification, if necessary, is accomplished either by preparative HPLC (gradient of methanol in water) or preparative TLC.

### Method B: Symmetrical Phosphoramidates Using THF and Phosphoryl Chloride

To a suspension of nucleoside (1 equiv) in anhyd tetrahydrofuran is added triethylamine (1.2 equiv). After stirring for 30 min at room temperature, phosphoryl chloride (1.2 equiv) is added dropwise at −78° C. The reaction mixture is stirred 30 min at −78° C. then allowed to warm to room temperature over 30 min. Anhydrous dichloromethane is added, followed by amino acid ester (5 equiv) and triethylamine (10 equiv) at −78° C. After stirring at room temperature for 20 h, water is added and the layers are separated. The aqueous phase is extracted with dichloromethane and the organic phase washed with brine. The combined organic layers are dried over anhyd sodium sulfate, filtered and evaporated to dryness. The resulting residue is purified by silica gel column chromatography using as eluent a gradient of methanol in dichloromethane. In some cases, a subsequent repurification is necessary either by preparative HPLC (gradient of methanol in water) or preparative TLC.

Method C: Asymmetrical Phosphodiamidates with Phosphorus Chloride

To a suspension of the nucleoside (1 equiv) in anhydrous tetrahydrofuran, is added triethylamine (1.2 equiv). After stirring for 30 min at room temperature, phosphoryl chloride (1.2 equiv) is added dropwise at −78° C. The reaction mixture is stirred 30 min at −78° C. then allowed to warm to room temperature over 30 min. Anhydrous dichloromethane is added, followed by a first amino acid ester or amine (1 equiv) and triethylamine (2 or 1 equiv respectively) at −78° C. The solution is warmed to room temperature and monitored by 31P NMR. When NMR indicates completion of the reaction (no starting material, presence of mono-substituted product) a second amino acid ester or amine (5 equiv) is added followed by the addition of triethylamine (10 or 5 equiv respectively) at −78° C. After stirring at room temperature for 20 h, water is added and the layers are separated. The aqueous phase is extracted with dichloromethane and the organic phase is washed with brine. The combined organic layers are dried over anhydrous sodium sulfate, filtered and evaporated to dryness. The resulting residue is purified by silica gel column chromatography using as eluent a gradient of methanol in chloroform (0-5%).

### Method D: Asymmetrical Phosphodiamidates Using Amino Acid Ester Phosphodichloridate

To a suspension of the nucleoside (1 equiv) in anhydrous tetrahydrofuran is added triethylamine (1.2 equiv). After stirring for 30 min at room temperature an amino acid ester phosphodichloridate (2 equiv) is added. After stirring at room temperature for 20 h, the solution is cooled to −78° C. and a primary amine is added (5 equiv) followed by triethylamine (5 equiv). The solution is warmed to room temperature and stirred for 20 h. Water is added and the layers are separated. The aqueous phase is extracted with dichloromethane and the organic phase washed with brine. The combined organic layers are dried over anhydrous sodium sulfate, filtered and evaporated to dryness. The resulting residue is purified by silica gel column chromatography using as eluent a gradient of methanol in chloroform (0-5%)

### Example 27

### (2S,2′S)-Neopentyl 2,2′-((((2R,3R,4S,5R)-5-(2-amino-6-methoxy-9H-purin-9-yl)-3-fluoro-4-hydroxy-4-methyltetrahydrofuran-2-yl)methoxy)phosphoryl)bis(azanediyl)dipropanoate

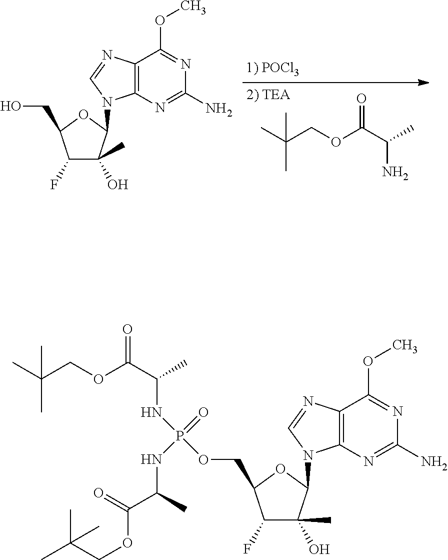 [SMILES: COc1nc(N)nc2c1ncn2[C@@H]1O[C@H](CO)[C@@H](F)[C@@]1(C)O ; C[C@H](N)C(=O)OCC(C)(C)C ; COc1nc(N)nc2c1ncn2[C@@H]1O[C@H](COP(=O)(N[C@@H](C)C(=O)OCC(C)(C)C)N[C@@H](C)C(=O)OCC(C)(C)C)[C@@H](F)[C@@]1(C)O]

Using general Method B for preparing phosphordiamidates, the nucleoside 2-amino-6-methoxy-9-(2-C-methyl-3-deoxy-3-fluoro-β-D-ribofuranosyl)purine (210 mg, 0.671 mmol) in anhydrous THF (10 mL) was treated with POCl3 (0.062 mL, 0.671 mmol) at rt. The solution was cooled to −78° C. and TEA (0.093 mL, 0.67 mmol) was added, and the temperature was raised to RT and stirred for 30 min. To this reaction mixture was added the HCl salt of (S)-2-Amino propionic acid-2, 2 dimethyl propyl ester (525 mg, 2.68 mmol), which was dissolved in DCM, cooled to −78° C. and treated with TEA (0.75 mL, 5.386 mmol). The resulting mixture was stirred at RT overnight. After normal work-up the crude product was purified by column chromatography on silica gel using CH2Cl2/MeOH (96:4) to give 25 mg of protide as white solid.

1H NMR (200 MHz, CD3OD): δ 7.93 (s, 1H), 5.91 (d, J=2.8 Hz, 1H), 5.6 (d, J=7.0 Hz, 0.5H), 5.3 (d, J=6.8 Hz, 0.5H), 4.3-4.5 (m, 3H), 3.6-4.0 (m, 11H), 1.3-1.4 (t, J=7.6 Hz, 6H), 1.1 (s, 3H), 0.9-1.0 (m, 18H).

31P NMR (80 MHz, CD3OD): δ 15.06

19F NMR (188 MHz, CD3OD): −214.36, −214, 43, −214.65, −214.72

### Example 28

### (2S)-Benzyl 2-((((2R,3S,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

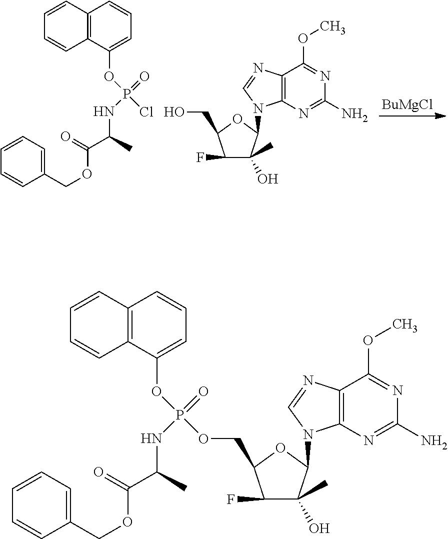

The nucleoside (2R,3S,4S,5R)-2-(2-amino-6-methoxy-9H-purin-9-yl)-4-fluoro-5-(hydroxymethyl)-3-methyltetrahydrofuran-3-ol (75 mg, 0.239 mmol) was dissolved in anhydrous THF (8 mL) and cooled to 0° C. followed by addition of t-butyl magnesium chloride (0.479 mL, 0.479 mmol) and stirred for 15 min. To this reaction mixture was added with (S)-2-[chloro-(naphthalen-1-yloxy)-phosphorylamino]-propanoic acid benzyl ester (274 mg, 0.638 mmol) and stirred at room temperature for 15 hrs. The reaction mixture was diluted with CH2Cl2 and washed with sa.aq.NH4Cl. The residue was purified by column chromatography to yield 58 mg of (2S)-benzyl 2-((((2R,3S,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

0.1H NMR (200 MHz, CD3OD): δ 8.2 (m, 1H), 7.9-7.6 (m, 3H), 7.6-7.3 (m, 5H), 7.2 (bs, 5H), 0.04 (d, J=2.4 Hz, 1H), 5.02 (m, 2H), 4.5 (m, 4H), 4.02 (m, 5H), 1.3 (m, 6H), 1.01 (bs, 3H)

19F NMR (188 MHz, CD3OD) δ −202.937, −203.091, −203.213, −203.375

31P NMR (80 MHz, CD3OD): δ 5.243, 5.108

### Example 29

### (2S)-Neopentyl 2-((((2R,3S,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate

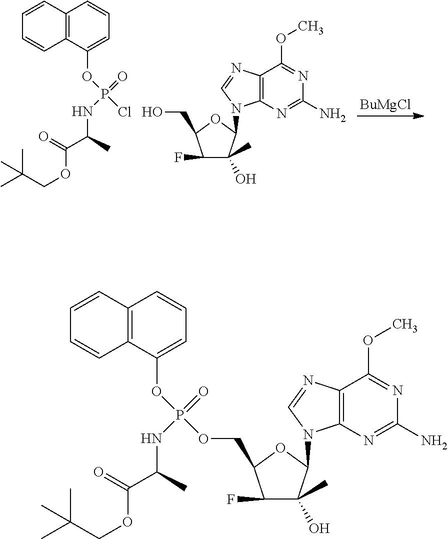

The nucleoside (2R,3S,4S,5R)-2-(2-amino-6-methoxy-9H-purin-9-yl)-4-fluoro-5-(hydroxymethyl)-3-methyltetrahydrofuran-3-ol (100 mg, 0.319 mmol) was dissolved in anhydrous THF (8 mL) and cooled to 0° C. followed by addition of t-Butyl magnesium chloride (0.638 mL, 0.638 mmol) and stirred for 15 min. To this reaction mixture was added with (S)-2-[Chloro-(naphthalen-1-yloxy)-phosphorylamino]-propionic acid 2,2-dimethyl propyl ester (306 mg, 0.798 mmol) and stirred at room temperature for 15 hrs. The reaction mixture was diluted with CH2Cl2 and washed with sa.aq.NH4Cl. The residue was purified by column chromatography to yield 61 mg of (2S)-Neopentyl 2-((((2R,3S,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate.

1H NMR (200 MHz, CD3OD): δ 8.18 (m, 1H), 7.9-7.3 (m, 7H), 6.02 (bs, 1H), 4.86 (m, 0.5H), 4.6 (m, 0.5H), 4.61 (m, 3H), 4.1 (m, 1H), 4.01 (s, 3H), 3.8-3.3.5 (m, 3H), 1.4 (m, 3H), 1.05 (s, 3H), 0.81 (2s, 9H)

19F NMR (188 MHz, CD3OD) δ −202.962, −203.116, −203.23, −203.39

31P NMR (80 MHz, CD3OD): δ 5.266, 5.153

### Example 30

### (2S)-Benzyl 2-((((2R,3S,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate

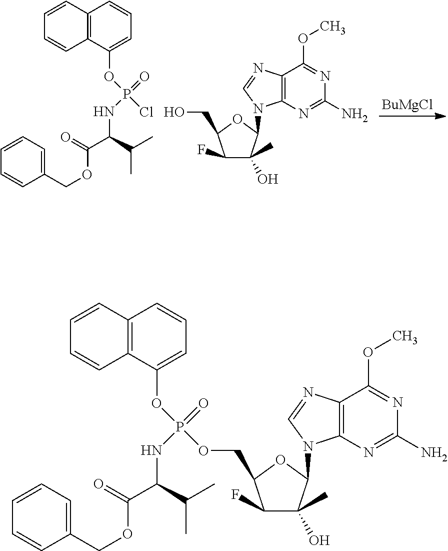

The nucleoside (2R,3S,4S,5R)-2-(2-amino-6-methoxy-9H-purin-9-yl)-4-fluoro-5-(hydroxymethyl)-3-methyltetrahydrofuran-3-ol (80 mg, 0.255 mmol) was dissolved in anhydrous THF (8 mL) and cooled to 0° C. followed by addition of t-Butyl magnesium chloride (0.511 mL, 0.511 mmol) and stirred for 15 min. To this reaction mixture was added with (S)-2-[Chloro-(naphthalen-1-yloxy)-phosphorylamino]-3-methyl butanoic acid benzyl ester (274 mg, 0.638 mmol) and stirred at room temperature for 15 hrs. The reaction mixture was diluted with CH2Cl2 and washed with sa.aq.NH4Cl. The residue was purified by column chromatography to yield 51 mg of (2S)-benzyl 2-((((2R,3S,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate

1H NMR (200 MHz, CD3OD): δ 8.2 (m, 1H), 7.82-7.6 (m, 3H), 7.5-7.3 (m, 5H), 7.2 (s, 5H), 6.05 (d, J=3 Hz, 1H), 5.05-4.9 (m, 2H), 4.82 (s, 4H), 4.75 (m, 1H), 4.6 (m, 3H), 4.01 (s, 3H), 3.82 (m, 1H), 2.01 (m, 1H), 1.08 (s, 3H), 0.81 (m, 6H)

19F NMR (188 MHz, CD3OD) δ 202.856, −202.95, −203.018, −203.132, −203.237, −203.302, −203.39

31P NMR (80 MHz, CD3OD): δ 6.066, 5.96

### Example 31

### (2S)-Neopentyl 2-((((2R,3R,4S,5R)-5-(6-amino-9H-purin-9-yl)-3-fluoro-4-hydroxy-4-methyltetrahydrofuran-2-yl)methoxy)(naphthalen-1-yloxy)phosphorylamino)propanoate

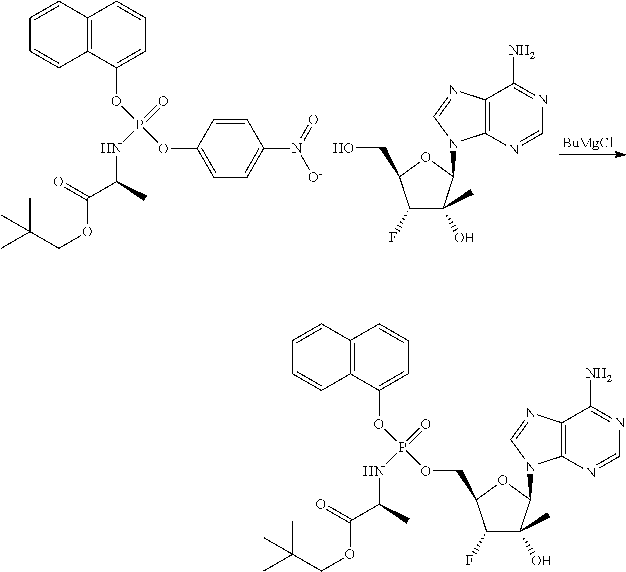

The nucleoside derivative (2R,3S,4R,5R)-2-(6-amino-9H-purin-9-yl)-4-fluoro-5-(hydroxymethyl)-3-methyltetrahydrofuran-3-ol, prepared as described in Example 8, ((44 mg, 0.155 mmol) was dissolved in DMF and cooled to 0° C. followed by addition of t-BuMgCl (1M in THF) (388 μL, 0.388 mmol) and stirred at 0° C. for 15 min. To this mixture was added (2S)-neopentyl 2-((naphthalen-1-yloxy)(4-nitrophenoxy)phosphorylamino)propanoate (189 mg, 0.388 mmol) and stirred at rt for 15 hrs. DMF was removed under reduced pressure. The resulting residue was dissolved in CH2Cl2 and washed with sat.aq.NH4Cl, dried with anhyd.Na2SO4, filtered and concentrated. This crude mixture was purified with column chromatography with stepwise gradient of MeOH (2-6%) in CH2Cl2 to afford 71 mg of (2S)-neopentyl 2-((((2R,3R,4 S,5R)-5-(6-amino-9H-purin-9-yl)-3-fluoro-4-hydroxy-4-methyltetrahydrofuran-2-yl)methoxy)(naphthalen-1-yloxy)phosphorylamino)propanoate.

1H NMR (200 MHz, CD3OD): δ 8.15 (m, 3H), 7.81 (m, 1H), 7.65 (m, 1H), 7.6-7.3 (m, 4H), 6.06 (d, J=3.2 Hz, 1H), 5.3 (d, J=5.4 Hz, 0.5H), 5.01 (d, J=5.4 Hz, 0.5H), 4.6 (m, 3H), 4.1 (m, 1H), 3.8-3.6 (m, 2H), 1.3 (d, J=6 Hz, 3H), 1.01 (s, 3H), 0.83 (s, 9H)

19F NMR (188 MHz, CD3OD): δ− 217.1 (2), −217.8 (2)

31P NMR (80 MHz, CD3OD): δ 5.266, 5.278

### Example 32

### (2S)-Neopentyl 2-((((2R,3R,4S,5R)-5-(4-amino-2-oxopyrimidin-1(2H)-yl)-3-fluoro-4-hydroxy-4-methyltetrahydrofuran-2-yl)methoxy)(naphthalen-1-yloxy)phosphorylamino)propanoate

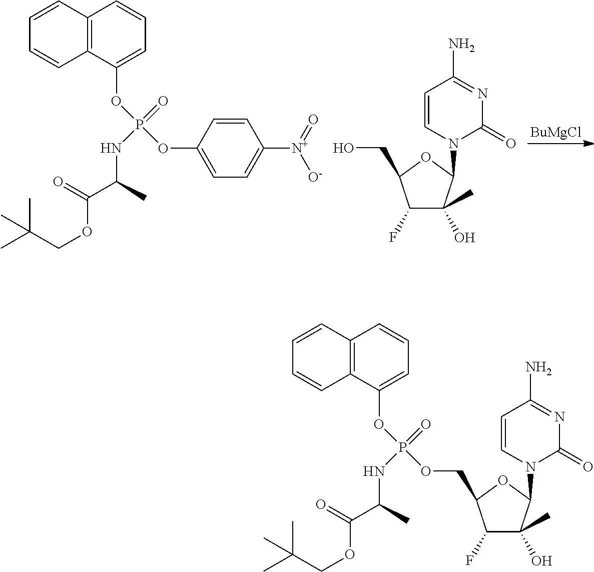

4-Amino-1-((2R,3S,4R,5R)-4-fluoro-3-hydroxy-5-(hydroxymethyl)-3-methyltetrahydrofuran-2-yl)pyrimidin-2(1H)-one (Example 9) (34 mg, 0.132 mmol) was dissolved in DMF and cooled to 0° C. followed by addition of t-BuMgCl (1M in THF) (328 μL, 0.328 mmol) and stirred at 0° C. for 15 min. To this mixture was added (2S)-neopentyl 2-((naphthalen-1-yloxy)(4-nitrophenoxy)phosphorylamino)propanoate (160 mg, 0.328 mmol) and stirred at rt for 15 hrs. DMF was removed under reduced pressure. The resulting residue was dissolved in CH2Cl2 and washed with sat.aq.NH4Cl, dried with anhyd.Na2SO4, filtered and concentrated. This crude mixture was purified with column chromatography with stepwise gradient of MeOH (2-6%) in CH2Cl2 to afford 61 mg of (2S)-Neopentyl 2-((((2R,3R,4S,5R)-5-(4-amino-2-oxopyrimidin-1(2H)-yl)-3-fluoro-4-hydroxy-4-methyltetrahydrofuran-2-yl)methoxy)(naphthalen-1-yloxy)phosphorylamino)propanoate.

0.1H NMR (200 MHz, CD3OD): δ 8.2 (m, 1H), 7.88 (m, 1H), 7.7 (m, 1H), 7.6-7.3 (m, 6H), 5.97 (d, J=3 Hz, 1H), 5.7 (m, 1H), 4.8-4.3 (m, 4H), 4.02 (m, 1H), 3.9-3.5 (m, 3H), 1.41 (d, J=6 Hz, 3H), 1.05 (s, 3H), 0.91 (s, 9H)

19F NMR (188 MHz, CD3OD): δ −214.38, −214.65, −215.38, −215.642

31P NMR (80 MHz, CD3OD): δ 5.296, 5.228

### Example 33

### (2 S)-Neopentyl 2-((((2R,3R,4S,5R)-5-(2,4-dioxo-3,4-dihydropyrimidin-1(2H)-yl)-3-fluoro-4-hydroxy-4-methyltetrahydrofuran-2-yl)methoxy)(naphthalen-1-yloxy)phosphorylamino)propanoate

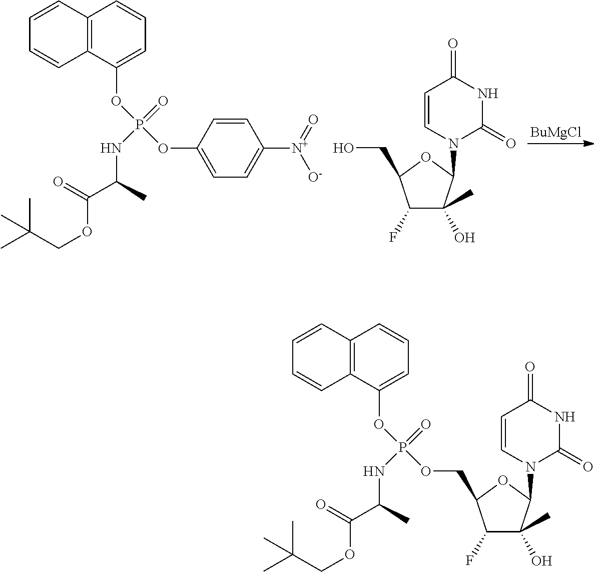

1-((2R,3S,4R,5R)-4-Fluoro-3-hydroxy-5-(hydroxymethyl)-3-methyltetrahydrofuran-2-yl)pyrimidine-2,4(1H,3H)-dione, prepared in Example 10, (50 mg, 0.192 mmol) was dissolved in DMSO followed by addition of t-BuMgCl (1M in THF) (480 μL, 0.480 mmol) and stirred at room temperature for 15 min. To this mixture was added (2S)-neopentyl 2-((naphthalen-1-yloxy)(4-nitrophenoxy)phosphorylamino)propanoate (233.7 mg, 0.480 mmol) and stirred at rt for 15 hrs. DMSO was removed under reduced pressure. The resulting residue was dissolved in CH2Cl2 and washed with sat.aq.NH4Cl, dried with anhyd.Na2SO4, filtered and concentrated. This crude mixture was purified with column chromatography with stepwise gradient of MeOH (2-5%) in CH2Cl2 to afford 102 mg of compound (2 S)-neopentyl 2-((((2R,3R,4S,5R)-5-(2,4-dioxo-3,4-dihydropyrimidin-1(2H)-yl)-3-fluoro-4-hydroxy-4-methyltetrahydrofuran-2-yl)methoxy)(naphthalen-1-yloxy)phosphorylamino)propanoate.

1H NMR (200 MHz, CD3OD): δ 8.2 (m, 1H), 7.9 (m, 1H), 7.7 (m, 1H), 7.6-7.35 (m, 6H), 5.88 (d, J=3 Hz, 1H), 5.4 (m, 1H), 4.83 (bs, 3H), 4.8-4.3 (m, 5H), 4.1 (m, 1H), 3.8 (m, 2H), 1.4 (dd, J=8 Hz, J=3 Hz, 3H), 1.1 (d, J=7 Hz, 3H), 0.85 (s, 9H)

19F NMR (188 MHz, CD3OD): δ− 214.07, −214.14, −214.35, −214.43, −215.19, −215.27, −215.48, −215.55

31P NMR (80 MHz, CD3OD): δ 5.296, 5.213

### Example 34

### ((2R,4S,5R)-5-(2-Amino-6-oxo-1H-purin-9(6H)-yl)-3-fluoro-4-hydroxy-4-methyltetrahydrofuran-2-yl)methyl tetrahydrogen triphosphate

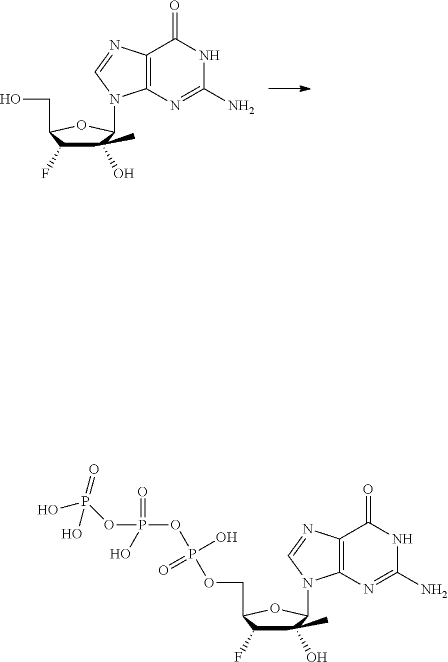 [SMILES: C[C@@]1(O)[C@H](F)[C@@H](CO)O[C@H]1n1cnc2c(=O)[nH]c(N)nc21 ; C[C@@]1(O)[C@H](F)[C@@H](COP(=O)(O)OP(=O)(O)OP(=O)(O)O)O[C@H]1n1cnc2c(=O)[nH]c(N)nc21]

The nucleoside 2-amino-9-((2R,3S,5R)-4-fluoro-3-hydroxy-5-(hydroxymethyl)-3-methyltetrahydrofuran-2-yl)-1H-purin-6(9H)-one (50 mg), prepared as described in Example 3, and N,N,N′,N′-tetramethyl-1,8-naphthalenediamine (proton sponge, 72 mg) were taken in anhydrous PO(OMe)3 (2.5 mL) under an inert atmosphere. After stirring at room temperature for 5-10 minutes, the solution becomes clear. The reaction mixture was then cooled to 0-5° C. (ice-bath), and after 15 minutes POCl3 (32 μL) was added drop wise. Stirring was continued at 0-5° C. for 5 h, TLC (30% MeOH in CHCl3) at this time showed a more polar spot compared to the starting nucleoside. Mixture of pyrophosphate in DMF (0.84 mL, 1M solution) and TBA (0.16 mL) were added. Stirring was continued at 0-5° C. for 2.5 h followed by the addition of aqueous ammonium bicarbonate (AB) (4.1 mL, 1M solution). The reaction mixture was set to warm to room temperature over an hour. After dilution with water (˜5 mL) and extraction with MTBE (20 mL×3), the aqueous layer was concentrated (not heating above 45° C.) or lyophilized. The residue was loaded onto Sephadex column eluting with gradient of 0.2 M AB to 0.4 M AB. The fractions containing the desired product are pooled together and lyophilized. The desired product (20 mg) was obtained as an off-white solid. Spectral data (1H, 31P NMR and LC-MS) were consistent with the proposed structure.

1H NMR (300 MHz, D2O): δ 8.05 (s, 1H), 5.95 (br s, 1H), 5.2 (d, 1H), 5.0 (d, 1H), 4.5-4.4 (m, 1H), 4.35-4.25 (m, 2H), 1.05 (s, 3H)

31P NMR (120 MHz, D2O): δ −9.6 (d), −10.05 (d), −22.0 (t).

Further to the above Examples, representative compounds, prepared according to the examples were tested for potency in an HCV replicon assay (Genotype 1b) for activity against the virus (EC50) and toxicity to the cells (CC50). These results are set forth below.

Huh7 Replicon Cell Lines and Cell Culture Conditions: A luciferase-reporter genotype 1b subgenomic replicon cell line, and a genotype 1a full-length replicon cell line were obtained from Apath, LLC, Brooklyn, N.Y.: All cell lines were passaged twice a week by splitting 4 or 6 fold. Cells were maintained in DMEM-high glucose medium (HyClone, Logan, Utah) supplemented with 9% FBS (HyClone), 2 mM glutamine (Invitrogen, Carlsbad, Calif.), 100 U/mL PenStrep (Invitrogen). Media also contained 0.25 mg/mL of the antibiotic G-418 to maintain stable expression of the replicon (Invitrogen). Incubation was performed at 37° C. in 5% CO2 atmosphere. Replicon cell lines were used until they accumulated 15-to-18 passages, after which cells were restarted from the frozen stock. Seeding cell counts were routinely determined using an automatic Cedex HiRes cell counter (Flownomics Analytical Instruments, Madison, Wis.) or manually using INCYTO C-Chip™ Disposable Hemacytometers (Fisher Scientific, Pittsburg, Pa.).

The Anti-HCV Assays were Done Accordingly:

Luciferase Genotype 1b Replicon Potency Assay. Replicon cells were seeded into white 96-well plates (Nunc/VWR) at a density of 2×104 cells/well in medium without G-418. A Stacker Multidrop Liquid Dispenser (MTX Lab Systems, Vienna, Va.) was employed to ensure uniform and fast cell seeding into multiple plates. 18-24 h after cell plating, inhibitors were added and cells were incubated for additional 24, 48, or 72 h (as indicated). Compounds were tested in triplicates and quadruplicates at 3× or 4× serial dilutions over a range of 0.0001-to-10 μM concentrations. HCV replication was monitored by Renilla luciferase reporter activity assay using Renilla luciferase reporter (Promega, Madison, Wis.) and a Veritas Luminometer (Turner Biosystems, Sunnyvale, Calif.). 50% and 90% inhibitory concentration (IC50 and IC90) values were calculated as the concentration of compound that results correspondingly in 50% and 90% decreases in the reporter expression as compared to untreated cells. The values were determined by non-linear regression (four-parameter sigmoidal curve fitting) analysis.

### The Cell Cytotoxicity Assay Data was Obtained as Described Below:

Cytotoxicity Assay. Cells were seeded into 96-well plates at a density of 2×104 cells per well. 24 h after cell plating, 11 serial 2× compound dilutions, starting with 100 □M, were applied to the testing plates (3 repeats per compound dilution). Each testing plate was run with a “no-compound” control. Incubation with compounds was continued at 37° C. in a CO2 incubator for 72 h. To determine cell viability, the CellTiter-Glo® assay (Promega, Madison, Wis.) was performed according to the manufacturer's protocol. The compound concentration resulting in 50% luminescent signal was reported as the CC50 concentration.

The results of the assay in terms of IC50 (μM) and CC50 (μM) are given in Table 1 below:

TABLE 1

| | | IC50 | CC50 |
|---|---|---|---|
| Example # | Structure | μM | μM |
| 1 | 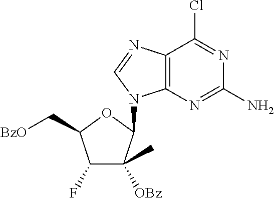 | — | — |
| 2 | 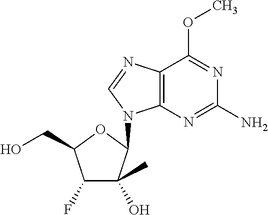 [SMILES: COc1nc(N)nc2c1ncn2[C@@H]1O[C@H](CO)[C@@H](F)[C@@]1(C)O] | >100 | >100 |
| 3 | 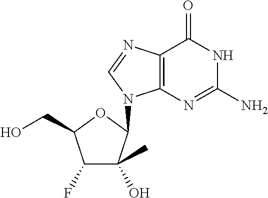 [SMILES: C[C@@]1(O)[C@H](F)[C@@H](CO)O[C@H]1n1cnc2c(=O)[nH]c(N)nc21] | >100 | >100 |
| 4 | 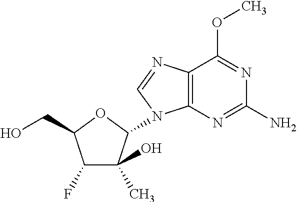 [SMILES: COc1nc(N)nc2c1ncn2[C@H]1O[C@H](CO)[C@@H](F)[C@]1(C)O] | >100 | >100 |
| 5 | 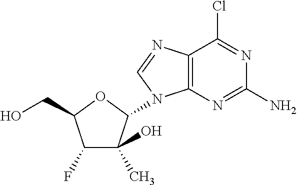 [SMILES: C[C@]1(O)[C@H](F)[C@@H](CO)O[C@@H]1n1cnc2c(Cl)nc(N)nc21] | >100 | >100 |
| 6 | 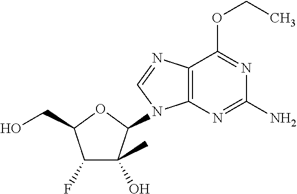 [SMILES: CCOc1nc(N)nc2c1ncn2[C@@H]1O[C@H](CO)[C@@H](F)[C@@]1(C)O] | >100 | >100 |
| 7 | 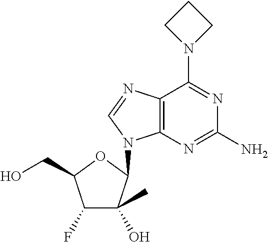 [SMILES: C[C@@]1(O)[C@H](F)[C@@H](CO)O[C@H]1n1cnc2c(N3CCC3)nc(N)nc21] | 24 >40 | >100 |
| 8 | 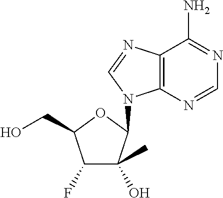 [SMILES: C[C@@]1(O)[C@H](F)[C@@H](CO)O[C@H]1n1cnc2c(N)ncnc21] | >100 | >100 |
| 9 | 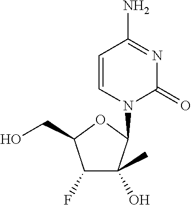 [SMILES: C[C@@]1(O)[C@H](F)[C@@H](CO)O[C@H]1n1ccc(N)nc1=O] | >100 | >100 |
| 10 | 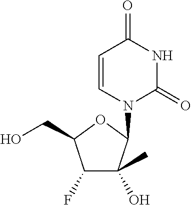 [SMILES: C[C@@]1(O)[C@H](F)[C@@H](CO)O[C@H]1n1ccc(=O)[nH]c1=O] | 23 | >100 |
| 11 | 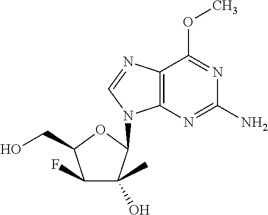 [SMILES: COc1nc(N)nc2c1ncn2[C@@H]1O[C@H](CO)[C@H](F)[C@@]1(C)O] | >100 | >100 |
| 12 | 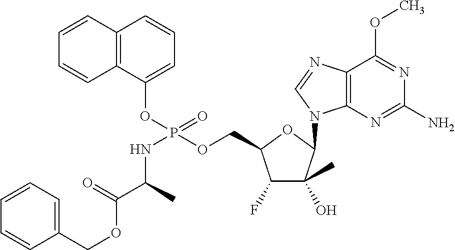 [SMILES: COc1nc(N)nc2c1ncn2[C@@H]1O[C@H](COP(=O)(N[C@@H](C)C(=O)OCc2ccccc2)Oc2cccc3ccccc23)[C@@H](F)[C@@]1(C)O] | 0.77 | 48 |
| 13 | 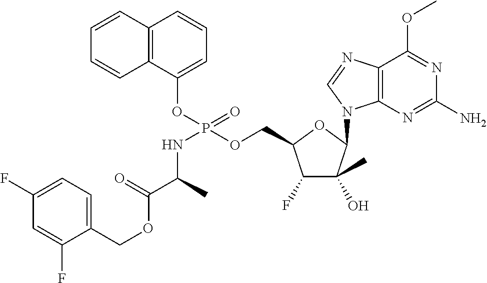 [SMILES: COc1nc(N)nc2c1ncn2[C@@H]1O[C@H](COP(=O)(N[C@@H](C)C(=O)OCc2ccc(F)cc2F)Oc2cccc3ccccc23)[C@@H](F)[C@@]1(C)O] | 1.0 | 68 |
| 14 | 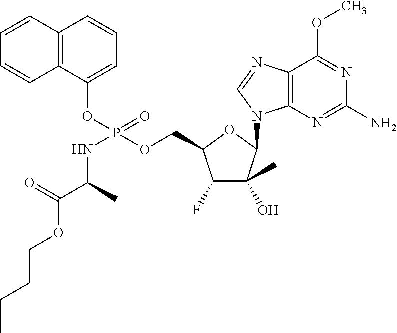 [SMILES: CCCCOC(=O)[C@H](C)NP(=O)(OC[C@H]1O[C@@H](n2cnc3c(OC)nc(N)nc32)[C@](C)(O)[C@@H]1F)Oc1cccc2ccccc12] | 0.33 | 40 |
| 15 | 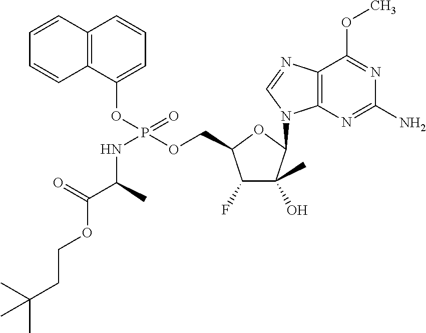 [SMILES: COc1nc(N)nc2c1ncn2[C@@H]1O[C@H](COP(=O)(N[C@@H](C)C(=O)OCCC(C)(C)C)Oc2cccc3ccccc23)[C@@H](F)[C@@]1(C)O] | 0.5 | 21 |
| 16 | 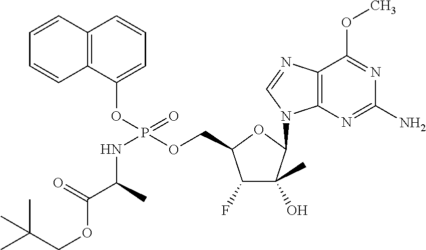 [SMILES: COc1nc(N)nc2c1ncn2[C@@H]1O[C@H](COP(=O)(N[C@@H](C)C(=O)OCC(C)(C)C)Oc2cccc3ccccc23)[C@@H](F)[C@@]1(C)O] | 0.37 | 29 |
| 17 | 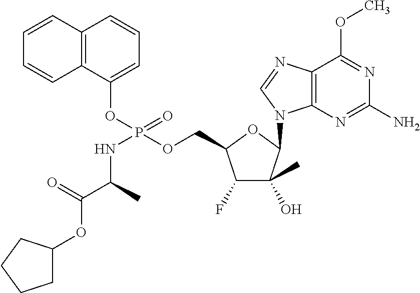 [SMILES: COc1nc(N)nc2c1ncn2[C@@H]1O[C@H](COP(=O)(N[C@@H](C)C(=O)OC2CCCC2)Oc2cccc3ccccc23)[C@@H](F)[C@@]1(C)O] | 0.45 | 40 |
| 18 | 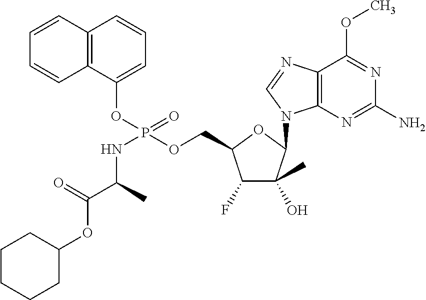 [SMILES: COc1nc(N)nc2c1ncn2[C@@H]1O[C@H](COP(=O)(N[C@@H](C)C(=O)OC2CCCCC2)Oc2cccc3ccccc23)[C@@H](F)[C@@]1(C)O] | 1.3 | 30 |
| 19 | 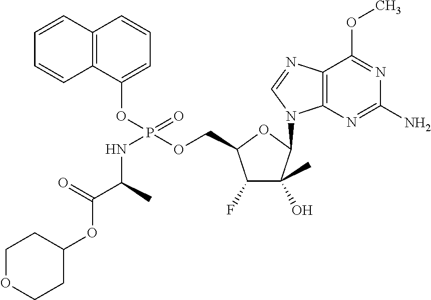 [SMILES: COc1nc(N)nc2c1ncn2[C@@H]1O[C@H](COP(=O)(N[C@@H](C)C(=O)OC2CCOCC2)Oc2cccc3ccccc23)[C@@H](F)[C@@]1(C)O] | 1.4 | 77 |
| 20 | 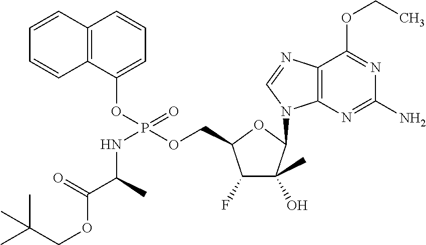 [SMILES: CCOc1nc(N)nc2c1ncn2[C@@H]1O[C@H](COP(=O)(N[C@@H](C)C(=O)OCC(C)(C)C)Oc2cccc3ccccc23)[C@@H](F)[C@@]1(C)O] | 0.62 | 23 |
| 21 | 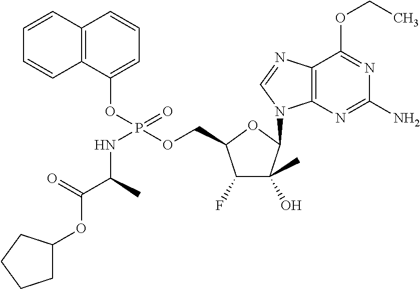 [SMILES: CCOc1nc(N)nc2c1ncn2[C@@H]1O[C@H](COP(=O)(N[C@@H](C)C(=O)OC2CCCC2)Oc2cccc3ccccc23)[C@@H](F)[C@@]1(C)O] | 0.40 | 28 |
| 22 | 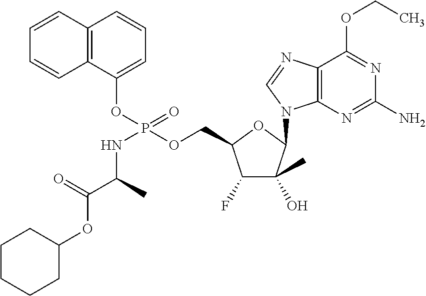 [SMILES: CCOc1nc(N)nc2c1ncn2[C@@H]1O[C@H](COP(=O)(N[C@@H](C)C(=O)OC2CCCCC2)Oc2cccc3ccccc23)[C@@H](F)[C@@]1(C)O] | 0.73 | 16 |
| 23 | 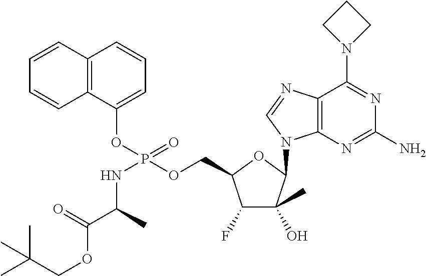 [SMILES: C[C@H](NP(=O)(OC[C@H]1O[C@@H](n2cnc3c(N4CCC4)nc(N)nc32)[C@](C)(O)[C@@H]1F)Oc1cccc2ccccc12)C(=O)OCC(C)(C)C] | 0.90 | 49 |
| 24 | 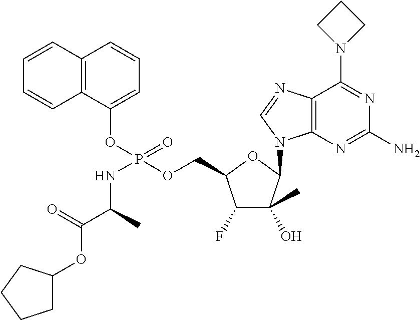 [SMILES: C[C@H](NP(=O)(OC[C@H]1O[C@@H](n2cnc3c(N4CCC4)nc(N)nc32)[C@](C)(O)[C@@H]1F)Oc1cccc2ccccc12)C(=O)OC1CCCC1] | 0.90 | 50 |
| 25 | 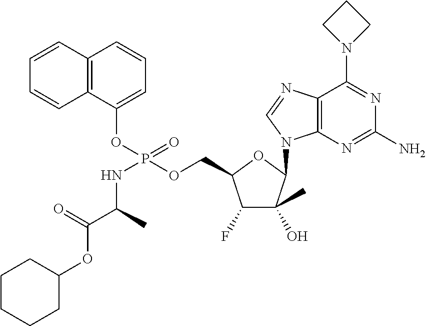 [SMILES: C[C@H](NP(=O)(OC[C@H]1O[C@@H](n2cnc3c(N4CCC4)nc(N)nc32)[C@](C)(O)[C@@H]1F)Oc1cccc2ccccc12)C(=O)OC1CCCCC1] | 0.95 | 33 |
| 26 | 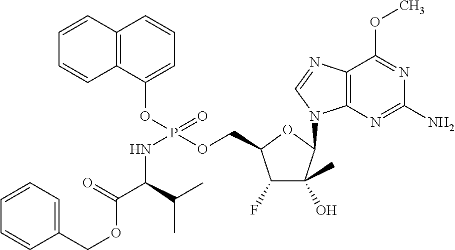 [SMILES: COc1nc(N)nc2c1ncn2[C@@H]1O[C@H](COP(=O)(N[C@H](C(=O)OCc2ccccc2)C(C)C)Oc2cccc3ccccc23)[C@@H](F)[C@@]1(C)O] | 4.0 | 14 |
| 27 | 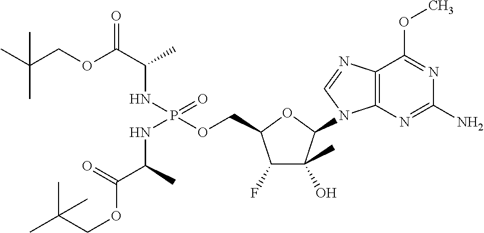 [SMILES: COc1nc(N)nc2c1ncn2[C@@H]1O[C@H](COP(=O)(N[C@@H](C)C(=O)OCC(C)(C)C)N[C@@H](C)C(=O)OCC(C)(C)C)[C@@H](F)[C@@]1(C)O] | 2.1 | >100 |
| 28 | 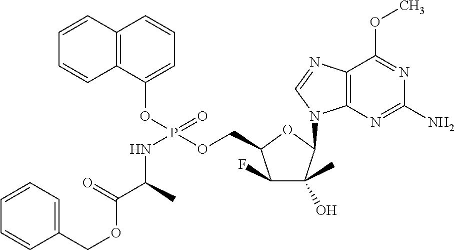 [SMILES: COc1nc(N)nc2c1ncn2[C@@H]1O[C@H](COP(=O)(N[C@@H](C)C(=O)OCc2ccccc2)Oc2cccc3ccccc23)[C@H](F)[C@@]1(C)O] | >40 | 92 |
| 29 | 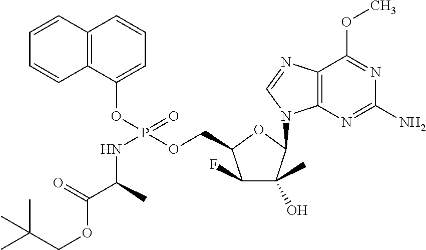 [SMILES: COc1nc(N)nc2c1ncn2[C@@H]1O[C@H](COP(=O)(N[C@@H](C)C(=O)OCC(C)(C)C)Oc2cccc3ccccc23)[C@H](F)[C@@]1(C)O] | 26 | 40 |
| 30 | 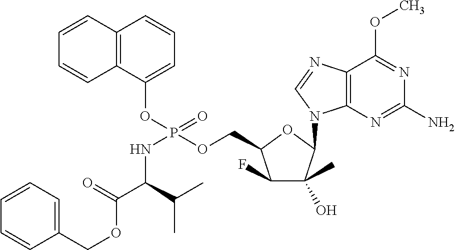 [SMILES: COc1nc(N)nc2c1ncn2[C@@H]1O[C@H](COP(=O)(N[C@H](C(=O)OCc2ccccc2)C(C)C)Oc2cccc3ccccc23)[C@H](F)[C@@]1(C)O] | 11 | 19 |
| 31 | 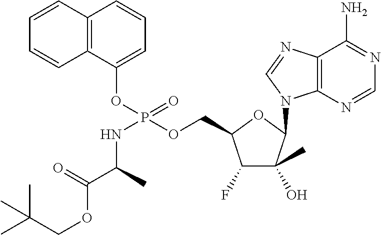 [SMILES: C[C@H](NP(=O)(OC[C@H]1O[C@@H](n2cnc3c(N)ncnc32)[C@](C)(O)[C@@H]1F)Oc1cccc2ccccc12)C(=O)OCC(C)(C)C] | 11 | 31 |
| 32 | 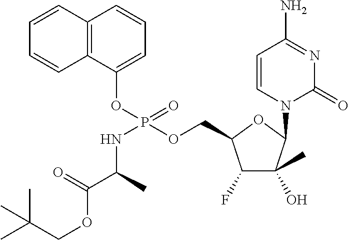 [SMILES: C[C@H](NP(=O)(OC[C@H]1O[C@@H](n2ccc(N)nc2=O)[C@](C)(O)[C@@H]1F)Oc1cccc2ccccc12)C(=O)OCC(C)(C)C] | >40 | >100 |
| 33 | 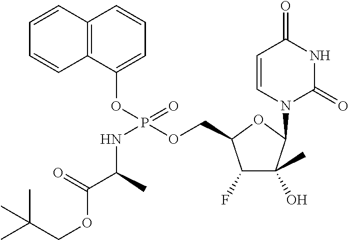 [SMILES: C[C@H](NP(=O)(OC[C@H]1O[C@@H](n2ccc(=O)[nH]c2=O)[C@](C)(O)[C@@H]1F)Oc1cccc2ccccc12)C(=O)OCC(C)(C)C] | >40 | 75 |
| 34 | 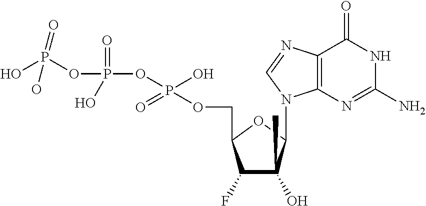 | — | — |

### Additional Examples

In addition, the following prodrugs of 3′ Fluoro are provided in accordance with the invention:

TABLE 2

| Prodrug | |
|---|---|
| Example# | Structure |
| 1 | 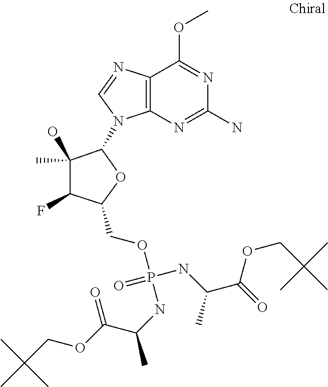 [SMILES: COc1nc(N)nc2c1ncn2[C@@H]1O[C@H](COP(=O)(N[C@@H](C)C(=O)OCC(C)(C)C)N[C@@H](C)C(=O)OCC(C)(C)C)[C@@H](F)[C@@]1(C)O] |
| 2 | 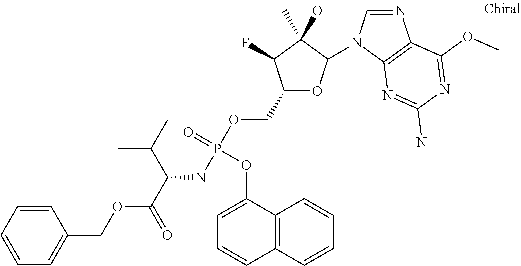 [SMILES: COc1nc(N)nc2c1ncn2C1O[C@H](COP(=O)(N[C@H](C(=O)OCc2ccccc2)C(C)C)Oc2cccc3ccccc23)[C@@H](F)[C@@]1(C)O] |
| 3 | 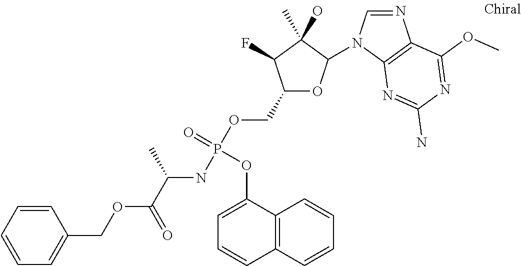 [SMILES: COc1nc(N)nc2c1ncn2C1O[C@H](COP(=O)(N[C@@H](C)C(=O)OCc2ccccc2)Oc2cccc3ccccc23)[C@@H](F)[C@@]1(C)O] |
| 4 | 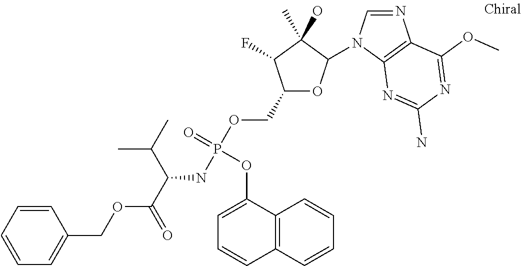 [SMILES: COc1nc(N)nc2c1ncn2C1O[C@H](COP(=O)(N[C@H](C(=O)OCc2ccccc2)C(C)C)Oc2cccc3ccccc23)[C@H](F)[C@@]1(C)O] |
| 5 | 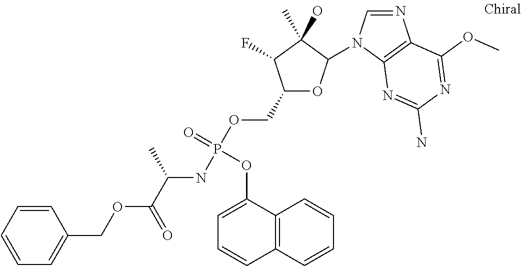 [SMILES: COc1nc(N)nc2c1ncn2C1O[C@H](COP(=O)(N[C@@H](C)C(=O)OCc2ccccc2)Oc2cccc3ccccc23)[C@H](F)[C@@]1(C)O] |
| 6 | 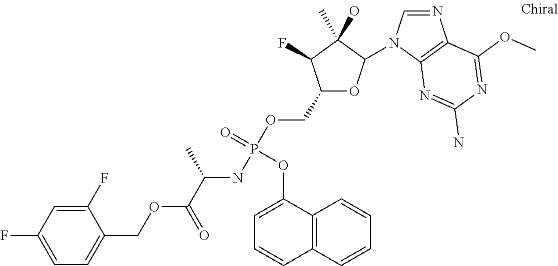 [SMILES: COc1nc(N)nc2c1ncn2C1O[C@H](COP(=O)(N[C@@H](C)C(=O)OCc2ccc(F)cc2F)Oc2cccc3ccccc23)[C@@H](F)[C@@]1(C)O] |
| 7 | 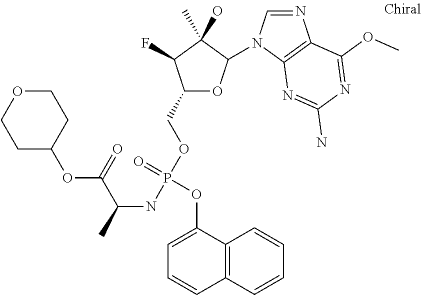 [SMILES: COc1nc(N)nc2c1ncn2C1O[C@H](COP(=O)(N[C@@H](C)C(=O)OC2CCOCC2)Oc2cccc3ccccc23)[C@@H](F)[C@@]1(C)O] |
| 8 | 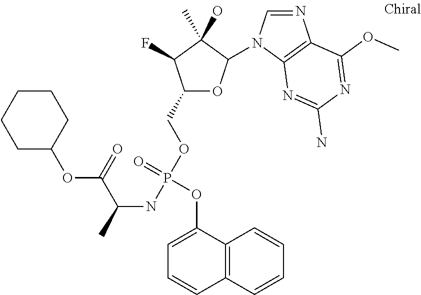 [SMILES: COc1nc(N)nc2c1ncn2C1O[C@H](COP(=O)(N[C@@H](C)C(=O)OC2CCCCC2)Oc2cccc3ccccc23)[C@@H](F)[C@@]1(C)O] |
| 9 | 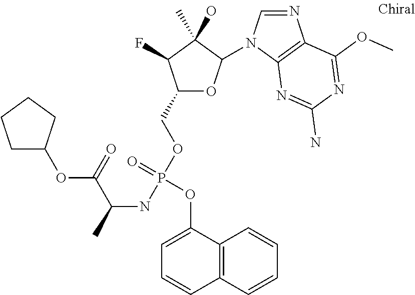 [SMILES: COc1nc(N)nc2c1ncn2C1O[C@H](COP(=O)(N[C@@H](C)C(=O)OC2CCCC2)Oc2cccc3ccccc23)[C@@H](F)[C@@]1(C)O] |
| 10 | 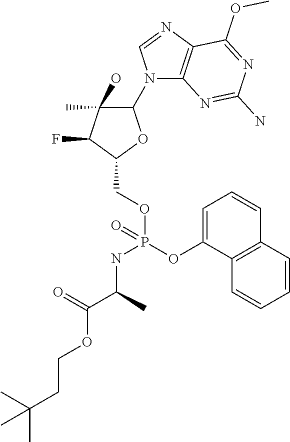 [SMILES: COc1nc(N)nc2c1ncn2C1O[C@H](COP(=O)(N[C@@H](C)C(=O)OCCC(C)(C)C)Oc2cccc3ccccc23)[C@@H](F)[C@@]1(C)O] |
| 11 | 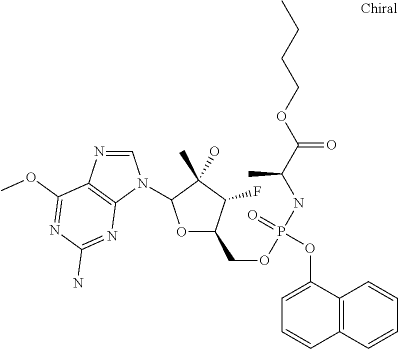 [SMILES: CCCCOC(=O)[C@H](C)NP(=O)(OC[C@H]1OC(n2cnc3c(OC)nc(N)nc32)[C@](C)(O)[C@@H]1F)Oc1cccc2ccccc12] |
| 12 | 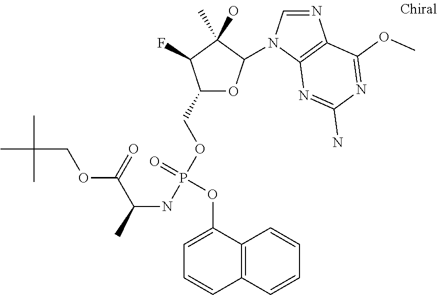 [SMILES: COc1nc(N)nc2c1ncn2C1O[C@H](COP(=O)(N[C@@H](C)C(=O)OCC(C)(C)C)Oc2cccc3ccccc23)[C@@H](F)[C@@]1(C)O] |
| 13 | 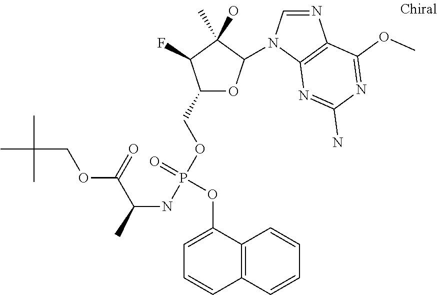 [SMILES: COc1nc(N)nc2c1ncn2C1O[C@H](COP(=O)(N[C@@H](C)C(=O)OCC(C)(C)C)Oc2cccc3ccccc23)[C@@H](F)[C@@]1(C)O] |
| 14 | 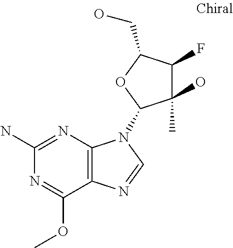 [SMILES: COc1nc(N)nc2c1ncn2[C@@H]1O[C@H](CO)[C@@H](F)[C@@]1(C)O] |
| 15 | 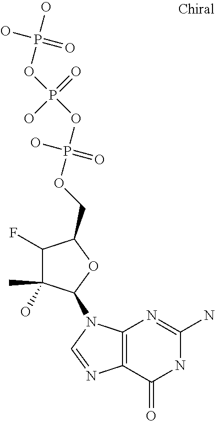 [SMILES: C[C@@]1(O)C(F)[C@@H](COP(=O)(O)OP(=O)(O)OP(=O)(O)O)O[C@H]1n1cnc2c(=O)[nH]c(N)nc21] |

In addition, the following alternative bases are provided in accordance with the invention:

TABLE 3

| Alternative | |
|---|---|
| Bases | |
| Example# | Structure |
| 1 | 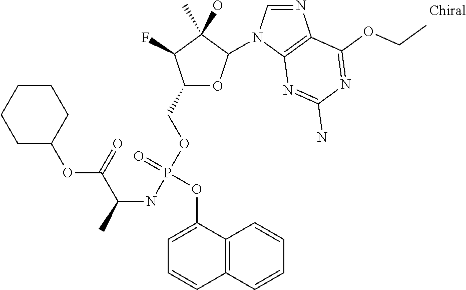 [SMILES: CCOc1nc(N)nc2c1ncn2C1O[C@H](COP(=O)(N[C@@H](C)C(=O)OC2CCCCC2)Oc2cccc3ccccc23)[C@@H](F)[C@@]1(C)O] |
| 2 | 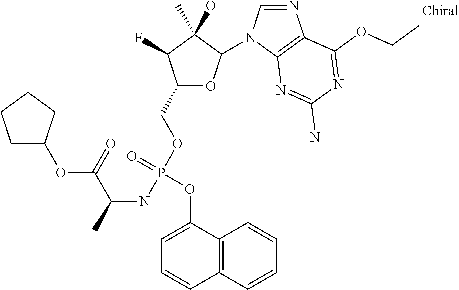 [SMILES: CCOc1nc(N)nc2c1ncn2C1O[C@H](COP(=O)(N[C@@H](C)C(=O)OC2CCCC2)Oc2cccc3ccccc23)[C@@H](F)[C@@]1(C)O] |
| 3 | 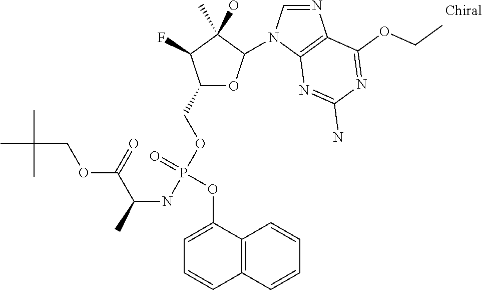 [SMILES: CCOc1nc(N)nc2c1ncn2C1O[C@H](COP(=O)(N[C@@H](C)C(=O)OCC(C)(C)C)Oc2cccc3ccccc23)[C@@H](F)[C@@]1(C)O] |
| 4 | 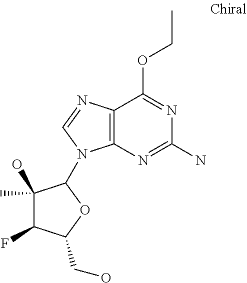 [SMILES: CCOc1nc(N)nc2c1ncn2C1O[C@H](CO)[C@@H](F)[C@@]1(C)O] |
| 5 | 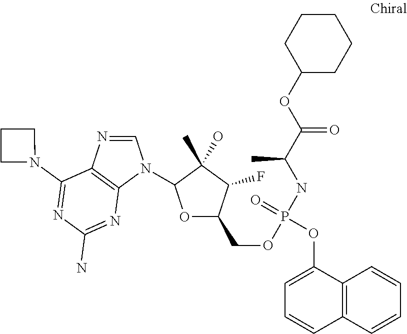 [SMILES: C[C@H](NP(=O)(OC[C@H]1OC(n2cnc3c(N4CCC4)nc(N)nc32)[C@](C)(O)[C@@H]1F)Oc1cccc2ccccc12)C(=O)OC1CCCCC1] |
| 6 | 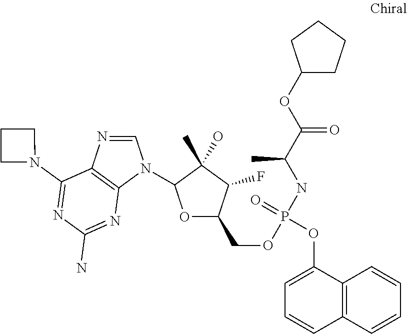 [SMILES: C[C@H](NP(=O)(OC[C@H]1OC(n2cnc3c(N4CCC4)nc(N)nc32)[C@](C)(O)[C@@H]1F)Oc1cccc2ccccc12)C(=O)OC1CCCC1] |
| 7 | 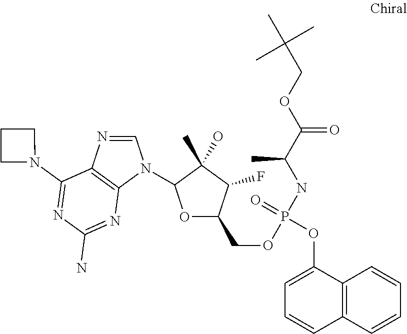 [SMILES: C[C@H](NP(=O)(OC[C@H]1OC(n2cnc3c(N4CCC4)nc(N)nc32)[C@](C)(O)[C@@H]1F)Oc1cccc2ccccc12)C(=O)OCC(C)(C)C] |
| 8 | 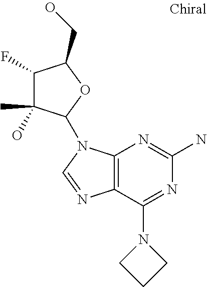 [SMILES: C[C@]1(O)C(n2cnc3c(N4CCC4)nc(N)nc32)O[C@H](CO)[C@H]1F] |
| 9 | 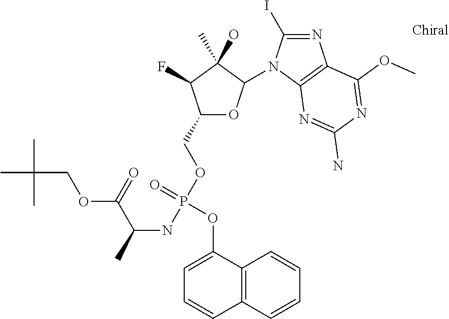 [SMILES: COc1nc(N)nc2c1nc(I)n2C1O[C@H](COP(=O)(N[C@@H](C)C(=O)OCC(C)(C)C)Oc2cccc3ccccc23)[C@@H](F)[C@@]1(C)O] |
| 10 | 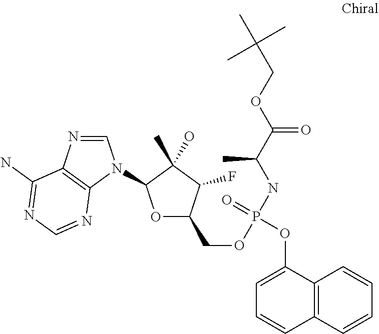 [SMILES: C[C@H](NP(=O)(OC[C@H]1O[C@@H](n2cnc3c(N)ncnc32)[C@](C)(O)[C@@H]1F)Oc1cccc2ccccc12)C(=O)OCC(C)(C)C] |
| 11 | 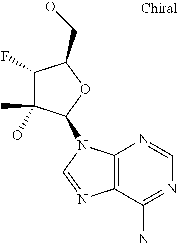 [SMILES: C[C@@]1(O)[C@H](F)[C@@H](CO)O[C@H]1n1cnc2c(N)ncnc21] |
| 12 | 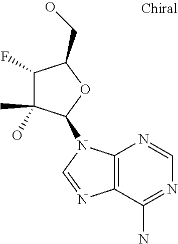 [SMILES: C[C@@]1(O)[C@H](F)[C@@H](CO)O[C@H]1n1cnc2c(N)ncnc21] |
| 13 | 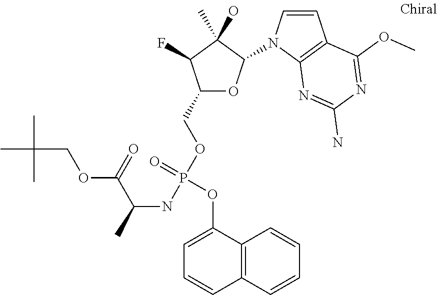 [SMILES: COc1nc(N)nc2c1ccn2[C@@H]1O[C@H](COP(=O)(N[C@@H](C)C(=O)OCC(C)(C)C)Oc2cccc3ccccc23)[C@@H](F)[C@@]1(C)O] |
| 14 | 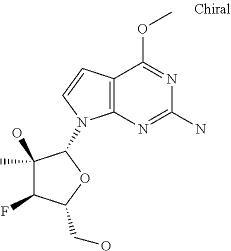 [SMILES: COc1nc(N)nc2c1ccn2[C@@H]1O[C@H](CO)[C@@H](F)[C@@]1(C)O] |
| 15 | 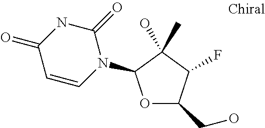 [SMILES: C[C@@]1(O)[C@H](F)[C@@H](CO)O[C@H]1n1ccc(=O)[nH]c1=O] |
| 16 | 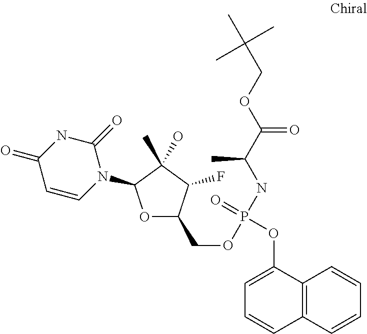 [SMILES: C[C@H](NP(=O)(OC[C@H]1O[C@@H](n2ccc(=O)[nH]c2=O)[C@](C)(O)[C@@H]1F)Oc1cccc2ccccc12)C(=O)OCC(C)(C)C] |
| 17 | 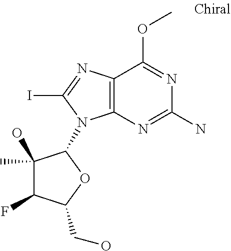 [SMILES: COc1nc(N)nc2c1nc(I)n2[C@@H]1O[C@H](CO)[C@@H](F)[C@@]1(C)O] |
| 18 | 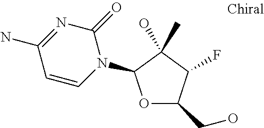 [SMILES: C[C@@]1(O)[C@H](F)[C@@H](CO)O[C@H]1n1ccc(N)nc1=O] |
| 19 | 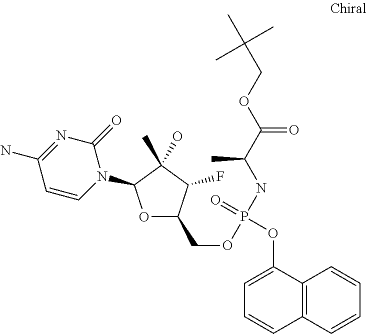 [SMILES: C[C@H](NP(=O)(OC[C@H]1O[C@@H](n2ccc(N)nc2=O)[C@](C)(O)[C@@H]1F)Oc1cccc2ccccc12)C(=O)OCC(C)(C)C] |
| 20 | 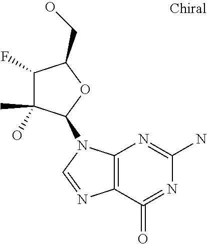 |

Finally, the following alternative bases are provided in accordance with the invention:

TABLE 4

| Alternate | |
|---|---|
| sugars | |
| Example# | Structure |
| 1 | 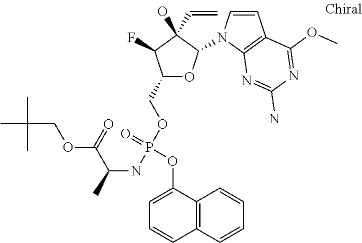 [SMILES: C=C[C@@]1(O)[C@H](F)[C@@H](COP(=O)(N[C@@H](C)C(=O)OCC(C)(C)C)Oc2cccc3ccccc23)O[C@H]1n1ccc2c(OC)nc(N)nc21] |
| 2 | 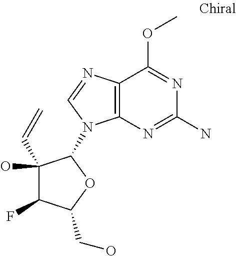 [SMILES: C=C[C@@]1(O)[C@H](F)[C@@H](CO)O[C@H]1n1cnc2c(OC)nc(N)nc21] |
| 3 | 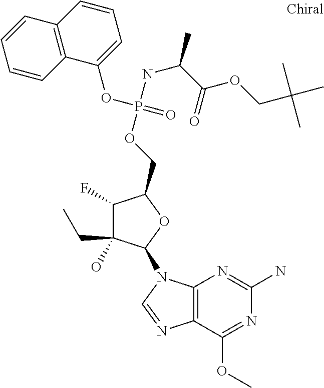 [SMILES: CC[C@@]1(O)[C@H](F)[C@@H](COP(=O)(N[C@@H](C)C(=O)OCC(C)(C)C)Oc2cccc3ccccc23)O[C@H]1n1cnc2c(OC)nc(N)nc21] |
| 4 | 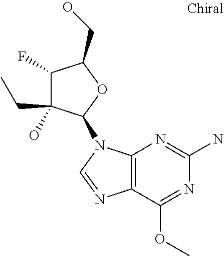 [SMILES: CC[C@@]1(O)[C@H](F)[C@@H](CO)O[C@H]1n1cnc2c(OC)nc(N)nc21] |
| 5 | 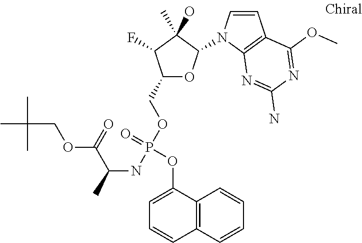 [SMILES: COc1nc(N)nc2c1ccn2[C@@H]1O[C@H](COP(=O)(N[C@@H](C)C(=O)OCC(C)(C)C)Oc2cccc3ccccc23)[C@H](F)[C@@]1(C)O] |
| 6 | 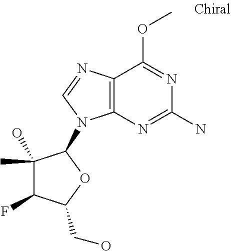 [SMILES: COc1nc(N)nc2c1ncn2[C@H]1O[C@H](CO)[C@@H](F)[C@]1(C)O] |
| 7 | 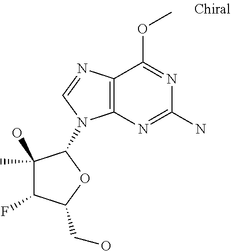 [SMILES: COc1nc(N)nc2c1ncn2[C@@H]1O[C@H](CO)[C@H](F)[C@@]1(C)O] |
| 8 | 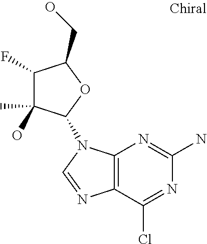 [SMILES: C[C@]1(O)[C@H](F)[C@@H](CO)O[C@@H]1n1cnc2c(Cl)nc(N)nc21] |

While the invention has been described with reference to particularly preferred embodiments and examples, those skilled in the art recognize that various modifications may be made to the invention without departing from the spirit and scope thereof.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

### ABBREVIATIONS AND ACRONYMS

A number of abbreviations and acronyms are used herein, and a full description of these are provided as follows:
ACN acetonitrile
AIBN azobisisobutyronitrile
anhy anhydrous
Bn benzyl (phenylmethyl)
Boc benzyloxycarbonyl
BSA benzenesulfonic acid
Bu butyl
n-BuOH n-butanol
t-BuOH tert-butanol
t-BuOK potassium-tert-butoxide
tert-BuMgCl tert-butylmagnesium chloride
CDCl₃ deuterochloroform
CD3OD methanol-d4
CI-MS chemical ionization mass spectrometry
13C NMR carbon-13 nuclear magnetic resonance spectroscopy
conc concentrated
d doublet (NMR)
dd doublet of doublets (NMR)
ddd double doublet of doublets (nmr
DBU diaza(1,3)bicyclo[5.4.0]undecane
DCC dicyclohexylcarbodiimide
DCM dichloromethane
DMAP 4-dimethylaminopyridine
DMF N,N-dimethylformamide
DMSO dimethyl sulfoxide
dt doublet of triplets (NMR)
EDCI 1-(3-dimethylaminopropyl)-3-ethylcarbodiimide hydrochloride
ee enantiomeric excess
El-MS electron impact mass spectrometry
equiv equivalent(s)
ESI electrospray ionization
ES-MS electrospray mass spectrometry
Et3N triethylamine
Et2O ethyl ether
EtOAc ethyl acetate
EtOH ethanol
g gram(s)
GC-MS gas chromatography-mass spectrometry
h hour
HCV hepatitis C virus
1H NMR proton nuclear magnetic resonance spectroscopy
HPLC high performance liquid chromatography
HRMS high resolution mass spectrometry
IMPDH inosine 5′monophosphate dehydroxygenase
J NMR coupling constant
LC/MS liquid chromatography-mass spectrometry
LG leaving group
LHMDS Lithium hex amethyldisilazide
m multiplet (NMR)
MDI methylenediphenyldisocyanate
Me methyl
MeOH methanol
mg milligram
MHz megahertz
mL milliliter
mmol millimole
mp melting point
MTBE methyl t-butyl ether
NaOMe sodium methoxide
NBS N-Bromo succinimide
NCS N-Chloro succinimide
NIS N-Iodo succinimide
NMI N-methylimidazole
NMO N-methylmorpholine-N-oxide
NMR nuclear magnetic resonance spectroscopy 31P NMR phosphorous-31 nuclear magnetic resonance spectroscopy
ppm parts per million
q quartet (NMR)
pTSA p-toluenesulfonic acid
RBV ribavirin
Red-Al® sodium bis(2-methoxyethoxy) aluminumhydride
Rf retention factor (TLC)
rt room temperature
singlet (NMR)
t triplet (NMR)
TBAF tetra-n-butylammonium fluoride
TBPPS tetra-n-butylphosphonium persulfate
TEA triethylamine
TFA trifluoroacetic acid
THF tetrahydrofuran
TMS tetramethylsilane
TMSCl trimethylsilyl chloride
TMSI trimethylsilyl iodide
TMSOTf trimethylsilyl trifluoromethanesulfonate
tR retention time
TLC thin layer chromatography
TOF ES+ time of flight electrospray positive ionization
UV ultraviolet
VCD Vibrational Circular Dichroism

What is claimed is:

1. A compound of formula (I) having the structure: 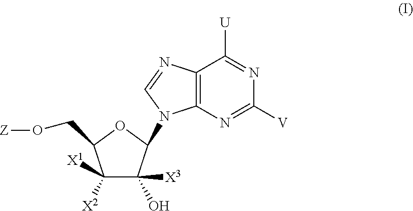
wherein
U and V are each independently selected from the group consisting of
hydrogen
OH
Cl
Br
I
OR1
NH2
NHR2
NR2R3
SH
and
SR4;
Wherein R1, R2, R3, and R4 are independently C1-C6 alkyl or aryl(C1-C3)alkyl; or
R2 and R3, together with the nitrogen atom to which they are attached, may join to form a 4-6 membered ring;
X1 is H or F;
X2 is F or H, with the requirement that X1≠X2;
X3 is CH3 or C1-C6 alkyl;
Z is selected from the group consisting of
hydrogen
—P(O)(OAr)NHR5
—P(O)(NHR5)2
—P(O)(NHR5)(NHR6)
—P(O)(OH)NHR6
—P(O)(OH)2 (monophosphate)
and
—P(O)(OH)—O—P(O)(OH)—O—P(O)(OH)2 (triphosphate);
wherein
R5 and R6 are independently
—C(R7)(R8)C(O)OR9;
wherein
R7 and R8 are independently
hydrogen,
alkyl,
aryl(C1-C6)alkyl,
or
phenyl;
R9 is independently C1-C6 alkyl,
aryl(C1-6)alkyl,
or
(4-pyranyl);
and
Ar is independently selected from the group consisting of
phenyl
1-naphthyl
2-naphthyl 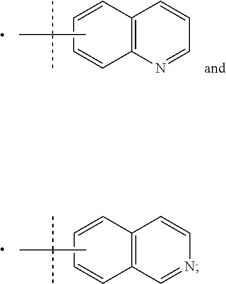
and tautormers and pharmaceutically acceptable salts thereof.

2. A pharmaceutical composition comprising the compound of claim 1 and a pharmaceutically acceptable carrier, excipient or diluent.

3. The compound of claim 1 wherein the compound has a specific formula selected from the group consisting of the following: 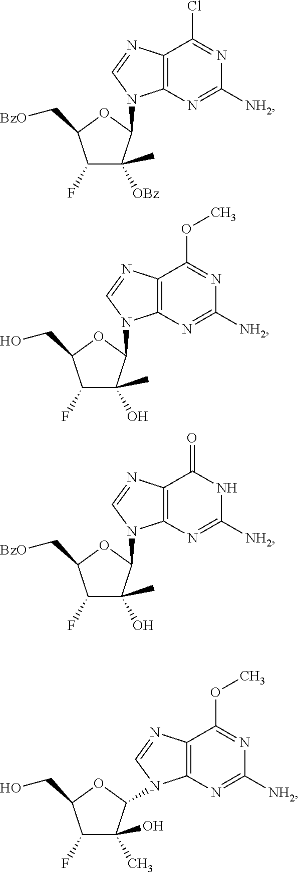 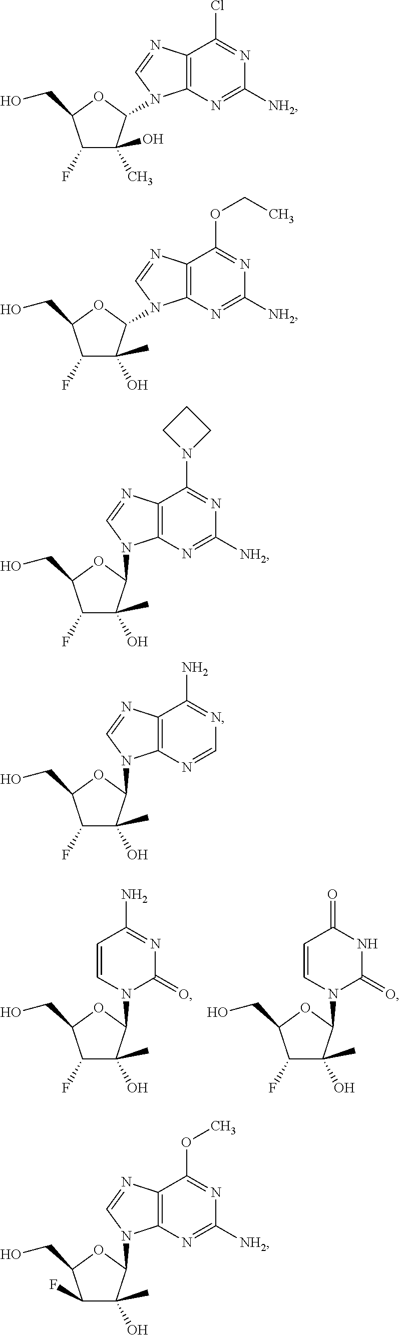 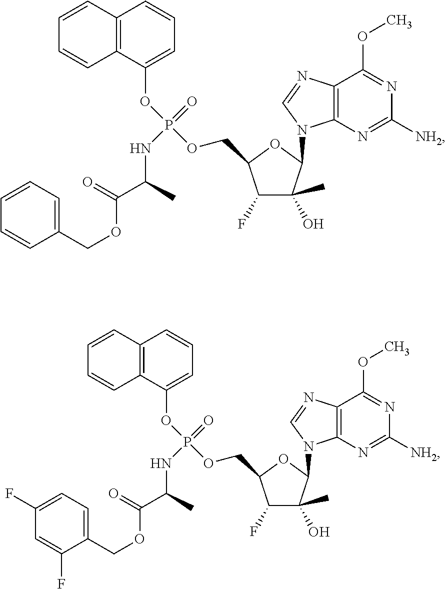 [SMILES: COc1nc(N)nc2c1ncn2[C@@H]1O[C@H](COP(=O)(N[C@@H](C)C(=O)OCc2ccccc2)Oc2cccc3ccccc23)[C@@H](F)[C@@]1(C)O ; COc1nc(N)nc2c1ncn2[C@@H]1O[C@H](COP(=O)(N[C@@H](C)C(=O)OCc2ccc(F)cc2F)Oc2cccc3ccccc23)[C@@H](F)[C@@]1(C)O] 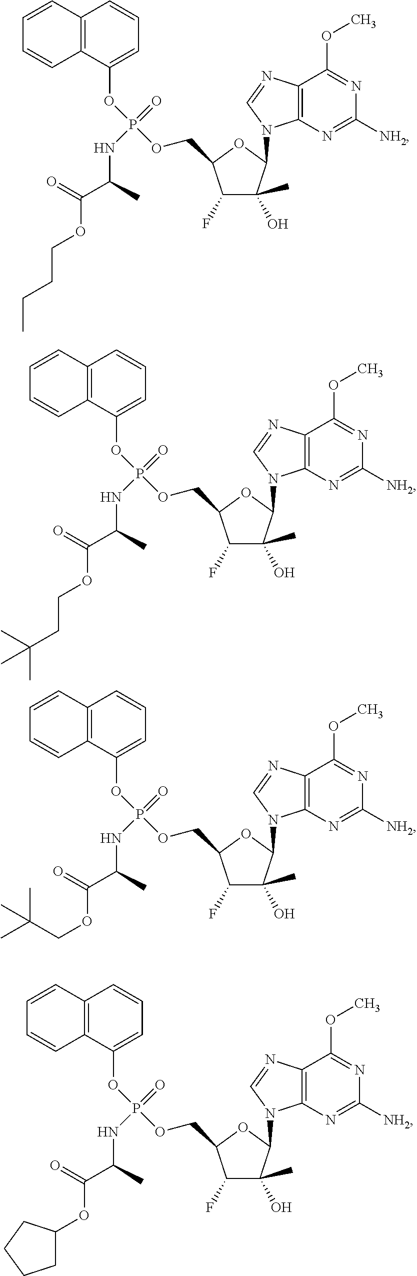 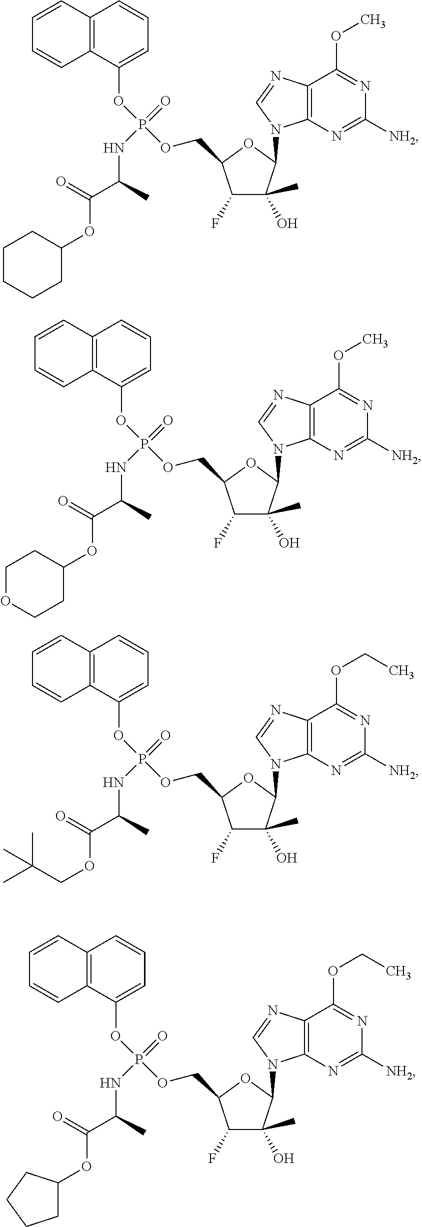 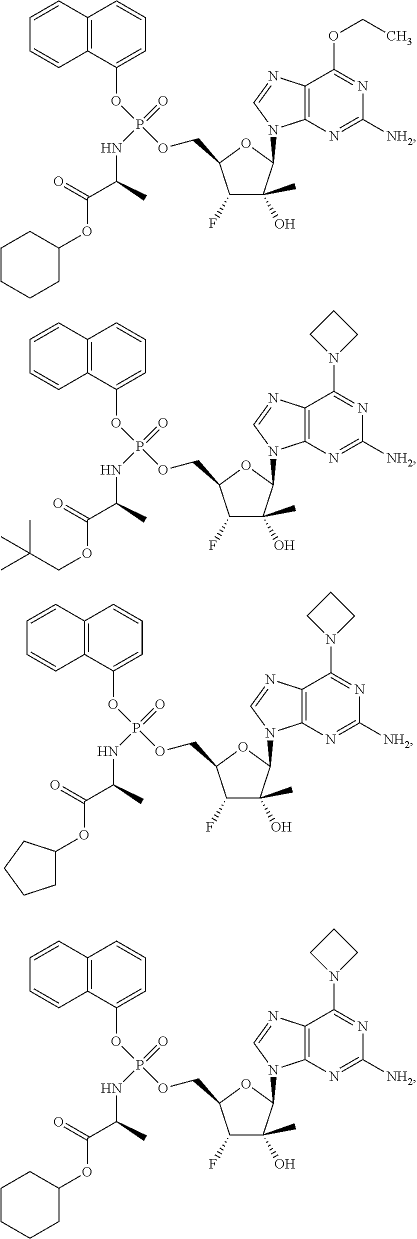 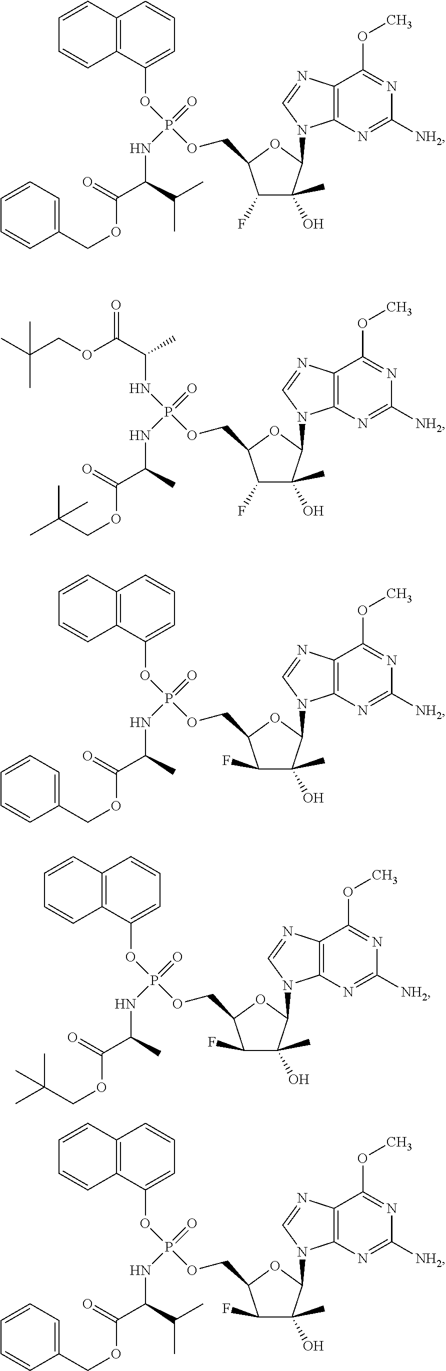 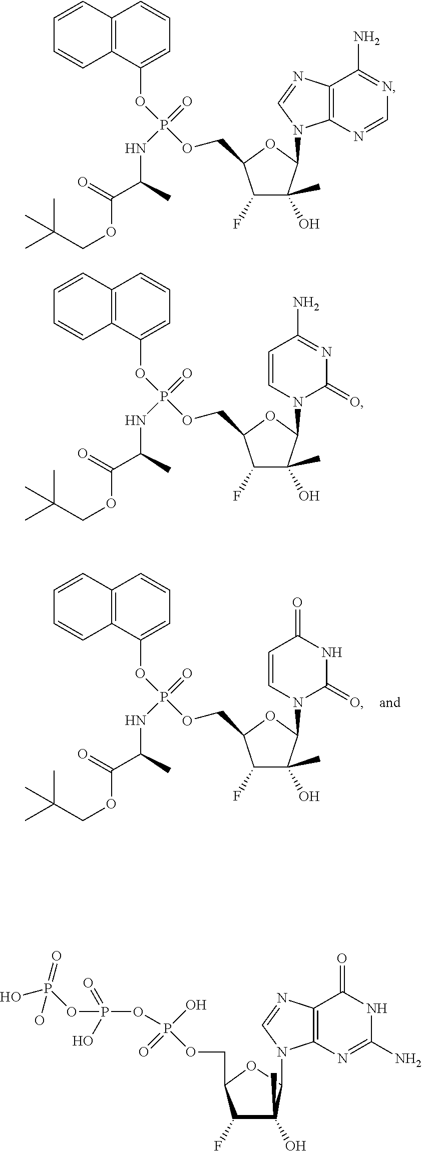
and tautomers and pharmaceutically acceptable salts thereof.

4. A pharmaceutical composition comprising the compound of claim 3 and a pharmaceutically acceptable carrier, excipient or diluent.

5. The compound according to claim 1 wherein the compound is selected from the group consisting of the following:
((2R,3R,4S,5R)-5-(2-Amino-6-chloro-9H-purin-9-yl)-4-(benzoyloxy)-3-fluoro-4-methyltetrahydrofuran-2-yl)methyl benzoate,
(2R,3S,4R,5R)-2-(2-Amino-6-methoxy-9H-purin-9-yl)-4-fluoro-5-(hydroxymethyl)-3-methyltetrahydrofuran-3-ol,
2-Amino-9-((2R,3S,4R,5R)-4-fluoro-3-hydroxy-5-(hydroxymethyl)-3-methyltetrahydrofuran-2-yl)-1H-purin-6(9H)-one,
(2S,3R,4R,5R)-2-(2-amino-6-methoxy-9H-purin-9-yl)-4-fluoro-5-(hydroxymethyl)-3-methyltetrahydrofuran-3-ol,
(2S,3R,4R,5R)-2-(2-amino-6-chloro-9H-purin-9-yl)-4-fluoro-5-(hydroxymethyl)-3-methyltetrahydrofuran-3-ol,
(2R,3S,4R,5R)-2-(2-Amino-6-ethoxy-9H-purin-9-yl)-4-fluoro-5-(hydroxymethyl)-3-methyltetrahydrofuran-3-ol,
(3S,4R,5R)-2-(2-Amino-6-(azetidin-1-yl)-9H-purin-9-yl)-4-fluoro-5-(hydroxymethyl)-3-methyltetrahydrofuran-3-ol,
(2R,3S,4R,5R)-2-(6-Amino-9H-purin-9-yl)-4-fluoro-5-(hydroxymethyl)-3-methyltetrahydrofuran-3-ol,
4-Amino-1-((2R,3S,4R,5R)-4-fluoro-3-hydroxy-5-(hydroxymethyl)-3-methyltetrahydrofuran-2-yl)pyrimidin-2(1H)-one,
1-((2R,3S,4R,5R)-4-Fluoro-3-hydroxy-5-(hydroxymethyl)-3-methyltetrahydrofuran-2-yl)pyrimidine-2,4(1H,3H)-dione,
(2R,3S,4S,5R)-2-(2-Amino-6-methoxy-9H-purin-9-yl)-4-fluoro-5-(hydroxymethyl)-3-methyltetrahydrofuran-3-ol,
(2S)-Benzyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-2,4-Difluorobenzyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Butyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-3,3-Dimethylbutyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Neopentyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Cyclopentyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Cyclohexyl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Tetrahydro-2H-pyran-4-yl 2-((((2R,3R,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Neopentyl 2-((((2R,3R,4S)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate,
(2S)-Cyclopentyl 2-((((2R,3R,4S)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate,
(2S)-Cyclohexyl 2-((((2R,3R,4S)-5-(2-amino-6-ethoxy-9H-purin-9-yl)-3-fluoro-4-hydroxy-4-methyltetrahydrofuran-2-yl)methoxy)(naphthalen-1-yloxy)phosphorylamino)propanoate,
(2S)—Neopentyl 2-((((2R,3R,4S)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate,
(2S)-Cyclopentyl 2-((((2R,3R,4S)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate,
(2S)-Cyclohexyl 2-((((2R,3R,4S)-5-(2-amino-6-(azetidin-1-yl)-9H-purin-9-yl)-3-fluoro-4-hydroxy-4-methyltetrahydrofuran-2-yl)methoxy)(naphthalen-1-yloxy)phosphorylamino)propanoate,
(2S)-Benzyl 2-((((2R,3R,4S,5R)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate,
(2S,2′S)—Neopentyl 2,2′-((((2R,3R,4S,5R)-5-(2-amino-6-methoxy-9H-purin-9-yl)-3-fluoro-4-hydroxy-4-methyltetrahydrofuran-2-yl)methoxy)phosphoryl)bis(azanediyl)dipropanoate,
(2S)-Benzyl 2-((((2R,3S,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Neopentyl 2-((((2R,3S,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)propanoate,
(2S)-Benzyl 2-((((2R,3S,4S)-5-(2-amino-6-methoxy-9H-purin-9-yl)-3-fluoro-4-hydroxy-4-methyltetrahydrofuran-2-yl)methoxy)(naphthalen-1-yloxy)phosphorylamino)-3-methylbutanoate,
(2S)-Neopentyl 2-((((2R,3R,4S,5R)-5-(6-amino-9H-purin-9-yl)-3-fluoro-4-hydroxy-4-methyltetrahydrofuran-2-yl)methoxy)(naphthalen-1-yloxy)phosphorylamino)propanoate,
(2S)-Neopentyl 2-((((2R,3R,4S,5R)-5-(4-amino-2-oxopyrimidin-1(2H)-yl)-3-fluoro-4-hydroxy-4-methyltetrahydrofuran-2-yl)methoxy)(naphthalen-1-yloxy)phosphorylamino)propanoate,
(2S)-Neopentyl 2-((((2R,3R,4S,5R)-5-(2,4-dioxo-3,4-dihydropyrimidin-1(2H)-yl)-3-fluoro-4-hydroxy-4-methyltetrahydrofuran-2-yl)methoxy)(naphthalen-1-yloxy)phosphorylamino)propanoate, and
((2R,5R)-5-(2-Amino-6-oxo-1H-purin-9(6H)-yl)-3-fluoro-4-hydroxy-4-methyltetrahydrofuran-2-yl)methyl tetrahydrogen triphosphate.

6. A pharmaceutical composition comprising the compound of claim 1 and a therapeutically effective amount of an agent active against hepatitis C virus.

7. The composition of claim 6 wherein said agent active against hepatitis C virus is interferon-alpha or pegylated interferon-alpha alone or in combination with ribavirin or levovirin.

8. The composition of claim 7 wherein interferon-alpha is selected from the group consisting of recombinant interferon-α2a, interferon-α2b, a consensus interferon, and a purified interferon-α product.

9. The composition of claim 6 wherein said agent active against hepatitis C virus is selected from the group consisting of ribavirin, levovirin, viramidine, thymosin alpha-1, an inhibitor of HCV NS3 serine protease, interferon-α, pegylated interferon-α (peginterferon-α), and combinations thereof.

10. The composition of claim 6 wherein said agent active against hepatitis C virus is an agent that inhibits a material selected from the group consisting of HCV proteases, HCV polymerase, HCV helicase, HCV NS4B protein, HCV entry, HCV assembly, HCV egress, HCV NS5A protein, and inosine 5′-monophosphate dehydrogenase.

11. The composition of claim 6 wherein said agent active against hepatitis C virus is a nucleoside analog for the treatment of an HCV infection.

12. The composition of claim 6 wherein said agent active against hepatitis C virus is selected from the group consisting of Omega IFN, BILN-2061, Roferon A, Pegasys, Pegasys/Ribaravin, CellCept, Wellferon, Albuferon-α, Levovirin, IDN-6556, IP-501, Actimmune, Infergen A, ISIS 14803, JTK-003, Pegasys/Ceplene, Ceplene, Civacir, Intron A/Zadaxin, Levovirin, Viramidine, Heptazyme, Intron A, PEG-Intron, Rebetron, Ribavirin, PEG-Intron/Ribavirin, Zadazim, Rebif, IFN-β/EMZ701, T67, VX-497, VX-950/LY-5703 10, Omniferon, XTL-002, SCH 503034, isatoribine and its prodrugs ANA971 and ANA975, R1479, Valopicitabine, NIM811, and Actilon.

13. A compound of formula (II) or (III) having the structure: 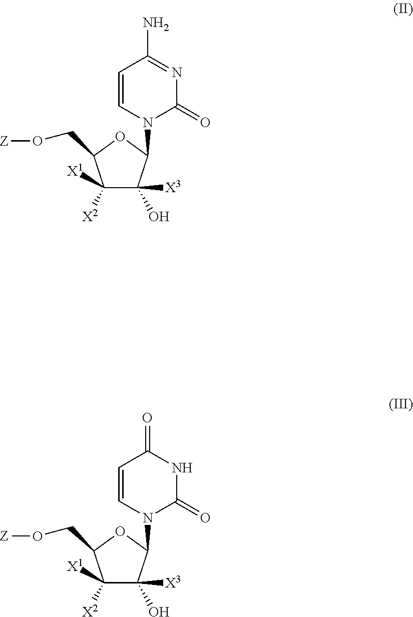
wherein
X1 is H or F;
X2 is F or H, with the requirement that X1≠X2;
X3 is CH3 or C1-C6 alkyl;
Z is selected from the group consisting of
hydrogen
—P(O)(OAr)NHR5
—P(O)(NHR5)2
—P(O)(NHR5)(NHR6)
—P(O)(OH)NHR6
—P(O)(OH)2 (monophosphate)
and
—P(O)(OH)—O—P(O)(OH)—O—P(O)(OH)2 (triphosphate)
wherein
R5 and R6 are independently
—C(R7)(R8)C(O)OR9;
wherein
R7 and R8 are independently
hydrogen,
Alkyl,
aryl(C1-C6)alkyl,
or
phenyl;
R9 is independently C1-C6 alkyl or aryl(C1-C6)alkyl;
and
Ar is independently selected from the group consisting of
phenyl
1-naphthyl
2-naphthyl 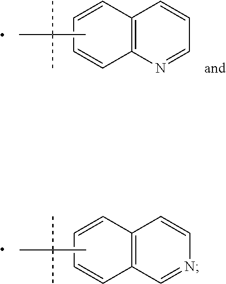
and the tautomers and the pharmaceutically acceptable salts thereof.

14. A pharmaceutical composition comprising the compound of claim 13 and a pharmaceutically acceptable carrier, excipient or diluent.

15. A method for treating a viral infection in a mammal mediated at least in part by a virus in the Flaviviridae family of viruses comprising administering to a mammal in need thereof an effective amount of the compound of claim 1.

16. The method of claim 15, wherein said virus is hepatitis C virus.

17. A method for treating a viral infection in a mammal mediated at least in part by a virus in the Flaviviridae family of viruses comprising administering to a mammal in need thereof an effective amount of the pharmaceutical composition of claim 2.

18. The method of claim 17, wherein said virus is hepatitis C virus.

19. A method for treating a viral infection in a mammal mediated at least in part by a virus in the Flaviviridae family of viruses comprising administering to a mammal in need thereof an effective amount of the compound of claim 13.

20. The method of claim 19, wherein said virus is hepatitis C virus.

21. A method for treating a viral infection in a mammal mediated at least in part by a virus in the Flaviviridae family of viruses comprising administering to a mammal in need thereof an effective amount of the pharmaceutical composition of claim 14.

22. The method of claim 21, wherein said virus is hepatitis C virus.

23. A method for treating a hepatitis C viral infection in a mammal comprising administering to a mammal in need thereof an effective amount of the compound of claim 1.

24. The method of claim 23 wherein the compound is administered in combination with a therapeutically effective amount of one or more agents active against hepatitis C virus.

25. The method of claim 24 wherein said agent active against hepatitis C virus is interferon-alpha or pegylated interferon-alpha alone or in combination with ribavirin or levovirin.

26. The method of claim 25 wherein interferon-alpha is selected from the group consisting of recombinant interferon-α2a, interferon-α2b, a consensus interferon, and a purified interferon-α product.

27. The method of claim 24 wherein said agent active against hepatitis C virus is selected from the group consisting of ribavirin, levovirin, viramidine, thymosin alpha-1, an inhibitor of HCV NS3 serine protease, interferon-α, pegylated interferon-α (peginterferon-α), and combinations thereof.

28. The method of claim 24 wherein said agent active against hepatitis C virus is an agent that inhibits a material selected from the group consisting of HCV proteases, HCV polymerase, HCV helicase, HCV NS4B protein, HCV entry, HCV assembly, HCV egress, HCV NS5A protein, and inosine 5′-monophosphate dehydrogenase.

29. The method of claim 24 wherein said agent active against hepatitis C virus is a nucleoside analog for the treatment of an HCV infection.

30. The method of claim 24 wherein said agent active against hepatitis C virus is selected from the group consisting of Omega IFN, BILN-2061, Roferon A, Pegasys, Pegasys/Ribaravin, CellCept, Wellferon, Albuferon-α, Levovirin, IDN-6556, IP-501, Actimmune, Infergen A, ISIS 14803, JTK-003, Pegasys/Ceplene, Ceplene, Civacir, Intron A/Zadaxin, Levovirin, Viramidine, Heptazyme, Intron A, PEG-Intron, Rebetron, Ribavirin, PEG-Intron/Ribavirin, Zadazim, Rebif, IFN-β/EMZ701, T67, VX-497, VX-950/LY-5703 10, Omniferon, XTL-002, SCH 503034, isatoribine and its prodrugs ANA971 and ANA975, R1479, Valopicitabine, NIM811, and Actilon.

31. The method of claim 23 wherein the compound is administered in combination with a therapeutically effective amount of one or more agents active against RNA-dependent RNA virus.

32. A method for treating a hepatitis C viral infection in a mammal comprising administering to a mammal in need thereof an effective amount of the pharmaceutical composition of claim 2.

33. The method of claim 32 wherein the composition is administered in combination with a therapeutically effective amount of one or more agents active against hepatitis C virus.

34. The compound according to claim 1 wherein the compound includes different diastereomers around phosphorous in formula I.

35. The compound according to claim 34 wherein the compound includes a mixture of two phosphorous diastereomers in any proportion from 1:99 to 99:1.

36. The compound according to claim 13 wherein the compound includes different diastereomers around phosphorous in formula II or III.

37. The compound according to claim 36 wherein the compound includes a mixture of two phosphorous diastereomers in any proportion from 1:99 to 99:1.
